SEQUENCE LISTING (1276 sequences)

SEQ ID NO: 1 - protein, 9 aa, Human immunodeficiency virus
SLKPCVKLT

SEQ ID NO: 2 - protein, 9 aa, Human immunodeficiency virus
LKPCVKLTP

SEQ ID NO: 3 - protein, 9 aa, Human immunodeficiency virus
KPCVKLTPL

SEQ ID NO: 4 - protein, 9 aa, Human immunodeficiency virus
PCVKLTPLC

SEQ ID NO: 5 - protein, 9 aa, Human immunodeficiency virus
CVKLTPLCV

SEQ ID NO: 6 - protein, 9 aa, Human immunodeficiency virus
VKLTPLCVS

SEQ ID NO: 7 - protein, 9 aa, Human immunodeficiency virus
KLTPLCVSL

SEQ ID NO: 8 - protein, 9 aa, Human immunodeficiency virus
LTPLCVSLK

SEQ ID NO: 9 - protein, 9 aa, Human immunodeficiency virus
TPLCVSLKC

SEQ ID NO: 10 - protein, 9 aa, Human immunodeficiency virus
PLCVSLKCT

SEQ ID NO: 11 - protein, 9 aa, Human immunodeficiency virus
LCVSLKCTD

SEQ ID NO: 12 - protein, 9 aa, Human immunodeficiency virus
CVSLKCTDL

SEQ ID NO: 13 - protein, 9 aa, Human immunodeficiency virus
VSLKCTDLK

SEQ ID NO: 14 - protein, 9 aa, Human immunodeficiency virus
SLKCTDLKN

SEQ ID NO: 15 - protein, 9 aa, Human immunodeficiency virus
LKCTDLKND

SEQ ID NO: 16 - protein, 9 aa, Human immunodeficiency virus
KCTDLKNDT

SEQ ID NO: 17 - protein, 9 aa, Human immunodeficiency virus
CTDLKNDTN

SEQ ID NO: 18 - protein, 9 aa, Human immunodeficiency virus
TDLKNDTNT

SEQ ID NO: 19 - protein, 9 aa, Human immunodeficiency virus
DLKNDTNTN

SEQ ID NO: 20 - protein, 9 aa, Human immunodeficiency virus
LKNDTNTNS

SEQ ID NO: 21 - protein, 9 aa, Human immunodeficiency virus
KNDTNTNSS

SEQ ID NO: 22 - protein, 9 aa, Human immunodeficiency virus
NDTNTNSSS

SEQ ID NO: 23 - protein, 9 aa, Human immunodeficiency virus
DTNTNSSSG

SEQ ID NO: 24 - protein, 9 aa, Human immunodeficiency virus
TNTNSSSGR

SEQ ID NO: 25 - protein, 9 aa, Human immunodeficiency virus
NTNSSSGRM

SEQ ID NO: 26 - protein, 9 aa, Human immunodeficiency virus
TNSSSGRMI

SEQ ID NO: 27 - protein, 9 aa, Human immunodeficiency virus
NSSSGRMIM

SEQ ID NO: 28 - protein, 9 aa, Human immunodeficiency virus
SLKPCVKLT

SEQ ID NO: 29 - protein, 9 aa, Human immunodeficiency virus
LKPCVKLTP

SEQ ID NO: 30 - protein, 9 aa, Human immunodeficiency virus
KPCVKLTPL

SEQ ID NO: 31 - protein, 9 aa, Human immunodeficiency virus
PCVKLTPLC

SEQ ID NO: 32 - protein, 9 aa, Human immunodeficiency virus
CVKLTPLCV

SEQ ID NO: 33 - protein, 9 aa, Human immunodeficiency virus
VKLTPLCVT

SEQ ID NO: 34 - protein, 9 aa, Human immunodeficiency virus
KLTPLCVTL

SEQ ID NO: 35 - protein, 9 aa, Human immunodeficiency virus
LTPLCVTLN

SEQ ID NO: 36 - protein, 9 aa, Human immunodeficiency virus
TPLCVTLNC

SEQ ID NO: 37 - protein, 9 aa, Human immunodeficiency virus
PLCVTLNCT

SEQ ID NO: 38 - protein, 9 aa, Human immunodeficiency virus
LCVTLNCTD

SEQ ID NO: 39 - protein, 9 aa, Human immunodeficiency virus
CVTLNCTDN

SEQ ID NO: 40 - protein, 9 aa, Human immunodeficiency virus
VTLNCTDNW

SEQ ID NO: 41 - protein, 9 aa, Human immunodeficiency virus
TLNCTDNWN

SEQ ID NO: 42 - protein, 9 aa, Human immunodeficiency virus
LNCTDNWNN

SEQ ID NO: 43 - protein, 9 aa, Human immunodeficiency virus
NCTDNWNNT

SEQ ID NO: 44 - protein, 9 aa, Human immunodeficiency virus
CTDNWNNTG

SEQ ID NO: 45 - protein, 9 aa, Human immunodeficiency virus
TDNWNNTGN

SEQ ID NO: 46 - protein, 9 aa, Human immunodeficiency virus
DNWNNTGNV

SEQ ID NO: 47 - protein, 9 aa, Human immunodeficiency virus
NWNNTGNVS

SEQ ID NO: 48 - protein, 9 aa, Human immunodeficiency virus
WNNTGNVSD

SEQ ID NO: 49 - protein, 9 aa, Human immunodeficiency virus
NNTGNVSDS

SEQ ID NO: 50 - protein, 9 aa, Human immunodeficiency virus
NTGNVSDSS

SEQ ID NO: 51 - protein, 9 aa, Human immunodeficiency virus
TGNVSDSSW

SEQ ID NO: 52 - protein, 9 aa, Human immunodeficiency virus
GNVSDSSWK

SEQ ID NO: 53 - protein, 9 aa, Human immunodeficiency virus
TSVNSNSSG

SEQ ID NO: 54 - protein, 9 aa, Human immunodeficiency virus
SVNSNSSGG

SEQ ID NO: 55 - protein, 11 aa, Human immunodeficiency virus
MGARASVLSGG

SEQ ID NO: 56 - protein, 10 aa, Human immunodeficiency virus
WEKIRLRPGG

SEQ ID NO: 57 - protein, 11 aa, Human immunodeficiency virus
VWASRELERFA

SEQ ID NO: 58 - protein, 9 aa, Human immunodeficiency virus
SQNYPIVQN

SEQ ID NO: 59 - protein, 11 aa, Human immunodeficiency virus
SPRTLNAWVKV

SEQ ID NO: 60 - protein, 13 aa, Human immunodeficiency virus
EEKAFSPEVIPMF

SEQ ID NO: 61 - protein, 29 aa, Human immunodeficiency virus
ALSEGATPQDLNTMLNTVGGHQAAMQMLK

SEQ ID NO: 62 - protein, 11 aa, Human immunodeficiency virus
TINEEAAEWDR

SEQ ID NO: 63 - protein, 23 aa, Human immunodeficiency virus
PGQMREPRGSDIAGTTSTLQEQI

SEQ ID NO: 64 - protein, 11 aa, Human immunodeficiency virus
NPPIPVGEIYK

SEQ ID NO: 65 - protein, 11 aa, Human immunodeficiency virus
GLNKIVRMYSP

SEQ ID NO: 66 - protein, 23 aa, Human immunodeficiency virus
QGPKEPFRDYVDRFYKTLRAEQA

SEQ ID NO: 67 - protein, 27 aa, Human immunodeficiency virus
VKNWMTETLLVQNANPDCKTILKALGP

SEQ ID NO: 68 - protein, 16 aa, Human immunodeficiency virus
ATLEEMMTACQGVGGP

SEQ ID NO: 69 - protein, 11 aa, Human immunodeficiency virus
HKARVLAEAMS

SEQ ID NO: 70 - protein, 10 aa, Human immunodeficiency virus
KCFNCGKEGH

SEQ ID NO: 71 - protein, 9 aa, Human immunodeficiency virus
NFLGKIWPS

SEQ ID NO: 72 - protein, 9 aa, Human immunodeficiency virus
KGRPGNFLQ

SEQ ID NO: 73 - protein, 9 aa, Human immunodeficiency virus
PQITLWQRP

SEQ ID NO: 74 - protein, 15 aa, Human immunodeficiency virus
KEALLDTGADDTVLE

SEQ ID NO: 75 - protein, 10 aa, Human immunodeficiency virus
PKMIGGIGGF

SEQ ID NO: 76 - protein, 10 aa, Human immunodeficiency virus
IGGIGGFIKV

SEQ ID NO: 77 - protein, 25 aa, Human immunodeficiency virus
GCTLNFPISPIETVPVKLKPGMDGP

SEQ ID NO: 78 - protein, 14 aa, Human immunodeficiency virus
VKQWPLTEEKIKAL

SEQ ID NO: 79 - protein, 15 aa, Human immunodeficiency virus
GKISKIGPENPYNTP

SEQ ID NO: 80 - protein, 12 aa, Human immunodeficiency virus
WRKLVDFRELNK

SEQ ID NO: 81 - protein, 19 aa, Human immunodeficiency virus
TQDFWEVQLGIPHPAGLKK

SEQ ID NO: 82 - protein, 14 aa, Human immunodeficiency virus
KSVTVLDVGDAYFS

SEQ ID NO: 83 - protein, 11 aa, Human immunodeficiency virus
FRKYTAFTIPS

SEQ ID NO: 84 - protein, 26 aa, Human immunodeficiency virus
NNETPGIRYQYNVLPQGWKGSPAIFQ

SEQ ID NO: 85 - protein, 10 aa, Human immunodeficiency virus
SMTKILEPFR

SEQ ID NO: 86 - protein, 11 aa, Human immunodeficiency virus
DDLYVGSDLEI

SEQ ID NO: 87 - protein, 25 aa, Human immunodeficiency virus
KHQKEPPFLWMGYELHPDKWTVQPI

SEQ ID NO: 88 - protein, 26 aa, Human immunodeficiency virus
LPEKDSWTVNDIQKLVGKLNWASQIY

SEQ ID NO: 89 - protein, 19 aa, Human immunodeficiency virus
EAELELAENREILKEPVHG

SEQ ID NO: 90 - protein, 9 aa, Human immunodeficiency virus
FLDGIDKAQ

SEQ ID NO: 91 - protein, 9 aa, Human immunodeficiency virus
EIVASCDKC

SEQ ID NO: 92 - protein, 10 aa, Human immunodeficiency virus
CDKCQLKGEA

SEQ ID NO: 93 - protein, 21 aa, Human immunodeficiency virus
HGQVDCSPGIWQLDCTHLEGK

SEQ ID NO: 94 - protein, 28 aa, Human immunodeficiency virus
ILVAVHVASGYIEAEVIPAETGQETAYF

SEQ ID NO: 95 - protein, 10 aa, Human immunodeficiency virus
LKLAGRWPVK

SEQ ID NO: 96 - protein, 10 aa, Human immunodeficiency virus
VKAACWWAGI

SEQ ID NO: 97 - protein, 27 aa, Human immunodeficiency virus
ACWWAGIKQEFGIPYNPQSQGVVESMN

SEQ ID NO: 98 - protein, 10 aa, Human immunodeficiency virus
ELKKIIGQVR

SEQ ID NO: 99 - protein, 40 aa, Human immunodeficiency virus
IIGQVRDQAEHLKTAVQMAVFIHNFKRKGGIGGYSAGERI

SEQ ID NO: 100 - protein, 11 aa, Human immunodeficiency virus
KIQNFRVYYRD

SEQ ID NO: 101 - protein, 9 aa, Human immunodeficiency virus
FRVYYRDSR

SEQ ID NO: 102 - protein, 23 aa, Human immunodeficiency virus
PLWKGPAKLLWKGEGAVVIQDNS

SEQ ID NO: 103 - protein, 17 aa, Human immunodeficiency virus
KIIRDYGKQMAGDDCVA

SEQ ID NO: 104 - protein, 18 aa, Human immunodeficiency virus
MENRWQVMIVWQVDRMRI

SEQ ID NO: 105 - protein, 9 aa, Human immunodeficiency virus
SSEVHIPLG

SEQ ID NO: 106 - protein, 10 aa, Human immunodeficiency virus
TYWGLHTGER

SEQ ID NO: 107 - protein, 12 aa, Human immunodeficiency virus
WHLGQGVSIEWR

SEQ ID NO: 108 - protein, 13 aa, Human immunodeficiency virus
GHNKVGSLQYLAL

SEQ ID NO: 109 - protein, 11 aa, Human immunodeficiency virus
KLTEDRWNKPQ

SEQ ID NO: 110 - protein, 14 aa, Human immunodeficiency virus
MEQAPEDQGPQREP

SEQ ID NO: 111 - protein, 10 aa, Human immunodeficiency virus
WTLELLEELK

SEQ ID NO: 112 - protein, 11 aa, Human immunodeficiency virus
LEPWKHPGSQP

SEQ ID NO: 113 - protein, 15 aa, Human immunodeficiency virus
LGISYGRKKRRQRRR

SEQ ID NO: 114 - protein, 15 aa, Human immunodeficiency virus
EGTRQARRNRRRRWR

SEQ ID NO: 115 - protein, 10 aa, Human immunodeficiency virus
ERAEDSGNES

SEQ ID NO: 116 - protein, 26 aa, Human immunodeficiency virus
LWVTVYYGVPVWKEATTTLFCASDAK

SEQ ID NO: 117 - protein, 12 aa, Human immunodeficiency virus
HNVWATHACVPT

SEQ ID NO: 118 - protein, 15 aa, Human immunodeficiency virus
SLKPCVKLTPLCVTL

SEQ ID NO: 119 - protein, 11 aa, Human immunodeficiency virus
NVSTVQCTHGI

SEQ ID NO: 120 - protein, 15 aa, Human immunodeficiency virus
PVVSTQLLLNGSLAE

SEQ ID NO: 121 - protein, 10 aa, Human immunodeficiency virus
VGKAMYAPPI

SEQ ID NO: 122 - protein, 13 aa, Human immunodeficiency virus
DNWRSELYKYKVV

SEQ ID NO: 123 - protein, 9 aa, Human immunodeficiency virus
AKRRVVQRE

SEQ ID NO: 124 - protein, 16 aa, Human immunodeficiency virus
FLGFLGAAGSTMGAAS

SEQ ID NO: 125 - protein, 9 aa, Human immunodeficiency virus
LLSGIVQQQ

SEQ ID NO: 126 - protein, 17 aa, Human immunodeficiency virus
LQLTVWGIKQLQARVLA

SEQ ID NO: 127 - protein, 18 aa, Human immunodeficiency virus
DQQLLGIWGCSGKLICTT

SEQ ID NO: 128 - protein, 9 aa, Human immunodeficiency virus
WLWYIKIFI

SEQ ID NO: 129 - protein, 10 aa, Human immunodeficiency virus
AIAVAEGTDR

SEQ ID NO: 130 - protein, 9 aa, Human immunodeficiency virus
PQVPLRPMT

SEQ ID NO: 131 - protein, 12 aa, Human immunodeficiency virus
FPDWQNYTPGPG

SEQ ID NO: 132 - protein, 9 aa, Human immunodeficiency virus
FGWCFKLVP

SEQ ID NO: 133 - protein, 9 aa, Human immunodeficiency virus
MGARASVLS

SEQ ID NO: 134 - protein, 9 aa, Human immunodeficiency virus
GARASVLSG

SEQ ID NO: 135 - protein, 9 aa, Human immunodeficiency virus
ARASVLSGG

SEQ ID NO: 136 - protein, 9 aa, Human immunodeficiency virus
WEKIRLRPG

SEQ ID NO: 137 - protein, 9 aa, Human immunodeficiency virus
EKIRLRPGG

SEQ ID NO: 138 - protein, 9 aa, Human immunodeficiency virus
VWASRELER

SEQ ID NO: 139 - protein, 9 aa, Human immunodeficiency virus
WASRELERF

SEQ ID NO: 140 - protein, 9 aa, Human immunodeficiency virus
ASRELERFA

SEQ ID NO: 141 - protein, 9 aa, Human immunodeficiency virus
SQNYPIVQN

SEQ ID NO: 142 - protein, 9 aa, Human immunodeficiency virus
SPRTLNAWV

SEQ ID NO: 143 - protein, 9 aa, Human immunodeficiency virus
PRTLNAWVK

SEQ ID NO: 144 - protein, 9 aa, Human immunodeficiency virus
RTLNAWVKV

SEQ ID NO: 145 - protein, 9 aa, Human immunodeficiency virus
EEKAFSPEV

SEQ ID NO: 146 - protein, 9 aa, Human immunodeficiency virus
EKAFSPEVI

SEQ ID NO: 147 - protein, 9 aa, Human immunodeficiency virus
KAFSPEVIP

SEQ ID NO: 148 - protein, 9 aa, Human immunodeficiency virus
AFSPEVIPM

SEQ ID NO: 149 - protein, 9 aa, Human immunodeficiency virus
FSPEVIPMF

SEQ ID NO: 150 - protein, 9 aa, Human immunodeficiency virus
ALSEGATPQ

SEQ ID NO: 151 - protein, 9 aa, Human immunodeficiency virus
LSEGATPQD

SEQ ID NO: 152 - protein, 9 aa, Human immunodeficiency virus
SEGATPQDL

SEQ ID NO: 153 - protein, 9 aa, Human immunodeficiency virus
EGATPQDLN

SEQ ID NO: 154 - protein, 9 aa, Human immunodeficiency virus
GATPQDLNT

SEQ ID NO: 155 - protein, 9 aa, Human immunodeficiency virus
ATPQDLNTM

SEQ ID NO: 156 - protein, 9 aa, Human immunodeficiency virus
TPQDLNTML

SEQ ID NO: 157 - protein, 9 aa, Human immunodeficiency virus
PQDLNTMLN

SEQ ID NO: 158 - protein, 9 aa, Human immunodeficiency virus
QDLNTMLNT

SEQ ID NO: 159 - protein, 9 aa, Human immunodeficiency virus
DLNTMLNTV

SEQ ID NO: 160 - protein, 9 aa, Human immunodeficiency virus
LNTMLNTVG

SEQ ID NO: 161 - protein, 9 aa, Human immunodeficiency virus
NTMLNTVGG

SEQ ID NO: 162 - protein, 9 aa, Human immunodeficiency virus
TMLNTVGGH

SEQ ID NO: 163 - protein, 9 aa, Human immunodeficiency virus
MLNTVGGHQ

SEQ ID NO: 164 - protein, 9 aa, Human immunodeficiency virus
LNTVGGHQA

SEQ ID NO: 165 - protein, 9 aa, Human immunodeficiency virus
NTVGGHQAA

SEQ ID NO: 166 - protein, 9 aa, Human immunodeficiency virus
TVGGHQAAM

SEQ ID NO: 167 - protein, 9 aa, Human immunodeficiency virus
VGGHQAAMQ

SEQ ID NO: 168 - protein, 9 aa, Human immunodeficiency virus
GGHQAAMQM

SEQ ID NO: 169 - protein, 9 aa, Human immunodeficiency virus
GHQAAMQML

SEQ ID NO: 170 - protein, 9 aa, Human immunodeficiency virus
HQAAMQMLK

SEQ ID NO: 171 - protein, 9 aa, Human immunodeficiency virus
TINEEAAEW

SEQ ID NO: 172 - protein, 9 aa, Human immunodeficiency virus
INEEAAEWD

SEQ ID NO: 173 - protein, 9 aa, Human immunodeficiency virus
NEEAAEWDR

SEQ ID NO: 174 - protein, 9 aa, Human immunodeficiency virus
PGQMREPRG

SEQ ID NO: 175 - protein, 9 aa, Human immunodeficiency virus
GQMREPRGS

SEQ ID NO: 176 - protein, 9 aa, Human immunodeficiency virus
QMREPRGSD

SEQ ID NO: 177 - protein, 9 aa, Human immunodeficiency virus
MREPRGSDI

SEQ ID NO: 178 - protein, 9 aa, Human immunodeficiency virus
REPRGSDIA

SEQ ID NO: 179 - protein, 9 aa, Human immunodeficiency virus
EPRGSDIAG

SEQ ID NO: 180 - protein, 9 aa, Human immunodeficiency virus
PRGSDIAGT

SEQ ID NO: 181 - protein, 9 aa, Human immunodeficiency virus
RGSDIAGTT

SEQ ID NO: 182 - protein, 9 aa, Human immunodeficiency virus
GSDIAGTTS

SEQ ID NO: 183 - protein, 9 aa, Human immunodeficiency virus
SDIAGTTST

SEQ ID NO: 184 - protein, 9 aa, Human immunodeficiency virus
DIAGTTSTL

SEQ ID NO: 185 - protein, 9 aa, Human immunodeficiency virus
IAGTTSTLQ

SEQ ID NO: 186 - protein, 9 aa, Human immunodeficiency virus
AGTTSTLQE

SEQ ID NO: 187 - protein, 9 aa, Human immunodeficiency virus
GTTSTLQEQ

SEQ ID NO: 188 - protein, 9 aa, Human immunodeficiency virus
TTSTLQEQI

SEQ ID NO: 189 - protein, 9 aa, Human immunodeficiency virus
NPPIPVGEI

SEQ ID NO: 190 - protein, 9 aa, Human immunodeficiency virus
PPIPVGEIY

SEQ ID NO: 191 - protein, 9 aa, Human immunodeficiency virus
PIPVGEIYK

SEQ ID NO: 192 - protein, 9 aa, Human immunodeficiency virus
GLNKIVRMY

SEQ ID NO: 193 - protein, 9 aa, Human immunodeficiency virus
LNKIVRMYS

SEQ ID NO: 194 - protein, 9 aa, Human immunodeficiency virus
NKIVRMYSP

SEQ ID NO: 195 - protein, 9 aa, Human immunodeficiency virus
QGPKEPFRD

SEQ ID NO: 196 - protein, 9 aa, Human immunodeficiency virus
GPKEPFRDY

SEQ ID NO: 197 - protein, 9 aa, Human immunodeficiency virus
PKEPFRDYV

SEQ ID NO: 198 - protein, 9 aa, Human immunodeficiency virus
KEPFRDYVD

SEQ ID NO: 199 - protein, 9 aa, Human immunodeficiency virus
EPFRDYVDR

SEQ ID NO: 200 - protein, 9 aa, Human immunodeficiency virus
PFRDYVDRF

SEQ ID NO: 201 - protein, 9 aa, Human immunodeficiency virus
FRDYVDRFY

SEQ ID NO: 202 - protein, 9 aa, Human immunodeficiency virus
RDYVDRFYK

SEQ ID NO: 203 - protein, 9 aa, Human immunodeficiency virus
DYVDRFYKT

SEQ ID NO: 204 - protein, 9 aa, Human immunodeficiency virus
YVDRFYKTL

SEQ ID NO: 205 - protein, 9 aa, Human immunodeficiency virus
VDRFYKTLR

SEQ ID NO: 206 - protein, 9 aa, Human immunodeficiency virus
DRFYKTLRA

SEQ ID NO: 207 - protein, 9 aa, Human immunodeficiency virus
RFYKTLRAE

SEQ ID NO: 208 - protein, 9 aa, Human immunodeficiency virus
FYKTLRAEQ

SEQ ID NO: 209 - protein, 9 aa, Human immunodeficiency virus
YKTLRAEQA

SEQ ID NO: 210 - protein, 9 aa, Human immunodeficiency virus
VKNWMTETL

SEQ ID NO: 211 - protein, 9 aa, Human immunodeficiency virus
KNWMTETLL

SEQ ID NO: 212 - protein, 9 aa, Human immunodeficiency virus
NWMTETLLV

SEQ ID NO: 213 - protein, 9 aa, Human immunodeficiency virus
WMTETLLVQ

SEQ ID NO: 214 - protein, 9 aa, Human immunodeficiency virus
MTETLLVQN

SEQ ID NO: 215 - protein, 9 aa, Human immunodeficiency virus
TETLLVQNA

SEQ ID NO: 216 - protein, 9 aa, Human immunodeficiency virus
ETLLVQNAN

SEQ ID NO: 217 - protein, 9 aa, Human immunodeficiency virus
TLLVQNANP

SEQ ID NO: 218 - protein, 9 aa, Human immunodeficiency virus
LLVQNANPD

SEQ ID NO: 219 - protein, 9 aa, Human immunodeficiency virus
LVQNANPDC

SEQ ID NO: 220 - protein, 9 aa, Human immunodeficiency virus
VQNANPDCK

SEQ ID NO: 221 - protein, 9 aa, Human immunodeficiency virus
QNANPDCKT

SEQ ID NO: 222 - protein, 9 aa, Human immunodeficiency virus
NANPDCKTI

SEQ ID NO: 223 - protein, 9 aa, Human immunodeficiency virus
ANPDCKTIL

SEQ ID NO: 224 - protein, 9 aa, Human immunodeficiency virus
NPDCKTILK

SEQ ID NO: 225 - protein, 9 aa, Human immunodeficiency virus
PDCKTILKA

SEQ ID NO: 226 - protein, 9 aa, Human immunodeficiency virus
DCKTILKAL

SEQ ID NO: 227 - protein, 9 aa, Human immunodeficiency virus
CKTILKALG

SEQ ID NO: 228 - protein, 9 aa, Human immunodeficiency virus
KTILKALGP

SEQ ID NO: 229 - protein, 9 aa, Human immunodeficiency virus
ATLEEMMTA

SEQ ID NO: 230 - protein, 9 aa, Human immunodeficiency virus
TLEEMMTAC

SEQ ID NO: 231 - protein, 9 aa, Human immunodeficiency virus
LEEMMTACQ

SEQ ID NO: 232 - protein, 9 aa, Human immunodeficiency virus
EEMMTACQG

SEQ ID NO: 233 - protein, 9 aa, Human immunodeficiency virus
EMMTACQGV

SEQ ID NO: 234 - protein, 9 aa, Human immunodeficiency virus
MMTACQGVG

SEQ ID NO: 235 - protein, 9 aa, Human immunodeficiency virus
MTACQGVGG

SEQ ID NO: 236 - protein, 9 aa, Human immunodeficiency virus
TACQGVGGP

SEQ ID NO: 237 - protein, 9 aa, Human immunodeficiency virus
HKARVLAEA

SEQ ID NO: 238 - protein, 9 aa, Human immunodeficiency virus
KARVLAEAM

SEQ ID NO: 239 - protein, 9 aa, Human immunodeficiency virus
ARVLAEAMS

SEQ ID NO: 240 - protein, 9 aa, Human immunodeficiency virus
KCFNCGKEG

SEQ ID NO: 241 - protein, 9 aa, Human immunodeficiency virus
CFNCGKEGH

SEQ ID NO: 242 - protein, 9 aa, Human immunodeficiency virus
NFLGKIWPS

SEQ ID NO: 243 - protein, 9 aa, Human immunodeficiency virus
KGRPGNFLQ

SEQ ID NO: 244 - protein, 9 aa, Human immunodeficiency virus
PQVTLWQRP

SEQ ID NO: 245 - protein, 9 aa, Human immunodeficiency virus
KEALLDTGA

SEQ ID NO: 246 - protein, 9 aa, Human immunodeficiency virus
EALLDTGAD

SEQ ID NO: 247 - protein, 9 aa, Human immunodeficiency virus
ALLDTGADD

SEQ ID NO: 248 - protein, 9 aa, Human immunodeficiency virus
LLDTGADDT

SEQ ID NO: 249 - protein, 9 aa, Human immunodeficiency virus
LDTGADDTV

SEQ ID NO: 250 - protein, 9 aa, Human immunodeficiency virus
DTGADDTVL

SEQ ID NO: 251 - protein, 9 aa, Human immunodeficiency virus
TGADDTVLE

SEQ ID NO: 252 - protein, 9 aa, Human immunodeficiency virus
PKMIGGIGG

SEQ ID NO: 253 - protein, 9 aa, Human immunodeficiency virus
KMIGGIGGF

SEQ ID NO: 254 - protein, 9 aa, Human immunodeficiency virus
IGGIGGFIK

SEQ ID NO: 255 - protein, 9 aa, Human immunodeficiency virus
GGIGGFIKV

SEQ ID NO: 256 - protein, 9 aa, Human immunodeficiency virus
GCTLNFPIS

SEQ ID NO: 257 - protein, 9 aa, Human immunodeficiency virus
CTLNFPISP

SEQ ID NO: 258 - protein, 9 aa, Human immunodeficiency virus
TLNFPISPI

SEQ ID NO: 259 - protein, 9 aa, Human immunodeficiency virus
LNFPISPIE

SEQ ID NO: 260 - protein, 9 aa, Human immunodeficiency virus
NFPISPIET

SEQ ID NO: 261 - protein, 9 aa, Human immunodeficiency virus
FPISPIETV

SEQ ID NO: 262 - protein, 9 aa, Human immunodeficiency virus
PISPIETVP

SEQ ID NO: 263 - protein, 9 aa, Human immunodeficiency virus
ISPIETVPV

SEQ ID NO: 264 - protein, 9 aa, Human immunodeficiency virus
SPIETVPVK

SEQ ID NO: 265 - protein, 9 aa, Human immunodeficiency virus
PIETVPVKL

SEQ ID NO: 266 - protein, 9 aa, Human immunodeficiency virus
IETVPVKLK

SEQ ID NO: 267 - protein, 9 aa, Human immunodeficiency virus
ETVPVKLKP

SEQ ID NO: 268 - protein, 9 aa, Human immunodeficiency virus
TVPVKLKPG

SEQ ID NO: 269 - protein, 9 aa, Human immunodeficiency virus
VPVKLKPGM

SEQ ID NO: 270 - protein, 9 aa, Human immunodeficiency virus
PVKLKPGMD

SEQ ID NO: 271 - protein, 9 aa, Human immunodeficiency virus
VKLKPGMDG

SEQ ID NO: 272 - protein, 9 aa, Human immunodeficiency virus
KLKPGMDGP

SEQ ID NO: 273 - protein, 9 aa, Human immunodeficiency virus
VKQWPLTEE

SEQ ID NO: 274 - protein, 9 aa, Human immunodeficiency virus
KQWPLTEEK

SEQ ID NO: 275 - protein, 9 aa, Human immunodeficiency virus
QWPLTEEKI

SEQ ID NO: 276 - protein, 9 aa, Human immunodeficiency virus
WPLTEEKIK

SEQ ID NO: 277 - protein, 9 aa, Human immunodeficiency virus
PLTEEKIKA

SEQ ID NO: 278 - protein, 9 aa, Human immunodeficiency virus
LTEEKIKAL

SEQ ID NO: 279 - protein, 9 aa, Human immunodeficiency virus
GKISKIGPE

SEQ ID NO: 280 - protein, 9 aa, Human immunodeficiency virus
KISKIGPEN

SEQ ID NO: 281 - protein, 9 aa, Human immunodeficiency virus
ISKIGPENP

SEQ ID NO: 282 - protein, 9 aa, Human immunodeficiency virus
SKIGPENPY

SEQ ID NO: 283 - protein, 9 aa, Human immunodeficiency virus
KIGPENPYN

SEQ ID NO: 284 - protein, 9 aa, Human immunodeficiency virus
IGPENPYNT

SEQ ID NO: 285 - protein, 9 aa, Human immunodeficiency virus
GPENPYNTP

SEQ ID NO: 286 - protein, 9 aa, Human immunodeficiency virus
WRKLVDFRE

SEQ ID NO: 287 - protein, 9 aa, Human immunodeficiency virus
RKLVDFREL

SEQ ID NO: 288 - protein, 9 aa, Human immunodeficiency virus
KLVDFRELN

SEQ ID NO: 289 - protein, 9 aa, Human immunodeficiency virus
LVDFRELNK

SEQ ID NO: 290 - protein, 9 aa, Human immunodeficiency virus
TQDFWEVQL

SEQ ID NO: 291 - protein, 9 aa, Human immunodeficiency virus
QDFWEVQLG

SEQ ID NO: 292 - protein, 9 aa, Human immunodeficiency virus
DFWEVQLGI

SEQ ID NO: 293 - protein, 9 aa, Human immunodeficiency virus
FWEVQLGIP

SEQ ID NO: 294 - protein, 9 aa, Human immunodeficiency virus
WEVQLGIPH

SEQ ID NO: 295 - protein, 9 aa, Human immunodeficiency virus
EVQLGIPHP

SEQ ID NO: 296 - protein, 9 aa, Human immunodeficiency virus
VQLGIPHPA

SEQ ID NO: 297 - protein, 9 aa, Human immunodeficiency virus
QLGIPHPAG

SEQ ID NO: 298 - protein, 9 aa, Human immunodeficiency virus
LGIPHPAGL

SEQ ID NO: 299 - protein, 9 aa, Human immunodeficiency virus
GIPHPAGLK

SEQ ID NO: 300 - protein, 9 aa, Human immunodeficiency virus
IPHPAGLKK

SEQ ID NO: 301 - protein, 9 aa, Human immunodeficiency virus
KSVTVLDVG

SEQ ID NO: 302 - protein, 9 aa, Human immunodeficiency virus
SVTVLDVGD

SEQ ID NO: 303 - protein, 9 aa, Human immunodeficiency virus
VTVLDVGDA

SEQ ID NO: 304 - protein, 9 aa, Human immunodeficiency virus
TVLDVGDAY

SEQ ID NO: 305 - protein, 9 aa, Human immunodeficiency virus
VLDVGDAYF

SEQ ID NO: 306 - protein, 9 aa, Human immunodeficiency virus
LDVGDAYFS

SEQ ID NO: 307 - protein, 9 aa, Human immunodeficiency virus
FRKYTAFTI

SEQ ID NO: 308 - protein, 9 aa, Human immunodeficiency virus
RKYTAFTIP

SEQ ID NO: 309 - protein, 9 aa, Human immunodeficiency virus
KYTAFTIPS

SEQ ID NO: 310 - protein, 9 aa, Human immunodeficiency virus
NNETPGIRY

SEQ ID NO: 311 - protein, 9 aa, Human immunodeficiency virus
NETPGIRYQ

SEQ ID NO: 312 - protein, 9 aa, Human immunodeficiency virus
ETPGIRYQY

SEQ ID NO: 313 - protein, 9 aa, Human immunodeficiency virus
TPGIRYQYN

SEQ ID NO: 314 - protein, 9 aa, Human immunodeficiency virus
PGIRYQYNV

SEQ ID NO: 315 - protein, 9 aa, Human immunodeficiency virus
GIRYQYNVL

SEQ ID NO: 316 - protein, 9 aa, Human immunodeficiency virus
IRYQYNVLP

SEQ ID NO: 317 - protein, 9 aa, Human immunodeficiency virus
RYQYNVLPQ

SEQ ID NO: 318 - protein, 9 aa, Human immunodeficiency virus
YQYNVLPQG

SEQ ID NO: 319 - protein, 9 aa, Human immunodeficiency virus
QYNVLPQGW

SEQ ID NO: 320 - protein, 9 aa, Human immunodeficiency virus
YNVLPQGWK

SEQ ID NO: 321 - protein, 9 aa, Human immunodeficiency virus
NVLPQGWKG

SEQ ID NO: 322 - protein, 9 aa, Human immunodeficiency virus
VLPQGWKGS

SEQ ID NO: 323 - protein, 9 aa, Human immunodeficiency virus
LPQGWKGSP

SEQ ID NO: 324 - protein, 9 aa, Human immunodeficiency virus
PQGWKGSPA

SEQ ID NO: 325 - protein, 9 aa, Human immunodeficiency virus
QGWKGSPAI

SEQ ID NO: 326 - protein, 9 aa, Human immunodeficiency virus
GWKGSPAIF

SEQ ID NO: 327 - protein, 9 aa, Human immunodeficiency virus
WKGSPAIFQ

SEQ ID NO: 328 - protein, 9 aa, Human immunodeficiency virus
SMTKILEPF

SEQ ID NO: 329 - protein, 9 aa, Human immunodeficiency virus
MTKILEPFR

SEQ ID NO: 330 - protein, 9 aa, Human immunodeficiency virus
DDLYVGSDL

SEQ ID NO: 331 - protein, 9 aa, Human immunodeficiency virus
DLYVGSDLE

SEQ ID NO: 332 - protein, 9 aa, Human immunodeficiency virus
LYVGSDLEI

SEQ ID NO: 333 - protein, 9 aa, Human immunodeficiency virus
KHQKEPPFL

SEQ ID NO: 334 - protein, 9 aa, Human immunodeficiency virus
HQKEPPFLW

SEQ ID NO: 335 - protein, 9 aa, Human immunodeficiency virus
QKEPPFLWM

SEQ ID NO: 336 - protein, 9 aa, Human immunodeficiency virus
KEPPFLWMG

SEQ ID NO: 337 - protein, 9 aa, Human immunodeficiency virus
EPPFLWMGY

SEQ ID NO: 338 - protein, 9 aa, Human immunodeficiency virus
PPFLWMGYE

SEQ ID NO: 339 - protein, 9 aa, Human immunodeficiency virus
PFLWMGYEL

SEQ ID NO: 340 - protein, 9 aa, Human immunodeficiency virus
FLWMGYELH

SEQ ID NO: 341 - protein, 9 aa, Human immunodeficiency virus
LWMGYELHP

SEQ ID NO: 342 - protein, 9 aa, Human immunodeficiency virus
WMGYELHPD

SEQ ID NO: 343 - protein, 9 aa, Human immunodeficiency virus
MGYELHPDK

SEQ ID NO: 344 - protein, 9 aa, Human immunodeficiency virus
GYELHPDKW

SEQ ID NO: 345 - protein, 9 aa, Human immunodeficiency virus
YELHPDKWT

SEQ ID NO: 346 - protein, 9 aa, Human immunodeficiency virus
ELHPDKWTV

SEQ ID NO: 347 - protein, 9 aa, Human immunodeficiency virus
LHPDKWTVQ

SEQ ID NO: 348 - protein, 9 aa, Human immunodeficiency virus
HPDKWTVQP

SEQ ID NO: 349 - protein, 9 aa, Human immunodeficiency virus
PDKWTVQPI

SEQ ID NO: 350 - protein, 9 aa, Human immunodeficiency virus
LPEKDSWTV

SEQ ID NO: 351 - protein, 9 aa, Human immunodeficiency virus
PEKDSWTVN

SEQ ID NO: 352 - protein, 9 aa, Human immunodeficiency virus
EKDSWTVND

SEQ ID NO: 353 - protein, 9 aa, Human immunodeficiency virus
KDSWTVNDI

SEQ ID NO: 354 - protein, 9 aa, Human immunodeficiency virus
DSWTVNDIQ

SEQ ID NO: 355 - protein, 9 aa, Human immunodeficiency virus
SWTVNDIQK

SEQ ID NO: 356 - protein, 9 aa, Human immunodeficiency virus
WTVNDIQKL

SEQ ID NO: 357 - protein, 9 aa, Human immunodeficiency virus
TVNDIQKLV

SEQ ID NO: 358 - protein, 9 aa, Human immunodeficiency virus
VNDIQKLVG

SEQ ID NO: 359 - protein, 9 aa, Human immunodeficiency virus
NDIQKLVGK

SEQ ID NO: 360 - protein, 9 aa, Human immunodeficiency virus
DIQKLVGKL

SEQ ID NO: 361 - protein, 9 aa, Human immunodeficiency virus
IQKLVGKLN

SEQ ID NO: 362 - protein, 9 aa, Human immunodeficiency virus
QKLVGKLNW

SEQ ID NO: 363 - protein, 9 aa, Human immunodeficiency virus
KLVGKLNWA

SEQ ID NO: 364 - protein, 9 aa, Human immunodeficiency virus
LVGKLNWAS

SEQ ID NO: 365 - protein, 9 aa, Human immunodeficiency virus
VGKLNWASQ

SEQ ID NO: 366 - protein, 9 aa, Human immunodeficiency virus
GKLNWASQI

SEQ ID NO: 367 - protein, 9 aa, Human immunodeficiency virus
KLNWASQIY

SEQ ID NO: 368 - protein, 9 aa, Human immunodeficiency virus
EAELELAEN

SEQ ID NO: 369 - protein, 9 aa, Human immunodeficiency virus
AELELAENR

SEQ ID NO: 370 - protein, 9 aa, Human immunodeficiency virus
ELELAENRE

SEQ ID NO: 371 - protein, 9 aa, Human immunodeficiency virus
LELAENREI

SEQ ID NO: 372 - protein, 9 aa, Human immunodeficiency virus
ELAENREIL

SEQ ID NO: 373 - protein, 9 aa, Human immunodeficiency virus
LAENREILK

SEQ ID NO: 374 - protein, 9 aa, Human immunodeficiency virus
AENREILKE

SEQ ID NO: 375 - protein, 9 aa, Human immunodeficiency virus
ENREILKEP

SEQ ID NO: 376 - protein, 9 aa, Human immunodeficiency virus
NREILKEPV

SEQ ID NO: 377 - protein, 9 aa, Human immunodeficiency virus
REILKEPVH

SEQ ID NO: 378 - protein, 9 aa, Human immunodeficiency virus
EILKEPVHG

SEQ ID NO: 379 - protein, 9 aa, Human immunodeficiency virus
FLDGIDKAQ

SEQ ID NO: 380 - protein, 9 aa, Human immunodeficiency virus
EIVASCDKC

SEQ ID NO: 381 - protein, 9 aa, Human immunodeficiency virus
CDKCQLKGE

SEQ ID NO: 382 - protein, 9 aa, Human immunodeficiency virus
DKCQLKGEA

SEQ ID NO: 383 - protein, 9 aa, Human immunodeficiency virus
HGQVDCSPG

SEQ ID NO: 384 - protein, 9 aa, Human immunodeficiency virus
GQVDCSPGI

SEQ ID NO: 385 - protein, 9 aa, Human immunodeficiency virus
QVDCSPGIW

SEQ ID NO: 386 - protein, 9 aa, Human immunodeficiency virus
VDCSPGIWQ

SEQ ID NO: 387 - protein, 9 aa, Human immunodeficiency virus
DCSPGIWQL

SEQ ID NO: 388 - protein, 9 aa, Human immunodeficiency virus
CSPGIWQLD

SEQ ID NO: 389 - protein, 9 aa, Human immunodeficiency virus
SPGIWQLDC

SEQ ID NO: 390 - protein, 9 aa, Human immunodeficiency virus
PGIWQLDCT

SEQ ID NO: 391 - protein, 9 aa, Human immunodeficiency virus
GIWQLDCTH

SEQ ID NO: 392 - protein, 9 aa, Human immunodeficiency virus
IWQLDCTHL

SEQ ID NO: 393 - protein, 9 aa, Human immunodeficiency virus
WQLDCTHLE

SEQ ID NO: 394 - protein, 9 aa, Human immunodeficiency virus
QLDCTHLEG

SEQ ID NO: 395 - protein, 9 aa, Human immunodeficiency virus
LDCTHLEGK

SEQ ID NO: 396 - protein, 9 aa, Human immunodeficiency virus
ILVAVHVAS

SEQ ID NO: 397 - protein, 9 aa, Human immunodeficiency virus
LVAVHVASG

SEQ ID NO: 398 - protein, 9 aa, Human immunodeficiency virus
VAVHVASGY

SEQ ID NO: 399 - protein, 9 aa, Human immunodeficiency virus
AVHVASGYI

SEQ ID NO: 400 - protein, 9 aa, Human immunodeficiency virus
VHVASGYIE

SEQ ID NO: 401 - protein, 9 aa, Human immunodeficiency virus
HVASGYIEA

SEQ ID NO: 402 - protein, 9 aa, Human immunodeficiency virus
VASGYIEAE

SEQ ID NO: 403 - protein, 9 aa, Human immunodeficiency virus
ASGYIEAEV

SEQ ID NO: 404 - protein, 9 aa, Human immunodeficiency virus
SGYIEAEVI

SEQ ID NO: 405 - protein, 9 aa, Human immunodeficiency virus
GYIEAEVIP

SEQ ID NO: 406 - protein, 9 aa, Human immunodeficiency virus
YIEAEVIPA

SEQ ID NO: 407 - protein, 9 aa, Human immunodeficiency virus
IEAEVIPAE

SEQ ID NO: 408 - protein, 9 aa, Human immunodeficiency virus
EAEVIPAET

SEQ ID NO: 409 - protein, 9 aa, Human immunodeficiency virus
AEVIPAETG

SEQ ID NO: 410 - protein, 9 aa, Human immunodeficiency virus
EVIPAETGQ

SEQ ID NO: 411 - protein, 9 aa, Human immunodeficiency virus
VIPAETGQE

SEQ ID NO: 412 - protein, 9 aa, Human immunodeficiency virus
IPAETGQET

SEQ ID NO: 413 - protein, 9 aa, Human immunodeficiency virus
PAETGQETA

SEQ ID NO: 414 - protein, 9 aa, Human immunodeficiency virus
AETGQETAY

SEQ ID NO: 415 - protein, 9 aa, Human immunodeficiency virus
ETGQETAYF

SEQ ID NO: 416 - protein, 9 aa, Human immunodeficiency virus
LKLAGRWPV

SEQ ID NO: 417 - protein, 9 aa, Human immunodeficiency virus
KLAGRWPVK

SEQ ID NO: 418 - protein, 9 aa, Human immunodeficiency virus
VRAACWWAG

SEQ ID NO: 419 - protein, 9 aa, Human immunodeficiency virus
RAACWWAGI

SEQ ID NO: 420 - protein, 9 aa, Human immunodeficiency virus
ACWWAGIKQ

SEQ ID NO: 421 - protein, 9 aa, Human immunodeficiency virus
CWWAGIKQE

SEQ ID NO: 422 - protein, 9 aa, Human immunodeficiency virus
WWAGIKQEF

SEQ ID NO: 423 - protein, 9 aa, Human immunodeficiency virus
WAGIKQEFG

SEQ ID NO: 424 - protein, 9 aa, Human immunodeficiency virus
AGIKQEFGI

SEQ ID NO: 425 - protein, 9 aa, Human immunodeficiency virus
GIKQEFGIP

SEQ ID NO: 426 - protein, 9 aa, Human immunodeficiency virus
IKQEFGIPY

SEQ ID NO: 427 - protein, 9 aa, Human immunodeficiency virus
KQEFGIPYN

SEQ ID NO: 428 - protein, 9 aa, Human immunodeficiency virus
QEFGIPYNP

SEQ ID NO: 429 - protein, 9 aa, Human immunodeficiency virus
EFGIPYNPQ

SEQ ID NO: 430 - protein, 9 aa, Human immunodeficiency virus
FGIPYNPQS

SEQ ID NO: 431 - protein, 9 aa, Human immunodeficiency virus
GIPYNPQSQ

SEQ ID NO: 432 - protein, 9 aa, Human immunodeficiency virus
IPYNPQSQG

SEQ ID NO: 433 - protein, 9 aa, Human immunodeficiency virus
PYNPQSQGV

SEQ ID NO: 434 - protein, 9 aa, Human immunodeficiency virus
YNPQSQGVV

SEQ ID NO: 435 - protein, 9 aa, Human immunodeficiency virus
NPQSQGVVE

SEQ ID NO: 436 - protein, 9 aa, Human immunodeficiency virus
PQSQGVVES

SEQ ID NO: 437 - protein, 9 aa, Human immunodeficiency virus
QSQGVVESM

SEQ ID NO: 438 - protein, 9 aa, Human immunodeficiency virus
SQGVVESMN

SEQ ID NO: 439 - protein, 9 aa, Human immunodeficiency virus
ELKKIIGQV

SEQ ID NO: 440 - protein, 9 aa, Human immunodeficiency virus
LKKIIGQVR

SEQ ID NO: 441 - protein, 9 aa, Human immunodeficiency virus
IIGQVRDQA

SEQ ID NO: 442 - protein, 9 aa, Human immunodeficiency virus
IGQVRDQAE

SEQ ID NO: 443 - protein, 9 aa, Human immunodeficiency virus
GQVRDQAEH

SEQ ID NO: 444 - protein, 9 aa, Human immunodeficiency virus
QVRDQAEHL

SEQ ID NO: 445 - protein, 9 aa, Human immunodeficiency virus
VRDQAEHLK

SEQ ID NO: 446 - protein, 9 aa, Human immunodeficiency virus
RDQAEHLKT

SEQ ID NO: 447 - protein, 9 aa, Human immunodeficiency virus
DQAEHLKTA

SEQ ID NO: 448 - protein, 9 aa, Human immunodeficiency virus
QAEHLKTAV

SEQ ID NO: 449 - protein, 9 aa, Human immunodeficiency virus
AEHLKTAVQ

SEQ ID NO: 450 - protein, 9 aa, Human immunodeficiency virus
EHLKTAVQM

SEQ ID NO: 451 - protein, 9 aa, Human immunodeficiency virus
HLKTAVQMA

SEQ ID NO: 452 - protein, 9 aa, Human immunodeficiency virus
LKTAVQMAV

SEQ ID NO: 453 - protein, 9 aa, Human immunodeficiency virus
KTAVQMAVF

SEQ ID NO: 454 - protein, 9 aa, Human immunodeficiency virus
TAVQMAVFI

SEQ ID NO: 455 - protein, 9 aa, Human immunodeficiency virus
AVQMAVFIH

SEQ ID NO: 456 - protein, 9 aa, Human immunodeficiency virus
VQMAVFIHN

SEQ ID NO: 457 - protein, 9 aa, Human immunodeficiency virus
QMAVFIHNF

SEQ ID NO: 458 - protein, 9 aa, Human immunodeficiency virus
MAVFIHNFK

SEQ ID NO: 459 - protein, 9 aa, Human immunodeficiency virus
AVFIHNFKR

SEQ ID NO: 460 - protein, 9 aa, Human immunodeficiency virus
VFIHNFKRK

SEQ ID NO: 461 - protein, 9 aa, Human immunodeficiency virus
FIHNFKRKG

SEQ ID NO: 462 - protein, 9 aa, Human immunodeficiency virus
IHNFKRKGG

SEQ ID NO: 463 - protein, 9 aa, Human immunodeficiency virus
HNFKRKGGI

SEQ ID NO: 464 - protein, 9 aa, Human immunodeficiency virus
NFKRKGGIG

SEQ ID NO: 465 - protein, 9 aa, Human immunodeficiency virus
FKRKGGIGG

SEQ ID NO: 466 - protein, 9 aa, Human immunodeficiency virus
KRKGGIGGY

SEQ ID NO: 467 - protein, 9 aa, Human immunodeficiency virus
RKGGIGGYS

SEQ ID NO: 468 - protein, 9 aa, Human immunodeficiency virus
KGGIGGYSA

SEQ ID NO: 469 - protein, 9 aa, Human immunodeficiency virus
GGIGGYSAG

SEQ ID NO: 470 - protein, 9 aa, Human immunodeficiency virus
GIGGYSAGE

SEQ ID NO: 471 - protein, 9 aa, Human immunodeficiency virus
IGGYSAGER

SEQ ID NO: 472 - protein, 9 aa, Human immunodeficiency virus
GGYSAGERI

SEQ ID NO: 473 - protein, 9 aa, Human immunodeficiency virus
KIQNFRVYY

SEQ ID NO: 474 - protein, 9 aa, Human immunodeficiency virus
IQNFRVYYR

SEQ ID NO: 475 - protein, 9 aa, Human immunodeficiency virus
QNFRVYYRD

SEQ ID NO: 476 - protein, 9 aa, Human immunodeficiency virus
FRVYYRDSR

SEQ ID NO: 477 - protein, 9 aa, Human immunodeficiency virus
PLWKGPAKL

SEQ ID NO: 478 - protein, 9 aa, Human immunodeficiency virus
LWKGPAKLL

SEQ ID NO: 479 - protein, 9 aa, Human immunodeficiency virus
WKGPAKLLW

SEQ ID NO: 480 - protein, 9 aa, Human immunodeficiency virus
KGPAKLLWK

SEQ ID NO: 481 - protein, 9 aa, Human immunodeficiency virus
GPAKLLWKG

SEQ ID NO: 482 - protein, 9 aa, Human immunodeficiency virus
PAKLLWKGE

SEQ ID NO: 483 - protein, 9 aa, Human immunodeficiency virus
AKLLWKGEG

SEQ ID NO: 484 - protein, 9 aa, Human immunodeficiency virus
KLLWKGEGA

SEQ ID NO: 485 - protein, 9 aa, Human immunodeficiency virus
LLWKGEGAV

SEQ ID NO: 486 - protein, 9 aa, Human immunodeficiency virus
LWKGEGAVV

SEQ ID NO: 487 - protein, 9 aa, Human immunodeficiency virus
WKGEGAVVI

SEQ ID NO: 488 - protein, 9 aa, Human immunodeficiency virus
KGEGAVVIQ

SEQ ID NO: 489 - protein, 9 aa, Human immunodeficiency virus
GEGAVVIQD

SEQ ID NO: 490 - protein, 9 aa, Human immunodeficiency virus
EGAVVIQDN

SEQ ID NO: 491 - protein, 9 aa, Human immunodeficiency virus
GAVVIQDNS

SEQ ID NO: 492 - protein, 9 aa, Human immunodeficiency virus
KIIRDYGKQ

SEQ ID NO: 493 - protein, 9 aa, Human immunodeficiency virus
IIRDYGKQM

SEQ ID NO: 494 - protein, 9 aa, Human immunodeficiency virus
IRDYGKQMA

SEQ ID NO: 495 - protein, 9 aa, Human immunodeficiency virus
RDYGKQMAG

SEQ ID NO: 496 - protein, 9 aa, Human immunodeficiency virus
DYGKQMAGD

SEQ ID NO: 497 - protein, 9 aa, Human immunodeficiency virus
YGKQMAGDD

SEQ ID NO: 498 - protein, 9 aa, Human immunodeficiency virus
GKQMAGDDC

SEQ ID NO: 499 - protein, 9 aa, Human immunodeficiency virus
KQMAGDDCV

SEQ ID NO: 500 - protein, 9 aa, Human immunodeficiency virus
QMAGDDCVA

SEQ ID NO: 501 - protein, 9 aa, Human immunodeficiency virus
MENRWQVMI

SEQ ID NO: 502 - protein, 9 aa, Human immunodeficiency virus
ENRWQVMIV

SEQ ID NO: 503 - protein, 9 aa, Human immunodeficiency virus
NRWQVMIVW

SEQ ID NO: 504 - protein, 9 aa, Human immunodeficiency virus
RWQVMIVWQ

SEQ ID NO: 505 - protein, 9 aa, Human immunodeficiency virus
WQVMIVWQV

SEQ ID NO: 506 - protein, 9 aa, Human immunodeficiency virus
QVMIVWQVD

SEQ ID NO: 507 - protein, 9 aa, Human immunodeficiency virus
VMIVWQVDR

SEQ ID NO: 508 - protein, 9 aa, Human immunodeficiency virus
MIVWQVDRM

SEQ ID NO: 509 - protein, 9 aa, Human immunodeficiency virus
IVWQVDRMR

SEQ ID NO: 510 - protein, 9 aa, Human immunodeficiency virus
VWQVDRMRI

SEQ ID NO: 511 - protein, 9 aa, Human immunodeficiency virus
SSEVHIPLG

SEQ ID NO: 512 - protein, 9 aa, Human immunodeficiency virus
TYWGLHTGE

SEQ ID NO: 513 - protein, 9 aa, Human immunodeficiency virus
YWGLHTGER

SEQ ID NO: 514 - protein, 9 aa, Human immunodeficiency virus
WHLGQGVSI

SEQ ID NO: 515 - protein, 9 aa, Human immunodeficiency virus
HLGQGVSIE

SEQ ID NO: 516 - protein, 9 aa, Human immunodeficiency virus
LGQGVSIEW

SEQ ID NO: 517 - protein, 9 aa, Human immunodeficiency virus
GQGVSIEWR

SEQ ID NO: 518 - protein, 9 aa, Human immunodeficiency virus
GHNKVGSLQ

SEQ ID NO: 519 - protein, 9 aa, Human immunodeficiency virus
HNKVGSLQY

SEQ ID NO: 520 - protein, 9 aa, Human immunodeficiency virus
NKVGSLQYL

SEQ ID NO: 521 - protein, 9 aa, Human immunodeficiency virus
KVGSLQYLA

SEQ ID NO: 522 - protein, 9 aa, Human immunodeficiency virus
VGSLQYLAL

SEQ ID NO: 523 - protein, 9 aa, Human immunodeficiency virus
KLTEDRWNK

SEQ ID NO: 524 - protein, 9 aa, Human immunodeficiency virus
LTEDRWNKP

SEQ ID NO: 525 - protein, 9 aa, Human immunodeficiency virus
TEDRWNKPQ

SEQ ID NO: 526 - protein, 9 aa, Human immunodeficiency virus
MEQAPEDQG

SEQ ID NO: 527 - protein, 9 aa, Human immunodeficiency virus
EQAPEDQGP

SEQ ID NO: 528 - protein, 9 aa, Human immunodeficiency virus
QAPEDQGPQ

SEQ ID NO: 529 - protein, 9 aa, Human immunodeficiency virus
APEDQGPQR

SEQ ID NO: 530 - protein, 9 aa, Human immunodeficiency virus
PEDQGPQRE

SEQ ID NO: 531 - protein, 9 aa, Human immunodeficiency virus
EDQGPQREP

SEQ ID NO: 532 - protein, 9 aa, Human immunodeficiency virus
WTLELLEEL

SEQ ID NO: 533 - protein, 9 aa, Human immunodeficiency virus
TLELLEELK

SEQ ID NO: 534 - protein, 9 aa, Human immunodeficiency virus
LEPWKHPGS

SEQ ID NO: 535 - protein, 9 aa, Human immunodeficiency virus
EPWKHPGSQ

SEQ ID NO: 536 - protein, 9 aa, Human immunodeficiency virus
PWKHPGSQP

SEQ ID NO: 537 - protein, 9 aa, Human immunodeficiency virus
LGISYGRKK

SEQ ID NO: 538 - protein, 9 aa, Human immunodeficiency virus
GISYGRKKR

SEQ ID NO: 539 - protein, 9 aa, Human immunodeficiency virus
ISYGRKKRR

SEQ ID NO: 540 - protein, 9 aa, Human immunodeficiency virus
SYGRKKRRQ

SEQ ID NO: 541 - protein, 9 aa, Human immunodeficiency virus
YGRKKRRQR

SEQ ID NO: 542 - protein, 9 aa, Human immunodeficiency virus
GRKKRRQRR

SEQ ID NO: 543 - protein, 9 aa, Human immunodeficiency virus
RKKRRQRRR

SEQ ID NO: 544 - protein, 9 aa, Human immunodeficiency virus
EGTRQARRN

SEQ ID NO: 545 - protein, 9 aa, Human immunodeficiency virus
GTRQARRNR

SEQ ID NO: 546 - protein, 9 aa, Human immunodeficiency virus
TRQARRNRR

SEQ ID NO: 547 - protein, 9 aa, Human immunodeficiency virus
RQARRNRRR

SEQ ID NO: 548 - protein, 9 aa, Human immunodeficiency virus
QARRNRRRR

SEQ ID NO: 549 - protein, 9 aa, Human immunodeficiency virus
ARRNRRRRW

SEQ ID NO: 550 - protein, 9 aa, Human immunodeficiency virus
RRNRRRRWR

SEQ ID NO: 551 - protein, 9 aa, Human immunodeficiency virus
ERAEDSGNE

SEQ ID NO: 552 - protein, 9 aa, Human immunodeficiency virus
RAEDSGNES

SEQ ID NO: 553 - protein, 9 aa, Human immunodeficiency virus
LWVTVYYGV

SEQ ID NO: 554 - protein, 9 aa, Human immunodeficiency virus
WVTVYYGVP

SEQ ID NO: 555 - protein, 9 aa, Human immunodeficiency virus
VTVYYGVPV

SEQ ID NO: 556 - protein, 9 aa, Human immunodeficiency virus
TVYYGVPVW

SEQ ID NO: 557 - protein, 9 aa, Human immunodeficiency virus
VYYGVPVWK

SEQ ID NO: 558 - protein, 9 aa, Human immunodeficiency virus
YYGVPVWKE

SEQ ID NO: 559 - protein, 9 aa, Human immunodeficiency virus
YGVPVWKEA

SEQ ID NO: 560 - protein, 9 aa, Human immunodeficiency virus
GVPVWKEAT

SEQ ID NO: 561 - protein, 9 aa, Human immunodeficiency virus
VPVWKEATT

SEQ ID NO: 562 - protein, 9 aa, Human immunodeficiency virus
PVWKEATTT

SEQ ID NO: 563 - protein, 9 aa, Human immunodeficiency virus
VWKEATTTL

SEQ ID NO: 564 - protein, 9 aa, Human immunodeficiency virus
WKEATTTLF

SEQ ID NO: 565 - protein, 9 aa, Human immunodeficiency virus
KEATTTLFC

SEQ ID NO: 566 - protein, 9 aa, Human immunodeficiency virus
EATTTLFCA

SEQ ID NO: 567 - protein, 9 aa, Human immunodeficiency virus
ATTTLFCAS

SEQ ID NO: 568 - protein, 9 aa, Human immunodeficiency virus
TTTLFCASD

SEQ ID NO: 569 - protein, 9 aa, Human immunodeficiency virus
TTLFCASDA

SEQ ID NO: 570 - protein, 9 aa, Human immunodeficiency virus
TLFCASDAK

SEQ ID NO: 571 - protein, 9 aa, Human immunodeficiency virus
HNVWATHAC

SEQ ID NO: 572 - protein, 9 aa, Human immunodeficiency virus
NVWATHACV

SEQ ID NO: 573 - protein, 9 aa, Human immunodeficiency virus
VWATHACVP

SEQ ID NO: 574 - protein, 9 aa, Human immunodeficiency virus
WATHACVPT

SEQ ID NO: 575 - protein, 9 aa, Human immunodeficiency virus
SLKPCVKLT

SEQ ID NO: 576 - protein, 9 aa, Human immunodeficiency virus
LKPCVKLTP

SEQ ID NO: 577 - protein, 9 aa, Human immunodeficiency virus
KPCVKLTPL

SEQ ID NO: 578 - protein, 9 aa, Human immunodeficiency virus
PCVKLTPLC

SEQ ID NO: 579 - protein, 9 aa, Human immunodeficiency virus
CVKLTPLCV

SEQ ID NO: 580 - protein, 9 aa, Human immunodeficiency virus
VKLTPLCVS

SEQ ID NO: 581 - protein, 9 aa, Human immunodeficiency virus
KLTPLCVSL

SEQ ID NO: 582 - protein, 9 aa, Human immunodeficiency virus
NVSTVQCTH

SEQ ID NO: 583 - protein, 9 aa, Human immunodeficiency virus
VSTVQCTHG

SEQ ID NO: 584 - protein, 9 aa, Human immunodeficiency virus
STVQCTHGI

SEQ ID NO: 585 - protein, 9 aa, Human immunodeficiency virus
PVVSTQLLL

SEQ ID NO: 586 - protein, 9 aa, Human immunodeficiency virus
VVSTQLLLN

SEQ ID NO: 587 - protein, 9 aa, Human immunodeficiency virus
VSTQLLLNG

SEQ ID NO: 588 - protein, 9 aa, Human immunodeficiency virus
STQLLLNGS

SEQ ID NO: 589 - protein, 9 aa, Human immunodeficiency virus
TQLLLNGSL

SEQ ID NO: 590 - protein, 9 aa, Human immunodeficiency virus
QLLLNGSLA

SEQ ID NO: 591 - protein, 9 aa, Human immunodeficiency virus
LLLNGSLAE

SEQ ID NO: 592 - protein, 9 aa, Human immunodeficiency virus
VGKAMYAPP

SEQ ID NO: 593 - protein, 9 aa, Human immunodeficiency virus
GKAMYAPPI

SEQ ID NO: 594 - protein, 9 aa, Human immunodeficiency virus
DNWRSELYK

SEQ ID NO: 595 - protein, 9 aa, Human immunodeficiency virus
NWRSELYKY

SEQ ID NO: 596 - protein, 9 aa, Human immunodeficiency virus
WRSELYKYK

SEQ ID NO: 597 - protein, 9 aa, Human immunodeficiency virus
RSELYKYKV

SEQ ID NO: 598 - protein, 9 aa, Human immunodeficiency virus
SELYKYKVV

SEQ ID NO: 599 - protein, 9 aa, Human immunodeficiency virus
AKRRVVQRE

SEQ ID NO: 600 - protein, 9 aa, Human immunodeficiency virus
FLGFLGAAG

SEQ ID NO: 601 - protein, 9 aa, Human immunodeficiency virus
LGFLGAAGS

SEQ ID NO: 602 - protein, 9 aa, Human immunodeficiency virus
GFLGAAGST

SEQ ID NO: 603 - protein, 9 aa, Human immunodeficiency virus
FLGAAGSTM

SEQ ID NO: 604 - protein, 9 aa, Human immunodeficiency virus
LGAAGSTMG

SEQ ID NO: 605 - protein, 9 aa, Human immunodeficiency virus
GAAGSTMGA

SEQ ID NO: 606 - protein, 9 aa, Human immunodeficiency virus
AAGSTMGAA

SEQ ID NO: 607 - protein, 9 aa, Human immunodeficiency virus
AGSTMGAAS

SEQ ID NO: 608 - protein, 9 aa, Human immunodeficiency virus
LLSGIVQQQ

SEQ ID NO: 609 - protein, 9 aa, Human immunodeficiency virus
LQLTVWGIK

SEQ ID NO: 610 - protein, 9 aa, Human immunodeficiency virus
QLTVWGIKQ

SEQ ID NO: 611 - protein, 9 aa, Human immunodeficiency virus
LTVWGIKQL

SEQ ID NO: 612 - protein, 9 aa, Human immunodeficiency virus
TVWGIKQLQ

SEQ ID NO: 613 - protein, 9 aa, Human immunodeficiency virus
VWGIKQLQA

SEQ ID NO: 614 - protein, 9 aa, Human immunodeficiency virus
WGIKQLQAR

SEQ ID NO: 615 - protein, 9 aa, Human immunodeficiency virus
GIKQLQARI

SEQ ID NO: 616 - protein, 9 aa, Human immunodeficiency virus
IKQLQARIL

SEQ ID NO: 617 - protein, 9 aa, Human immunodeficiency virus
KQLQARILA

SEQ ID NO: 618 - protein, 9 aa, Human immunodeficiency virus
DQQLLGIWG

SEQ ID NO: 619 - protein, 9 aa, Human immunodeficiency virus
QQLLGIWGC

SEQ ID NO: 620 - protein, 9 aa, Human immunodeficiency virus
QLLGIWGCS

SEQ ID NO: 621 - protein, 9 aa, Human immunodeficiency virus
LLGIWGCSG

SEQ ID NO: 622 - protein, 9 aa, Human immunodeficiency virus
LGIWGCSGK

SEQ ID NO: 623 - protein, 9 aa, Human immunodeficiency virus
GIWGCSGKL

SEQ ID NO: 624 - protein, 9 aa, Human immunodeficiency virus
IWGCSGKLI

SEQ ID NO: 625 - protein, 9 aa, Human immunodeficiency virus
WGCSGKLIC

SEQ ID NO: 626 - protein, 9 aa, Human immunodeficiency virus
GCSGKLICT

SEQ ID NO: 627 - protein, 9 aa, Human immunodeficiency virus
CSGKLICTT

SEQ ID NO: 628 - protein, 9 aa, Human immunodeficiency virus
WLWYIKLFI

SEQ ID NO: 629 - protein, 9 aa, Human immunodeficiency virus
AIAVAEGTD

SEQ ID NO: 630 - protein, 9 aa, Human immunodeficiency virus
IAVAEGTDR

SEQ ID NO: 631 - protein, 9 aa, Human immunodeficiency virus
PQVPLRPMT

SEQ ID NO: 632 - protein, 9 aa, Human immunodeficiency virus
FPDWQNYTP

SEQ ID NO: 633 - protein, 9 aa, Human immunodeficiency virus
PDWQNYTPG

SEQ ID NO: 634 - protein, 9 aa, Human immunodeficiency virus
DWQNYTPGP

SEQ ID NO: 635 - protein, 9 aa, Human immunodeficiency virus
WQNYTPGPG

SEQ ID NO: 636 - protein, 9 aa, Human immunodeficiency virus
FGWCYKLVP

SEQ ID NO: 637 - protein, 9 aa, Human immunodeficiency virus
MGARASVLS

SEQ ID NO: 638 - protein, 9 aa, Human immunodeficiency virus
GARASVLSG

SEQ ID NO: 639 - protein, 9 aa, Human immunodeficiency virus
ARASVLSGG

SEQ ID NO: 640 - protein, 9 aa, Human immunodeficiency virus
WEKIRLRPG

SEQ ID NO: 641 - protein, 9 aa, Human immunodeficiency virus
EKIRLRPGG

SEQ ID NO: 642 - protein, 9 aa, Human immunodeficiency virus
VWASRELER

SEQ ID NO: 643 - protein, 9 aa, Human immunodeficiency virus
WASRELERF

SEQ ID NO: 644 - protein, 9 aa, Human immunodeficiency virus
ASRELERFA

SEQ ID NO: 645 - protein, 9 aa, Human immunodeficiency virus
SQNYPIVQN

SEQ ID NO: 646 - protein, 9 aa, Human immunodeficiency virus
SPRTLNAWV

SEQ ID NO: 647 - protein, 9 aa, Human immunodeficiency virus
PRTLNAWVK

SEQ ID NO: 648 - protein, 9 aa, Human immunodeficiency virus
RTLNAWVKV

SEQ ID NO: 649 - protein, 9 aa, Human immunodeficiency virus
EEKAFSPEV

SEQ ID NO: 650 - protein, 9 aa, Human immunodeficiency virus
EKAFSPEVI

SEQ ID NO: 651 - protein, 9 aa, Human immunodeficiency virus
KAFSPEVIP

SEQ ID NO: 652 - protein, 9 aa, Human immunodeficiency virus
AFSPEVIPM

SEQ ID NO: 653 - protein, 9 aa, Human immunodeficiency virus
FSPEVIPMF

SEQ ID NO: 654 - protein, 9 aa, Human immunodeficiency virus
ALSEGATPQ

SEQ ID NO: 655 - protein, 9 aa, Human immunodeficiency virus
LSEGATPQD

SEQ ID NO: 656 - protein, 9 aa, Human immunodeficiency virus
SEGATPQDL

SEQ ID NO: 657 - protein, 9 aa, Human immunodeficiency virus
EGATPQDLN

SEQ ID NO: 658 - protein, 9 aa, Human immunodeficiency virus
GATPQDLNT

SEQ ID NO: 659 - protein, 9 aa, Human immunodeficiency virus
ATPQDLNTM

SEQ ID NO: 660 - protein, 9 aa, Human immunodeficiency virus
TPQDLNTML

SEQ ID NO: 661 - protein, 9 aa, Human immunodeficiency virus
PQDLNTMLN

SEQ ID NO: 662 - protein, 9 aa, Human immunodeficiency virus
QDLNTMLNT

SEQ ID NO: 663 - protein, 9 aa, Human immunodeficiency virus
DLNTMLNTV

SEQ ID NO: 664 - protein, 9 aa, Human immunodeficiency virus
LNTMLNTVG

SEQ ID NO: 665 - protein, 9 aa, Human immunodeficiency virus
NTMLNTVGG

SEQ ID NO: 666 - protein, 9 aa, Human immunodeficiency virus
TMLNTVGGH

SEQ ID NO: 667 - protein, 9 aa, Human immunodeficiency virus
MLNTVGGHQ

SEQ ID NO: 668 - protein, 9 aa, Human immunodeficiency virus
LNTVGGHQA

SEQ ID NO: 669 - protein, 9 aa, Human immunodeficiency virus
NTVGGHQAA

SEQ ID NO: 670 - protein, 9 aa, Human immunodeficiency virus
TVGGHQAAM

SEQ ID NO: 671 - protein, 9 aa, Human immunodeficiency virus
VGGHQAAMQ

SEQ ID NO: 672 - protein, 9 aa, Human immunodeficiency virus
GGHQAAMQM

SEQ ID NO: 673 - protein, 9 aa, Human immunodeficiency virus
GHQAAMQML

SEQ ID NO: 674 - protein, 9 aa, Human immunodeficiency virus
HQAAMQMLK

SEQ ID NO: 675 - protein, 9 aa, Human immunodeficiency virus
TINEEAAEW

SEQ ID NO: 676 - protein, 9 aa, Human immunodeficiency virus
INEEAAEWD

SEQ ID NO: 677 - protein, 9 aa, Human immunodeficiency virus
NEEAAEWDR

SEQ ID NO: 678 - protein, 9 aa, Human immunodeficiency virus
PGQMREPRG

SEQ ID NO: 679 - protein, 9 aa, Human immunodeficiency virus
GQMREPRGS

SEQ ID NO: 680 - protein, 9 aa, Human immunodeficiency virus
QMREPRGSD

SEQ ID NO: 681 - protein, 9 aa, Human immunodeficiency virus
MREPRGSDI

SEQ ID NO: 682 - protein, 9 aa, Human immunodeficiency virus
REPRGSDIA

SEQ ID NO: 683 - protein, 9 aa, Human immunodeficiency virus
EPRGSDIAG

SEQ ID NO: 684 - protein, 9 aa, Human immunodeficiency virus
PRGSDIAGT

SEQ ID NO: 685 - protein, 9 aa, Human immunodeficiency virus
RGSDIAGTT

SEQ ID NO: 686 - protein, 9 aa, Human immunodeficiency virus
GSDIAGTTS

SEQ ID NO: 687 - protein, 9 aa, Human immunodeficiency virus
SDIAGTTST

SEQ ID NO: 688 - protein, 9 aa, Human immunodeficiency virus
DIAGTTSTL

SEQ ID NO: 689 - protein, 9 aa, Human immunodeficiency virus
IAGTTSTLQ

SEQ ID NO: 690 - protein, 9 aa, Human immunodeficiency virus
AGTTSTLQE

SEQ ID NO: 691 - protein, 9 aa, Human immunodeficiency virus
GTTSTLQEQ

SEQ ID NO: 692 - protein, 9 aa, Human immunodeficiency virus
TTSTLQEQI

SEQ ID NO: 693 - protein, 9 aa, Human immunodeficiency virus
NPPIPVGEI

SEQ ID NO: 694 - protein, 9 aa, Human immunodeficiency virus
PPIPVGEIY

SEQ ID NO: 695 - protein, 9 aa, Human immunodeficiency virus
PIPVGEIYK

SEQ ID NO: 696 - protein, 9 aa, Human immunodeficiency virus
GLNKIVRMY

SEQ ID NO: 697 - protein, 9 aa, Human immunodeficiency virus
LNKIVRMYS

SEQ ID NO: 698 - protein, 9 aa, Human immunodeficiency virus
NKIVRMYSP

SEQ ID NO: 699 - protein, 9 aa, Human immunodeficiency virus
QGPKEPFRD

SEQ ID NO: 700 - protein, 9 aa, Human immunodeficiency virus
GPKEPFRDY

SEQ ID NO: 701 - protein, 9 aa, Human immunodeficiency virus
PKEPFRDYV

SEQ ID NO: 702 - protein, 9 aa, Human immunodeficiency virus
KEPFRDYVD

SEQ ID NO: 703 - protein, 9 aa, Human immunodeficiency virus
EPFRDYVDR

SEQ ID NO: 704 - protein, 9 aa, Human immunodeficiency virus
PFRDYVDRF

SEQ ID NO: 705 - protein, 9 aa, Human immunodeficiency virus
FRDYVDRFY

SEQ ID NO: 706 - protein, 9 aa, Human immunodeficiency virus
RDYVDRFYK

SEQ ID NO: 707 - protein, 9 aa, Human immunodeficiency virus
DYVDRFYKT

SEQ ID NO: 708 - protein, 9 aa, Human immunodeficiency virus
YVDRFYKTL

SEQ ID NO: 709 - protein, 9 aa, Human immunodeficiency virus
VDRFYKTLR

SEQ ID NO: 710 - protein, 9 aa, Human immunodeficiency virus
DRFYKTLRA

SEQ ID NO: 711 - protein, 9 aa, Human immunodeficiency virus
RFYKTLRAE

SEQ ID NO: 712 - protein, 9 aa, Human immunodeficiency virus
FYKTLRAEQ

SEQ ID NO: 713 - protein, 9 aa, Human immunodeficiency virus
YKTLRAEQA

SEQ ID NO: 714 - protein, 9 aa, Human immunodeficiency virus
VKNWMTETL

SEQ ID NO: 715 - protein, 9 aa, Human immunodeficiency virus
KNWMTETLL

SEQ ID NO: 716 - protein, 9 aa, Human immunodeficiency virus
NWMTETLLV

SEQ ID NO: 717 - protein, 9 aa, Human immunodeficiency virus
WMTETLLVQ

SEQ ID NO: 718 - protein, 9 aa, Human immunodeficiency virus
MTETLLVQN

SEQ ID NO: 719 - protein, 9 aa, Human immunodeficiency virus
TETLLVQNA

SEQ ID NO: 720 - protein, 9 aa, Human immunodeficiency virus
ETLLVQNAN

SEQ ID NO: 721 - protein, 9 aa, Human immunodeficiency virus
TLLVQNANP

SEQ ID NO: 722 - protein, 9 aa, Human immunodeficiency virus
LLVQNANPD

SEQ ID NO: 723 - protein, 9 aa, Human immunodeficiency virus
LVQNANPDC

SEQ ID NO: 724 - protein, 9 aa, Human immunodeficiency virus
VQNANPDCK

SEQ ID NO: 725 - protein, 9 aa, Human immunodeficiency virus
QNANPDCKT

SEQ ID NO: 726 - protein, 9 aa, Human immunodeficiency virus
NANPDCKTI

SEQ ID NO: 727 - protein, 9 aa, Human immunodeficiency virus
ANPDCKTIL

SEQ ID NO: 728 - protein, 9 aa, Human immunodeficiency virus
NPDCKTILK

SEQ ID NO: 729 - protein, 9 aa, Human immunodeficiency virus
PDCKTILKA

SEQ ID NO: 730 - protein, 9 aa, Human immunodeficiency virus
DCKTILKAL

SEQ ID NO: 731 - protein, 9 aa, Human immunodeficiency virus
CKTILKALG

SEQ ID NO: 732 - protein, 9 aa, Human immunodeficiency virus
KTILKALGP

SEQ ID NO: 733 - protein, 9 aa, Human immunodeficiency virus
ATLEEMMTA

SEQ ID NO: 734 - protein, 9 aa, Human immunodeficiency virus
TLEEMMTAC

SEQ ID NO: 735 - protein, 9 aa, Human immunodeficiency virus
LEEMMTACQ

SEQ ID NO: 736 - protein, 9 aa, Human immunodeficiency virus
EEMMTACQG

SEQ ID NO: 737 - protein, 9 aa, Human immunodeficiency virus
EMMTACQGV

SEQ ID NO: 738 - protein, 9 aa, Human immunodeficiency virus
MMTACQGVG

SEQ ID NO: 739 - protein, 9 aa, Human immunodeficiency virus
MTACQGVGG

SEQ ID NO: 740 - protein, 9 aa, Human immunodeficiency virus
TACQGVGGP

SEQ ID NO: 741 - protein, 9 aa, Human immunodeficiency virus
HKARVLAEA

SEQ ID NO: 742 - protein, 9 aa, Human immunodeficiency virus
KARVLAEAM

SEQ ID NO: 743 - protein, 9 aa, Human immunodeficiency virus
ARVLAEAMS

SEQ ID NO: 744 - protein, 9 aa, Human immunodeficiency virus
KCFNCGKEG

SEQ ID NO: 745 - protein, 9 aa, Human immunodeficiency virus
CFNCGKEGH

SEQ ID NO: 746 - protein, 9 aa, Human immunodeficiency virus
NFLGKIWPS

SEQ ID NO: 747 - protein, 9 aa, Human immunodeficiency virus
KGRPGNFLQ

SEQ ID NO: 748 - protein, 9 aa, Human immunodeficiency virus
PQITLWQRP

SEQ ID NO: 749 - protein, 9 aa, Human immunodeficiency virus
KEALLDTGA

SEQ ID NO: 750 - protein, 9 aa, Human immunodeficiency virus
EALLDTGAD

SEQ ID NO: 751 - protein, 9 aa, Human immunodeficiency virus
ALLDTGADD

SEQ ID NO: 752 - protein, 9 aa, Human immunodeficiency virus
LLDTGADDT

SEQ ID NO: 753 - protein, 9 aa, Human immunodeficiency virus
LDTGADDTV

SEQ ID NO: 754 - protein, 9 aa, Human immunodeficiency virus
DTGADDTVL

SEQ ID NO: 755 - protein, 9 aa, Human immunodeficiency virus
TGADDTVLE

SEQ ID NO: 756 - protein, 9 aa, Human immunodeficiency virus
PKMIGGIGG

SEQ ID NO: 757 - protein, 9 aa, Human immunodeficiency virus
KMIGGIGGF

SEQ ID NO: 758 - protein, 9 aa, Human immunodeficiency virus
IGGIGGFIK

SEQ ID NO: 759 - protein, 9 aa, Human immunodeficiency virus
GGIGGFIKV

SEQ ID NO: 760 - protein, 9 aa, Human immunodeficiency virus
GCTLNFPIS

SEQ ID NO: 761 - protein, 9 aa, Human immunodeficiency virus
CTLNFPISP

SEQ ID NO: 762 - protein, 9 aa, Human immunodeficiency virus
TLNFPISPI

SEQ ID NO: 763 - protein, 9 aa, Human immunodeficiency virus
LNFPISPIE

SEQ ID NO: 764 - protein, 9 aa, Human immunodeficiency virus
NFPISPIET

SEQ ID NO: 765 - protein, 9 aa, Human immunodeficiency virus
FPISPIETV

SEQ ID NO: 766 - protein, 9 aa, Human immunodeficiency virus
PISPIETVP

SEQ ID NO: 767 - protein, 9 aa, Human immunodeficiency virus
ISPIETVPV

SEQ ID NO: 768 - protein, 9 aa, Human immunodeficiency virus
SPIETVPVK

SEQ ID NO: 769 - protein, 9 aa, Human immunodeficiency virus
PIETVPVKL

SEQ ID NO: 770 - protein, 9 aa, Human immunodeficiency virus
IETVPVKLK

SEQ ID NO: 771 - protein, 9 aa, Human immunodeficiency virus
ETVPVKLKP

SEQ ID NO: 772 - protein, 9 aa, Human immunodeficiency virus
TVPVKLKPG

SEQ ID NO: 773 - protein, 9 aa, Human immunodeficiency virus
VPVKLKPGM

SEQ ID NO: 774 - protein, 9 aa, Human immunodeficiency virus
PVKLKPGMD

SEQ ID NO: 775 - protein, 9 aa, Human immunodeficiency virus
VKLKPGMDG

SEQ ID NO: 776 - protein, 9 aa, Human immunodeficiency virus
KLKPGMDGP

SEQ ID NO: 777 - protein, 9 aa, Human immunodeficiency virus
VKQWPLTEE

SEQ ID NO: 778 - protein, 9 aa, Human immunodeficiency virus
KQWPLTEEK

SEQ ID NO: 779 - protein, 9 aa, Human immunodeficiency virus
QWPLTEEKI

SEQ ID NO: 780 - protein, 9 aa, Human immunodeficiency virus
WPLTEEKIK

SEQ ID NO: 781 - protein, 9 aa, Human immunodeficiency virus
PLTEEKIKA

SEQ ID NO: 782 - protein, 9 aa, Human immunodeficiency virus
LTEEKIKAL

SEQ ID NO: 783 - protein, 9 aa, Human immunodeficiency virus
GKISKIGPE

SEQ ID NO: 784 - protein, 9 aa, Human immunodeficiency virus
KISKIGPEN

SEQ ID NO: 785 - protein, 9 aa, Human immunodeficiency virus
ISKIGPENP

SEQ ID NO: 786 - protein, 9 aa, Human immunodeficiency virus
SKIGPENPY

SEQ ID NO: 787 - protein, 9 aa, Human immunodeficiency virus
KIGPENPYN

SEQ ID NO: 788 - protein, 9 aa, Human immunodeficiency virus
IGPENPYNT

SEQ ID NO: 789 - protein, 9 aa, Human immunodeficiency virus
GPENPYNTP

SEQ ID NO: 790 - protein, 9 aa, Human immunodeficiency virus
WRKLVDFRE

SEQ ID NO: 791 - protein, 9 aa, Human immunodeficiency virus
RKLVDFREL

SEQ ID NO: 792 - protein, 9 aa, Human immunodeficiency virus
KLVDFRELN

SEQ ID NO: 793 - protein, 9 aa, Human immunodeficiency virus
LVDFRELNK

SEQ ID NO: 794 - protein, 9 aa, Human immunodeficiency virus
TQDFWEVQL

SEQ ID NO: 795 - protein, 9 aa, Human immunodeficiency virus
QDFWEVQLG

SEQ ID NO: 796 - protein, 9 aa, Human immunodeficiency virus
DFWEVQLGI

SEQ ID NO: 797 - protein, 9 aa, Human immunodeficiency virus
FWEVQLGIP

SEQ ID NO: 798 - protein, 9 aa, Human immunodeficiency virus
WEVQLGIPH

SEQ ID NO: 799 - protein, 9 aa, Human immunodeficiency virus
EVQLGIPHP

SEQ ID NO: 800 - protein, 9 aa, Human immunodeficiency virus
VQLGIPHPA

SEQ ID NO: 801 - protein, 9 aa, Human immunodeficiency virus
QLGIPHPAG

SEQ ID NO: 802 - protein, 9 aa, Human immunodeficiency virus
LGIPHPAGL

SEQ ID NO: 803 - protein, 9 aa, Human immunodeficiency virus
GIPHPAGLK

SEQ ID NO: 804 - protein, 9 aa, Human immunodeficiency virus
IPHPAGLKK

SEQ ID NO: 805 - protein, 9 aa, Human immunodeficiency virus
KSVTVLDVG

SEQ ID NO: 806 - protein, 9 aa, Human immunodeficiency virus
SVTVLDVGD

SEQ ID NO: 807 - protein, 9 aa, Human immunodeficiency virus
VTVLDVGDA

SEQ ID NO: 808 - protein, 9 aa, Human immunodeficiency virus
TVLDVGDAY

SEQ ID NO: 809 - protein, 9 aa, Human immunodeficiency virus
VLDVGDAYF

SEQ ID NO: 810 - protein, 9 aa, Human immunodeficiency virus
LDVGDAYFS

SEQ ID NO: 811 - protein, 9 aa, Human immunodeficiency virus
FRKYTAFTI

SEQ ID NO: 812 - protein, 9 aa, Human immunodeficiency virus
RKYTAFTIP

SEQ ID NO: 813 - protein, 9 aa, Human immunodeficiency virus
KYTAFTIPS

SEQ ID NO: 814 - protein, 9 aa, Human immunodeficiency virus
NNETPGIRY

SEQ ID NO: 815 - protein, 9 aa, Human immunodeficiency virus
NETPGIRYQ

SEQ ID NO: 816 - protein, 9 aa, Human immunodeficiency virus
ETPGIRYQY

SEQ ID NO: 817 - protein, 9 aa, Human immunodeficiency virus
TPGIRYQYN

SEQ ID NO: 818 - protein, 9 aa, Human immunodeficiency virus
PGIRYQYNV

SEQ ID NO: 819 - protein, 9 aa, Human immunodeficiency virus
GIRYQYNVL

SEQ ID NO: 820 - protein, 9 aa, Human immunodeficiency virus
IRYQYNVLP

SEQ ID NO: 821 - protein, 9 aa, Human immunodeficiency virus
RYQYNVLPQ

SEQ ID NO: 822 - protein, 9 aa, Human immunodeficiency virus
YQYNVLPQG

SEQ ID NO: 823 - protein, 9 aa, Human immunodeficiency virus
QYNVLPQGW

SEQ ID NO: 824 - protein, 9 aa, Human immunodeficiency virus
YNVLPQGWK

SEQ ID NO: 825 - protein, 9 aa, Human immunodeficiency virus
NVLPQGWKG

SEQ ID NO: 826 - protein, 9 aa, Human immunodeficiency virus
VLPQGWKGS

SEQ ID NO: 827 - protein, 9 aa, Human immunodeficiency virus
LPQGWKGSP

SEQ ID NO: 828 - protein, 9 aa, Human immunodeficiency virus
PQGWKGSPA

SEQ ID NO: 829 - protein, 9 aa, Human immunodeficiency virus
QGWKGSPAI

SEQ ID NO: 830 - protein, 9 aa, Human immunodeficiency virus
GWKGSPAIF

SEQ ID NO: 831 - protein, 9 aa, Human immunodeficiency virus
WKGSPAIFQ

SEQ ID NO: 832 - protein, 9 aa, Human immunodeficiency virus
SMTKILEPF

SEQ ID NO: 833 - protein, 9 aa, Human immunodeficiency virus
MTKILEPFR

SEQ ID NO: 834 - protein, 9 aa, Human immunodeficiency virus
DDLYVGSDL

SEQ ID NO: 835 - protein, 9 aa, Human immunodeficiency virus
DLYVGSDLE

SEQ ID NO: 836 - protein, 9 aa, Human immunodeficiency virus
LYVGSDLEI

SEQ ID NO: 837 - protein, 9 aa, Human immunodeficiency virus
KHQKEPPFL

SEQ ID NO: 838 - protein, 9 aa, Human immunodeficiency virus
HQKEPPFLW

SEQ ID NO: 839 - protein, 9 aa, Human immunodeficiency virus
QKEPPFLWM

SEQ ID NO: 840 - protein, 9 aa, Human immunodeficiency virus
KEPPFLWMG

SEQ ID NO: 841 - protein, 9 aa, Human immunodeficiency virus
EPPFLWMGY

SEQ ID NO: 842 - protein, 9 aa, Human immunodeficiency virus
PPFLWMGYE

SEQ ID NO: 843 - protein, 9 aa, Human immunodeficiency virus
PFLWMGYEL

SEQ ID NO: 844 - protein, 9 aa, Human immunodeficiency virus
FLWMGYELH

SEQ ID NO: 845 - protein, 9 aa, Human immunodeficiency virus
LWMGYELHP

SEQ ID NO: 846 - protein, 9 aa, Human immunodeficiency virus
WMGYELHPD

SEQ ID NO: 847 - protein, 9 aa, Human immunodeficiency virus
MGYELHPDK

SEQ ID NO: 848 - protein, 9 aa, Human immunodeficiency virus
GYELHPDKW

SEQ ID NO: 849 - protein, 9 aa, Human immunodeficiency virus
YELHPDKWT

SEQ ID NO: 850 - protein, 9 aa, Human immunodeficiency virus
ELHPDKWTV

SEQ ID NO: 851 - protein, 9 aa, Human immunodeficiency virus
LHPDKWTVQ

SEQ ID NO: 852 - protein, 9 aa, Human immunodeficiency virus
HPDKWTVQP

SEQ ID NO: 853 - protein, 9 aa, Human immunodeficiency virus
PDKWTVQPI

SEQ ID NO: 854 - protein, 9 aa, Human immunodeficiency virus
LPEKDSWTV

SEQ ID NO: 855 - protein, 9 aa, Human immunodeficiency virus
PEKDSWTVN

SEQ ID NO: 856 - protein, 9 aa, Human immunodeficiency virus
EKDSWTVND

SEQ ID NO: 857 - protein, 9 aa, Human immunodeficiency virus
KDSWTVNDI

SEQ ID NO: 858 - protein, 9 aa, Human immunodeficiency virus
DSWTVNDIQ

SEQ ID NO: 859 - protein, 9 aa, Human immunodeficiency virus
SWTVNDIQK

SEQ ID NO: 860 - protein, 9 aa, Human immunodeficiency virus
WTVNDIQKL

SEQ ID NO: 861 - protein, 9 aa, Human immunodeficiency virus
TVNDIQKLV

SEQ ID NO: 862 - protein, 9 aa, Human immunodeficiency virus
VNDIQKLVG

SEQ ID NO: 863 - protein, 9 aa, Human immunodeficiency virus
NDIQKLVGK

SEQ ID NO: 864 - protein, 9 aa, Human immunodeficiency virus
DIQKLVGKL

SEQ ID NO: 865 - protein, 9 aa, Human immunodeficiency virus
IQKLVGKLN

SEQ ID NO: 866 - protein, 9 aa, Human immunodeficiency virus
QKLVGKLNW

SEQ ID NO: 867 - protein, 9 aa, Human immunodeficiency virus
KLVGKLNWA

SEQ ID NO: 868 - protein, 9 aa, Human immunodeficiency virus
LVGKLNWAS

SEQ ID NO: 869 - protein, 9 aa, Human immunodeficiency virus
VGKLNWASQ

SEQ ID NO: 870 - protein, 9 aa, Human immunodeficiency virus
GKLNWASQI

SEQ ID NO: 871 - protein, 9 aa, Human immunodeficiency virus
KLNWASQIY

SEQ ID NO: 872 - protein, 9 aa, Human immunodeficiency virus
EAELELAEN

SEQ ID NO: 873 - protein, 9 aa, Human immunodeficiency virus
AELELAENR

SEQ ID NO: 874 - protein, 9 aa, Human immunodeficiency virus
ELELAENRE

SEQ ID NO: 875 - protein, 9 aa, Human immunodeficiency virus
LELAENREI

SEQ ID NO: 876 - protein, 9 aa, Human immunodeficiency virus
ELAENREIL

SEQ ID NO: 877 - protein, 9 aa, Human immunodeficiency virus
LAENREILK

SEQ ID NO: 878 - protein, 9 aa, Human immunodeficiency virus
AENREILKE

SEQ ID NO: 879 - protein, 9 aa, Human immunodeficiency virus
ENREILKEP

SEQ ID NO: 880 - protein, 9 aa, Human immunodeficiency virus
NREILKEPV

SEQ ID NO: 881 - protein, 9 aa, Human immunodeficiency virus
REILKEPVH

SEQ ID NO: 882 - protein, 9 aa, Human immunodeficiency virus
EILKEPVHG

SEQ ID NO: 883 - protein, 9 aa, Human immunodeficiency virus
FLDGIDKAQ

SEQ ID NO: 884 - protein, 9 aa, Human immunodeficiency virus
EIVASCDKC

SEQ ID NO: 885 - protein, 9 aa, Human immunodeficiency virus
CDKCQLKGE

SEQ ID NO: 886 - protein, 9 aa, Human immunodeficiency virus
DKCQLKGEA

SEQ ID NO: 887 - protein, 9 aa, Human immunodeficiency virus
HGQVDCSPG

SEQ ID NO: 888 - protein, 9 aa, Human immunodeficiency virus
GQVDCSPGI

SEQ ID NO: 889 - protein, 9 aa, Human immunodeficiency virus
QVDCSPGIW

SEQ ID NO: 890 - protein, 9 aa, Human immunodeficiency virus
VDCSPGIWQ

SEQ ID NO: 891 - protein, 9 aa, Human immunodeficiency virus
DCSPGIWQL

SEQ ID NO: 892 - protein, 9 aa, Human immunodeficiency virus
CSPGIWQLD

SEQ ID NO: 893 - protein, 9 aa, Human immunodeficiency virus
SPGIWQLDC

SEQ ID NO: 894 - protein, 9 aa, Human immunodeficiency virus
PGIWQLDCT

SEQ ID NO: 895 - protein, 9 aa, Human immunodeficiency virus
GIWQLDCTH

SEQ ID NO: 896 - protein, 9 aa, Human immunodeficiency virus
IWQLDCTHL

SEQ ID NO: 897 - protein, 9 aa, Human immunodeficiency virus
WQLDCTHLE

SEQ ID NO: 898 - protein, 9 aa, Human immunodeficiency virus
QLDCTHLEG

SEQ ID NO: 899 - protein, 9 aa, Human immunodeficiency virus
LDCTHLEGK

SEQ ID NO: 900 - protein, 9 aa, Human immunodeficiency virus
ILVAVHVAS

SEQ ID NO: 901 - protein, 9 aa, Human immunodeficiency virus
LVAVHVASG

SEQ ID NO: 902 - protein, 9 aa, Human immunodeficiency virus
VAVHVASGY

SEQ ID NO: 903 - protein, 9 aa, Human immunodeficiency virus
AVHVASGYI

SEQ ID NO: 904 - protein, 9 aa, Human immunodeficiency virus
VHVASGYIE

SEQ ID NO: 905 - protein, 9 aa, Human immunodeficiency virus
HVASGYIEA

SEQ ID NO: 906 - protein, 9 aa, Human immunodeficiency virus
VASGYIEAE

SEQ ID NO: 907 - protein, 9 aa, Human immunodeficiency virus
ASGYIEAEV

SEQ ID NO: 908 - protein, 9 aa, Human immunodeficiency virus
SGYIEAEVI

SEQ ID NO: 909 - protein, 9 aa, Human immunodeficiency virus
GYIEAEVIP

SEQ ID NO: 910 - protein, 9 aa, Human immunodeficiency virus
YIEAEVIPA

SEQ ID NO: 911 - protein, 9 aa, Human immunodeficiency virus
IEAEVIPAE

SEQ ID NO: 912 - protein, 9 aa, Human immunodeficiency virus
EAEVIPAET

SEQ ID NO: 913 - protein, 9 aa, Human immunodeficiency virus
AEVIPAETG

SEQ ID NO: 914 - protein, 9 aa, Human immunodeficiency virus
EVIPAETGQ

SEQ ID NO: 915 - protein, 9 aa, Human immunodeficiency virus
VIPAETGQE

SEQ ID NO: 916 - protein, 9 aa, Human immunodeficiency virus
IPAETGQET

SEQ ID NO: 917 - protein, 9 aa, Human immunodeficiency virus
PAETGQETA

SEQ ID NO: 918 - protein, 9 aa, Human immunodeficiency virus
AETGQETAY

SEQ ID NO: 919 - protein, 9 aa, Human immunodeficiency virus
ETGQETAYF

SEQ ID NO: 920 - protein, 9 aa, Human immunodeficiency virus
LKLAGRWPV

SEQ ID NO: 921 - protein, 9 aa, Human immunodeficiency virus
KLAGRWPVK

SEQ ID NO: 922 - protein, 9 aa, Human immunodeficiency virus
VKAACWWAG

SEQ ID NO: 923 - protein, 9 aa, Human immunodeficiency virus
KAACWWAGI

SEQ ID NO: 924 - protein, 9 aa, Human immunodeficiency virus
ACWWAGIKQ

SEQ ID NO: 925 - protein, 9 aa, Human immunodeficiency virus
CWWAGIKQE

SEQ ID NO: 926 - protein, 9 aa, Human immunodeficiency virus
WWAGIKQEF

SEQ ID NO: 927 - protein, 9 aa, Human immunodeficiency virus
WAGIKQEFG

SEQ ID NO: 928 - protein, 9 aa, Human immunodeficiency virus
AGIKQEFGI

SEQ ID NO: 929 - protein, 9 aa, Human immunodeficiency virus
GIKQEFGIP

SEQ ID NO: 930 - protein, 9 aa, Human immunodeficiency virus
IKQEFGIPY

SEQ ID NO: 931 - protein, 9 aa, Human immunodeficiency virus
KQEFGIPYN

SEQ ID NO: 932 - protein, 9 aa, Human immunodeficiency virus
QEFGIPYNP

SEQ ID NO: 933 - protein, 9 aa, Human immunodeficiency virus
EFGIPYNPQ

SEQ ID NO: 934 - protein, 9 aa, Human immunodeficiency virus
FGIPYNPQS

SEQ ID NO: 935 - protein, 9 aa, Human immunodeficiency virus
GIPYNPQSQ

SEQ ID NO: 936 - protein, 9 aa, Human immunodeficiency virus
IPYNPQSQG

SEQ ID NO: 937 - protein, 9 aa, Human immunodeficiency virus
PYNPQSQGV

SEQ ID NO: 938 - protein, 9 aa, Human immunodeficiency virus
YNPQSQGVV

SEQ ID NO: 939 - protein, 9 aa, Human immunodeficiency virus
NPQSQGVVE

SEQ ID NO: 940 - protein, 9 aa, Human immunodeficiency virus
PQSQGVVES

SEQ ID NO: 941 - protein, 9 aa, Human immunodeficiency virus
QSQGVVESM

SEQ ID NO: 942 - protein, 9 aa, Human immunodeficiency virus
SQGVVESMN

SEQ ID NO: 943 - protein, 9 aa, Human immunodeficiency virus
ELKKIIGQV

SEQ ID NO: 944 - protein, 9 aa, Human immunodeficiency virus
LKKIIGQVR

SEQ ID NO: 945 - protein, 9 aa, Human immunodeficiency virus
IIGQVRDQA

SEQ ID NO: 946 - protein, 9 aa, Human immunodeficiency virus
IGQVRDQAE

SEQ ID NO: 947 - protein, 9 aa, Human immunodeficiency virus
GQVRDQAEH

SEQ ID NO: 948 - protein, 9 aa, Human immunodeficiency virus
QVRDQAEHL

SEQ ID NO: 949 - protein, 9 aa, Human immunodeficiency virus
VRDQAEHLK

SEQ ID NO: 950 - protein, 9 aa, Human immunodeficiency virus
RDQAEHLKT

SEQ ID NO: 951 - protein, 9 aa, Human immunodeficiency virus
DQAEHLKTA

SEQ ID NO: 952 - protein, 9 aa, Human immunodeficiency virus
QAEHLKTAV

SEQ ID NO: 953 - protein, 9 aa, Human immunodeficiency virus
AEHLKTAVQ

SEQ ID NO: 954 - protein, 9 aa, Human immunodeficiency virus
EHLKTAVQM

SEQ ID NO: 955 - protein, 9 aa, Human immunodeficiency virus
HLKTAVQMA

SEQ ID NO: 956 - protein, 9 aa, Human immunodeficiency virus
LKTAVQMAV

SEQ ID NO: 957 - protein, 9 aa, Human immunodeficiency virus
KTAVQMAVF

SEQ ID NO: 958 - protein, 9 aa, Human immunodeficiency virus
TAVQMAVFI

SEQ ID NO: 959 - protein, 9 aa, Human immunodeficiency virus
AVQMAVFIH

SEQ ID NO: 960 - protein, 9 aa, Human immunodeficiency virus
VQMAVFIHN

SEQ ID NO: 961 - protein, 9 aa, Human immunodeficiency virus
QMAVFIHNF

SEQ ID NO: 962 - protein, 9 aa, Human immunodeficiency virus
MAVFIHNFK

SEQ ID NO: 963 - protein, 9 aa, Human immunodeficiency virus
AVFIHNFKR

SEQ ID NO: 964 - protein, 9 aa, Human immunodeficiency virus
VFIHNFKRK

SEQ ID NO: 965 - protein, 9 aa, Human immunodeficiency virus
FIHNFKRKG

SEQ ID NO: 966 - protein, 9 aa, Human immunodeficiency virus
IHNFKRKGG

SEQ ID NO: 967 - protein, 9 aa, Human immunodeficiency virus
HNFKRKGGI

SEQ ID NO: 968 - protein, 9 aa, Human immunodeficiency virus
NFKRKGGIG

SEQ ID NO: 969 - protein, 9 aa, Human immunodeficiency virus
FKRKGGIGG

SEQ ID NO: 970 - protein, 9 aa, Human immunodeficiency virus
KRKGGIGGY

SEQ ID NO: 971 - protein, 9 aa, Human immunodeficiency virus
RKGGIGGYS

SEQ ID NO: 972 - protein, 9 aa, Human immunodeficiency virus
KGGIGGYSA

SEQ ID NO: 973 - protein, 9 aa, Human immunodeficiency virus
GGIGGYSAG

SEQ ID NO: 974 - protein, 9 aa, Human immunodeficiency virus
GIGGYSAGE

SEQ ID NO: 975 - protein, 9 aa, Human immunodeficiency virus
IGGYSAGER

SEQ ID NO: 976 - protein, 9 aa, Human immunodeficiency virus
GGYSAGERI

SEQ ID NO: 977 - protein, 9 aa, Human immunodeficiency virus
KIQNFRVYY

SEQ ID NO: 978 - protein, 9 aa, Human immunodeficiency virus
IQNFRVYYR

SEQ ID NO: 979 - protein, 9 aa, Human immunodeficiency virus
QNFRVYYRD

SEQ ID NO: 980 - protein, 9 aa, Human immunodeficiency virus
FRVYYRDSR

SEQ ID NO: 981 - protein, 9 aa, Human immunodeficiency virus
PLWKGPAKL

SEQ ID NO: 982 - protein, 9 aa, Human immunodeficiency virus
LWKGPAKLL

SEQ ID NO: 983 - protein, 9 aa, Human immunodeficiency virus
WKGPAKLLW

SEQ ID NO: 984 - protein, 9 aa, Human immunodeficiency virus
KGPAKLLWK

SEQ ID NO: 985 - protein, 9 aa, Human immunodeficiency virus
GPAKLLWKG

SEQ ID NO: 986 - protein, 9 aa, Human immunodeficiency virus
PAKLLWKGE

SEQ ID NO: 987 - protein, 9 aa, Human immunodeficiency virus
AKLLWKGEG

SEQ ID NO: 988 - protein, 9 aa, Human immunodeficiency virus
KLLWKGEGA

SEQ ID NO: 989 - protein, 9 aa, Human immunodeficiency virus
LLWKGEGAV

SEQ ID NO: 990 - protein, 9 aa, Human immunodeficiency virus
LWKGEGAVV

SEQ ID NO: 991 - protein, 9 aa, Human immunodeficiency virus
WKGEGAVVI

SEQ ID NO: 992 - protein, 9 aa, Human immunodeficiency virus
KGEGAVVIQ

SEQ ID NO: 993 - protein, 9 aa, Human immunodeficiency virus
GEGAVVIQD

SEQ ID NO: 994 - protein, 9 aa, Human immunodeficiency virus
EGAVVIQDN

SEQ ID NO: 995 - protein, 9 aa, Human immunodeficiency virus
GAVVIQDNS

SEQ ID NO: 996 - protein, 9 aa, Human immunodeficiency virus
KIIRDYGKQ

SEQ ID NO: 997 - protein, 9 aa, Human immunodeficiency virus
IIRDYGKQM

SEQ ID NO: 998 - protein, 9 aa, Human immunodeficiency virus
IRDYGKQMA

SEQ ID NO: 999 - protein, 9 aa, Human immunodeficiency virus
RDYGKQMAG

SEQ ID NO: 1000 - protein, 9 aa, Human immunodeficiency virus
DYGKQMAGD

SEQ ID NO: 1001 - protein, 9 aa, Human immunodeficiency virus
YGKQMAGDD

SEQ ID NO: 1002 - protein, 9 aa, Human immunodeficiency virus
GKQMAGDDC

SEQ ID NO: 1003 - protein, 9 aa, Human immunodeficiency virus
KQMAGDDCV

SEQ ID NO: 1004 - protein, 9 aa, Human immunodeficiency virus
QMAGDDCVA

SEQ ID NO: 1005 - protein, 9 aa, Human immunodeficiency virus
MENRWQVMI

SEQ ID NO: 1006 - protein, 9 aa, Human immunodeficiency virus
ENRWQVMIV

SEQ ID NO: 1007 - protein, 9 aa, Human immunodeficiency virus
NRWQVMIVW

SEQ ID NO: 1008 - protein, 9 aa, Human immunodeficiency virus
RWQVMIVWQ

SEQ ID NO: 1009 - protein, 9 aa, Human immunodeficiency virus
WQVMIVWQV

SEQ ID NO: 1010 - protein, 9 aa, Human immunodeficiency virus
QVMIVWQVD

SEQ ID NO: 1011 - protein, 9 aa, Human immunodeficiency virus
VMIVWQVDR

SEQ ID NO: 1012 - protein, 9 aa, Human immunodeficiency virus
MIVWQVDRM

SEQ ID NO: 1013 - protein, 9 aa, Human immunodeficiency virus
IVWQVDRMR

SEQ ID NO: 1014 - protein, 9 aa, Human immunodeficiency virus
VWQVDRMRI

SEQ ID NO: 1015 - protein, 9 aa, Human immunodeficiency virus
SSEVHIPLG

SEQ ID NO: 1016 - protein, 9 aa, Human immunodeficiency virus
TYWGLHTGE

SEQ ID NO: 1017 - protein, 9 aa, Human immunodeficiency virus
YWGLHTGER

SEQ ID NO: 1018 - protein, 9 aa, Human immunodeficiency virus
WHLGQGVSI

SEQ ID NO: 1019 - protein, 9 aa, Human immunodeficiency virus
HLGQGVSIE

SEQ ID NO: 1020 - protein, 9 aa, Human immunodeficiency virus
LGQGVSIEW

SEQ ID NO: 1021 - protein, 9 aa, Human immunodeficiency virus
GQGVSIEWR

SEQ ID NO: 1022 - protein, 9 aa, Human immunodeficiency virus
GHNKVGSLQ

SEQ ID NO: 1023 - protein, 9 aa, Human immunodeficiency virus
HNKVGSLQY

SEQ ID NO: 1024 - protein, 9 aa, Human immunodeficiency virus
NKVGSLQYL

SEQ ID NO: 1025 - protein, 9 aa, Human immunodeficiency virus
KVGSLQYLA

SEQ ID NO: 1026 - protein, 9 aa, Human immunodeficiency virus
VGSLQYLAL

SEQ ID NO: 1027 - protein, 9 aa, Human immunodeficiency virus
KLTEDRWNK

SEQ ID NO: 1028 - protein, 9 aa, Human immunodeficiency virus
LTEDRWNKP

SEQ ID NO: 1029 - protein, 9 aa, Human immunodeficiency virus
TEDRWNKPQ

SEQ ID NO: 1030 - protein, 9 aa, Human immunodeficiency virus
MEQAPEDQG

SEQ ID NO: 1031 - protein, 9 aa, Human immunodeficiency virus
EQAPEDQGP

SEQ ID NO: 1032 - protein, 9 aa, Human immunodeficiency virus
QAPEDQGPQ

SEQ ID NO: 1033 - protein, 9 aa, Human immunodeficiency virus
APEDQGPQR

SEQ ID NO: 1034 - protein, 9 aa, Human immunodeficiency virus
PEDQGPQRE

SEQ ID NO: 1035 - protein, 9 aa, Human immunodeficiency virus
EDQGPQREP

SEQ ID NO: 1036 - protein, 9 aa, Human immunodeficiency virus
WTLELLEEL

SEQ ID NO: 1037 - protein, 9 aa, Human immunodeficiency virus
TLELLEELK

SEQ ID NO: 1038 - protein, 9 aa, Human immunodeficiency virus
LEPWKHPGS

SEQ ID NO: 1039 - protein, 9 aa, Human immunodeficiency virus
EPWKHPGSQ

SEQ ID NO: 1040 - protein, 9 aa, Human immunodeficiency virus
PWKHPGSQP

SEQ ID NO: 1041 - protein, 9 aa, Human immunodeficiency virus
LGISYGRKK

SEQ ID NO: 1042 - protein, 9 aa, Human immunodeficiency virus
GISYGRKKR

SEQ ID NO: 1043 - protein, 9 aa, Human immunodeficiency virus
ISYGRKKRR

SEQ ID NO: 1044 - protein, 9 aa, Human immunodeficiency virus
SYGRKKRRQ

SEQ ID NO: 1045 - protein, 9 aa, Human immunodeficiency virus
YGRKKRRQR

SEQ ID NO: 1046 - protein, 9 aa, Human immunodeficiency virus
GRKKRRQRR

SEQ ID NO: 1047 - protein, 9 aa, Human immunodeficiency virus
RKKRRQRRR

SEQ ID NO: 1048 - protein, 9 aa, Human immunodeficiency virus
EGTRQARRN

SEQ ID NO: 1049 - protein, 9 aa, Human immunodeficiency virus
GTRQARRNR

SEQ ID NO: 1050 - protein, 9 aa, Human immunodeficiency virus
TRQARRNRR

SEQ ID NO: 1051 - protein, 9 aa, Human immunodeficiency virus
RQARRNRRR

SEQ ID NO: 1052 - protein, 9 aa, Human immunodeficiency virus
QARRNRRRR

SEQ ID NO: 1053 - protein, 9 aa, Human immunodeficiency virus
ARRNRRRRW

SEQ ID NO: 1054 - protein, 9 aa, Human immunodeficiency virus
RRNRRRRWR

SEQ ID NO: 1055 - protein, 9 aa, Human immunodeficiency virus
ERAEDSGNE

SEQ ID NO: 1056 - protein, 9 aa, Human immunodeficiency virus
RAEDSGNES

SEQ ID NO: 1057 - protein, 9 aa, Human immunodeficiency virus
LWVTVYYGV

SEQ ID NO: 1058 - protein, 9 aa, Human immunodeficiency virus
WVTVYYGVP

SEQ ID NO: 1059 - protein, 9 aa, Human immunodeficiency virus
VTVYYGVPV

SEQ ID NO: 1060 - protein, 9 aa, Human immunodeficiency virus
TVYYGVPVW

SEQ ID NO: 1061 - protein, 9 aa, Human immunodeficiency virus
VYYGVPVWK

SEQ ID NO: 1062 - protein, 9 aa, Human immunodeficiency virus
YYGVPVWKE

SEQ ID NO: 1063 - protein, 9 aa, Human immunodeficiency virus
YGVPVWKEA

SEQ ID NO: 1064 - protein, 9 aa, Human immunodeficiency virus
GVPVWKEAT

SEQ ID NO: 1065 - protein, 9 aa, Human immunodeficiency virus
VPVWKEATT

SEQ ID NO: 1066 - protein, 9 aa, Human immunodeficiency virus
PVWKEATTT

SEQ ID NO: 1067 - protein, 9 aa, Human immunodeficiency virus
VWKEATTTL

SEQ ID NO: 1068 - protein, 9 aa, Human immunodeficiency virus
WKEATTTLF

SEQ ID NO: 1069 - protein, 9 aa, Human immunodeficiency virus
KEATTTLFC

SEQ ID NO: 1070 - protein, 9 aa, Human immunodeficiency virus
EATTTLFCA

SEQ ID NO: 1071 - protein, 9 aa, Human immunodeficiency virus
ATTTLFCAS

SEQ ID NO: 1072 - protein, 9 aa, Human immunodeficiency virus
TTTLFCASD

SEQ ID NO: 1073 - protein, 9 aa, Human immunodeficiency virus
TTLFCASDA

SEQ ID NO: 1074 - protein, 9 aa, Human immunodeficiency virus
TLFCASDAK

SEQ ID NO: 1075 - protein, 9 aa, Human immunodeficiency virus
HNVWATHAC

SEQ ID NO: 1076 - protein, 9 aa, Human immunodeficiency virus
NVWATHACV

SEQ ID NO: 1077 - protein, 9 aa, Human immunodeficiency virus
VWATHACVP

SEQ ID NO: 1078 - protein, 9 aa, Human immunodeficiency virus
WATHACVPT

SEQ ID NO: 1079 - protein, 9 aa, Human immunodeficiency virus
SLKPCVKLT

SEQ ID NO: 1080 - protein, 9 aa, Human immunodeficiency virus
LKPCVKLTP

SEQ ID NO: 1081 - protein, 9 aa, Human immunodeficiency virus
KPCVKLTPL

SEQ ID NO: 1082 - protein, 9 aa, Human immunodeficiency virus
PCVKLTPLC

SEQ ID NO: 1083 - protein, 9 aa, Human immunodeficiency virus
CVKLTPLCV

SEQ ID NO: 1084 - protein, 9 aa, Human immunodeficiency virus
VKLTPLCVT

SEQ ID NO: 1085 - protein, 9 aa, Human immunodeficiency virus
KLTPLCVTL

SEQ ID NO: 1086 - protein, 9 aa, Human immunodeficiency virus
NVSTVQCTH

SEQ ID NO: 1087 - protein, 9 aa, Human immunodeficiency virus
VSTVQCTHG

SEQ ID NO: 1088 - protein, 9 aa, Human immunodeficiency virus
STVQCTHGI

SEQ ID NO: 1089 - protein, 9 aa, Human immunodeficiency virus
PVVSTQLLL

SEQ ID NO: 1090 - protein, 9 aa, Human immunodeficiency virus
VVSTQLLLN

SEQ ID NO: 1091 - protein, 9 aa, Human immunodeficiency virus
VSTQLLLNG

SEQ ID NO: 1092 - protein, 9 aa, Human immunodeficiency virus
STQLLLNGS

SEQ ID NO: 1093 - protein, 9 aa, Human immunodeficiency virus
TQLLLNGSL

SEQ ID NO: 1094 - protein, 9 aa, Human immunodeficiency virus
QLLLNGSLA

SEQ ID NO: 1095 - protein, 9 aa, Human immunodeficiency virus
LLLNGSLAE

SEQ ID NO: 1096 - protein, 9 aa, Human immunodeficiency virus
VGKAMYAPP

SEQ ID NO: 1097 - protein, 9 aa, Human immunodeficiency virus
GKAMYAPPI

SEQ ID NO: 1098 - protein, 9 aa, Human immunodeficiency virus
DNWRSELYK

SEQ ID NO: 1099 - protein, 9 aa, Human immunodeficiency virus
NWRSELYKY

SEQ ID NO: 1100 - protein, 9 aa, Human immunodeficiency virus
WRSELYKYK

SEQ ID NO: 1101 - protein, 9 aa, Human immunodeficiency virus
RSELYKYKV

SEQ ID NO: 1102 - protein, 9 aa, Human immunodeficiency virus
SELYKYKVV

SEQ ID NO: 1103 - protein, 9 aa, Human immunodeficiency virus
AKRRVVQRE

SEQ ID NO: 1104 - protein, 9 aa, Human immunodeficiency virus
FLGFLGAAG

SEQ ID NO: 1105 - protein, 9 aa, Human immunodeficiency virus
LGFLGAAGS

SEQ ID NO: 1106 - protein, 9 aa, Human immunodeficiency virus
GFLGAAGST

SEQ ID NO: 1107 - protein, 9 aa, Human immunodeficiency virus
FLGAAGSTM

SEQ ID NO: 1108 - protein, 9 aa, Human immunodeficiency virus
LGAAGSTMG

SEQ ID NO: 1109 - protein, 9 aa, Human immunodeficiency virus
GAAGSTMGA

SEQ ID NO: 1110 - protein, 9 aa, Human immunodeficiency virus
AAGSTMGAA

SEQ ID NO: 1111 - protein, 9 aa, Human immunodeficiency virus
AGSTMGAAS

SEQ ID NO: 1112 - protein, 9 aa, Human immunodeficiency virus
LLSGIVQQQ

SEQ ID NO: 1113 - protein, 9 aa, Human immunodeficiency virus
LQLTVWGIK

SEQ ID NO: 1114 - protein, 9 aa, Human immunodeficiency virus
QLTVWGIKQ

SEQ ID NO: 1115 - protein, 9 aa, Human immunodeficiency virus
LTVWGIKQL

SEQ ID NO: 1116 - protein, 9 aa, Human immunodeficiency virus
TVWGIKQLQ

SEQ ID NO: 1117 - protein, 9 aa, Human immunodeficiency virus
VWGIKQLQA

SEQ ID NO: 1118 - protein, 9 aa, Human immunodeficiency virus
WGIKQLQAR

SEQ ID NO: 1119 - protein, 9 aa, Human immunodeficiency virus
GIKQLQARV

SEQ ID NO: 1120 - protein, 9 aa, Human immunodeficiency virus
IKQLQARVL

SEQ ID NO: 1121 - protein, 9 aa, Human immunodeficiency virus
KQLQARVLA

SEQ ID NO: 1122 - protein, 9 aa, Human immunodeficiency virus
DQQLLGIWG

SEQ ID NO: 1123 - protein, 9 aa, Human immunodeficiency virus
QQLLGIWGC

SEQ ID NO: 1124 - protein, 9 aa, Human immunodeficiency virus
QLLGIWGCS

SEQ ID NO: 1125 - protein, 9 aa, Human immunodeficiency virus
LLGIWGCSG

SEQ ID NO: 1126 - protein, 9 aa, Human immunodeficiency virus
LGIWGCSGK

SEQ ID NO: 1127 - protein, 9 aa, Human immunodeficiency virus
GIWGCSGKL

SEQ ID NO: 1128 - protein, 9 aa, Human immunodeficiency virus
IWGCSGKLI

SEQ ID NO: 1129 - protein, 9 aa, Human immunodeficiency virus
WGCSGKLIC

SEQ ID NO: 1130 - protein, 9 aa, Human immunodeficiency virus
GCSGKLICT

SEQ ID NO: 1131 - protein, 9 aa, Human immunodeficiency virus
CSGKLICTT

SEQ ID NO: 1132 - protein, 9 aa, Human immunodeficiency virus
WLWYIKIFI

SEQ ID NO: 1133 - protein, 9 aa, Human immunodeficiency virus
AIAVAEGTD

SEQ ID NO: 1134 - protein, 9 aa, Human immunodeficiency virus
IAVAEGTDR

SEQ ID NO: 1135 - protein, 9 aa, Human immunodeficiency virus
PQVPLRPMT

SEQ ID NO: 1136 - protein, 9 aa, Human immunodeficiency virus
FPDWQNYTP

SEQ ID NO: 1137 - protein, 9 aa, Human immunodeficiency virus
PDWQNYTPG

SEQ ID NO: 1138 - protein, 9 aa, Human immunodeficiency virus
DWQNYTPGP

SEQ ID NO: 1139 - protein, 9 aa, Human immunodeficiency virus
WQNYTPGPG

SEQ ID NO: 1140 - protein, 9 aa, Human immunodeficiency virus
FGWCFKLVP

SEQ ID NO: 1141 - protein, 15 aa, Human immunodeficiency virus
EKIRLRPGGKKKYKL

SEQ ID NO: 1142 - protein, 15 aa, Human immunodeficiency virus
YKLKHIVWASRELER

SEQ ID NO: 1143 - protein, 15 aa, Human immunodeficiency virus
HIVWASRELERFAVN

SEQ ID NO: 1144 - protein, 11 aa, Human immunodeficiency virus
LVWASRELERF

SEQ ID NO: 1145 - protein, 9 aa, Human immunodeficiency virus
WASRELERF

SEQ ID NO: 1146 - protein, 15 aa, Human immunodeficiency virus
ASRELERFAVNPGLL

SEQ ID NO: 1147 - protein, 11 aa, Human immunodeficiency virus
SQNYPIVQNIQ

SEQ ID NO: 1148 - protein, 9 aa, Human immunodeficiency virus
SPRTLNAWV

SEQ ID NO: 1149 - protein, 10 aa, Human immunodeficiency virus
ISPRTLNAWV

SEQ ID NO: 1150 - protein, 9 aa, Human immunodeficiency virus
EEKAFSPEV

SEQ ID NO: 1151 - protein, 20 aa, Human immunodeficiency virus
TLNAWVKVVEEKAFSPEVIP

SEQ ID NO: 1152 - protein, 20 aa, Human immunodeficiency virus
EKAFSPEVIPMFSALSEGAT

SEQ ID NO: 1153 - protein, 15 aa, Human immunodeficiency virus
EKAFSPEVIPMFSAL

SEQ ID NO: 1154 - protein, 11 aa, Human immunodeficiency virus
KAFSPEVIPMF

SEQ ID NO: 1155 - protein, 11 aa, Human immunodeficiency virus
IEEKAFSPEVI

SEQ ID NO: 1156 - protein, 9 aa, Human immunodeficiency virus
FSPEVIPMF

SEQ ID NO: 1157 - protein, 20 aa, Human immunodeficiency virus
SALSEGATPQDLNMMLNIVG

SEQ ID NO: 1158 - protein, 20 aa, Human immunodeficiency virus
MFSALSEGATPQDLNTMLNT

SEQ ID NO: 1159 - protein, 15 aa, Human immunodeficiency virus
IPMFSALSEGATPQD

SEQ ID NO: 1160 - protein, 15 aa, Human immunodeficiency virus
PQDLNTMLNTVGGHQ

SEQ ID NO: 1161 - protein, 10 aa, Human immunodeficiency virus
LSEGATPQDL

SEQ ID NO: 1162 - protein, 12 aa, Human immunodeficiency virus
ATPQDLNTMLNT

SEQ ID NO: 1163 - protein, 9 aa, Human immunodeficiency virus
TPQDLNTML

SEQ ID NO: 1164 - protein, 9 aa, Human immunodeficiency virus
PQDLNTMLN

SEQ ID NO: 1165 - protein, 9 aa, Human immunodeficiency virus
DLNTMLNTV

SEQ ID NO: 1166 - protein, 9 aa, Human immunodeficiency virus
GHQAAMQML

SEQ ID NO: 1167 - protein, 18 aa, Human immunodeficiency virus
ETINEEAAEWDRVHPVHA

SEQ ID NO: 1168 - protein, 20 aa, Human immunodeficiency virus
LKETINEEAAEWDRVHPVHA

SEQ ID NO: 1169 - protein, 18 aa, Human immunodeficiency virus
LKDTINEEAAEWDRLHPV

SEQ ID NO: 1170 - protein, 10 aa, Human immunodeficiency virus
DTINEEAAEW

SEQ ID NO: 1171 - protein, 10 aa, Human immunodeficiency virus
ETINEEAAEW

SEQ ID NO: 1172 - protein, 22 aa, Human immunodeficiency virus
ETINEEAAEWDRVHPVHAGPIA

SEQ ID NO: 1173 - protein, 11 aa, Human immunodeficiency virus
INEEAAEWDRV

SEQ ID NO: 1174 - protein, 14 aa, Human immunodeficiency virus
GSDIAGTTSTQEQI

SEQ ID NO: 1175 - protein, 15 aa, Human immunodeficiency virus
GSDIAGTTSTLQEQI

SEQ ID NO: 1176 - protein, 20 aa, Human immunodeficiency virus
PRGSDIAGTTSTLQEQIGWM

SEQ ID NO: 1177 - protein, 20 aa, Human immunodeficiency virus
GPIAPGQMREPRGSDIAGTT

SEQ ID NO: 1178 - protein, 11 aa, Human immunodeficiency virus
GQMREPRGSDI

SEQ ID NO: 1179 - protein, 15 aa, Human immunodeficiency virus
HAGPIAPGQMREPRG

SEQ ID NO: 1180 - protein, 20 aa, Human immunodeficiency virus
TNNPPIPVGEIYKRWIILGL

SEQ ID NO: 1181 - protein, 9 aa, Human immunodeficiency virus
PPIPVGEIY

SEQ ID NO: 1182 - protein, 15 aa, Human immunodeficiency virus
GLNKIVRMYSPTSIL

SEQ ID NO: 1183 - protein, 18 aa, Human immunodeficiency virus
WIILGLNKIVRMYSPTSI

SEQ ID NO: 1184 - protein, 11 aa, Human immunodeficiency virus
ILGLNKIVRMY

SEQ ID NO: 1185 - protein, 15 aa, Human immunodeficiency virus
GLNKIVRMYSPTSIL

SEQ ID NO: 1186 - protein, 15 aa, Human immunodeficiency virus
WIILGLNKIVRMYSP

SEQ ID NO: 1187 - protein, 20 aa, Human immunodeficiency virus
IYKRWIILGLNKIVRMYSPT

SEQ ID NO: 1188 - protein, 20 aa, Human immunodeficiency virus
NKIVRMYSPTSILDIRQGPK

SEQ ID NO: 1189 - protein, 20 aa, Human immunodeficiency virus
KRWIILGLNKIVRMYSPTSI

SEQ ID NO: 1190 - protein, 9 aa, Human immunodeficiency virus
GLNKIVRMY

SEQ ID NO: 1191 - protein, 15 aa, Human immunodeficiency virus
NKIVRMYSPVSILDI

SEQ ID NO: 1192 - protein, 15 aa, Human immunodeficiency virus
SILDIKQGPKEPFRD

SEQ ID NO: 1193 - protein, 15 aa, Human immunodeficiency virus
RQGPKEPFRDYVDRF

SEQ ID NO: 1194 - protein, 9 aa, Human immunodeficiency virus
PKEPFRDYV

SEQ ID NO: 1195 - protein, 20 aa, Human immunodeficiency virus
EPFRDYVDRFYKTLRAEQAS

SEQ ID NO: 1196 - protein, 10 aa, Human immunodeficiency virus
EPFRDYVDRF

SEQ ID NO: 1197 - protein, 10 aa, Human immunodeficiency virus
FRDYVDRFYK

SEQ ID NO: 1198 - protein, 15 aa, Human immunodeficiency virus
FRDYVDRFYKTLRAE

SEQ ID NO: 1199 - protein, 11 aa, Human immunodeficiency virus
RDYVDRFYKTL

SEQ ID NO: 1200 - protein, 11 aa, Human immunodeficiency virus
DYVDRFYKTLR

SEQ ID NO: 1201 - protein, 9 aa, Human immunodeficiency virus
DYVDRFYKT

SEQ ID NO: 1202 - protein, 18 aa, Human immunodeficiency virus
YVDRFYKTLRAEQASQEV

SEQ ID NO: 1203 - protein, 9 aa, Human immunodeficiency virus
YVDRFYKTL

SEQ ID NO: 1204 - protein, 15 aa, Human immunodeficiency virus
VDRFYKTLRAEQASQ

SEQ ID NO: 1205 - protein, 9 aa, Human immunodeficiency virus
DRFYKTLRA

SEQ ID NO: 1206 - protein, 11 aa, Human immunodeficiency virus
DRFYKTLRAEQ

SEQ ID NO: 1207 - protein, 12 aa, Human immunodeficiency virus
RFYKTLRAEQAS

SEQ ID NO: 1208 - protein, 13 aa, Human immunodeficiency virus
FYKTLRAEQASQE

SEQ ID NO: 1209 - protein, 12 aa, Human immunodeficiency virus
FYKTLRAEQASQ

SEQ ID NO: 1210 - protein, 9 aa, Human immunodeficiency virus
YKTLRAEQA

SEQ ID NO: 1211 - protein, 11 aa, Human immunodeficiency virus
YKTLRAEQASQ

SEQ ID NO: 1212 - protein, 9 aa, Human immunodeficiency virus
DCKTILKAL

SEQ ID NO: 1213 - protein, 18 aa, Human immunodeficiency virus
MTDTLLVQNANPDCKTIL

SEQ ID NO: 1214 - protein, 15 aa, Human immunodeficiency virus
VKNWMTETLLVQNAN

SEQ ID NO: 1215 - protein, 10 aa, Human immunodeficiency virus
VKNWMTETLL

SEQ ID NO: 1216 - protein, 13 aa, Human immunodeficiency virus
NANPDCKTILRAL

SEQ ID NO: 1217 - protein, 20 aa, Human immunodeficiency virus
GNFRNQRKIVKCFNCGKEGH

SEQ ID NO: 1218 - protein, 16 aa, Human immunodeficiency virus
RQANFLGKIWPSHKGR

SEQ ID NO: 1219 - protein, 10 aa, Human immunodeficiency virus
KMIGGIGGFI

SEQ ID NO: 1220 - protein, 10 aa, Human immunodeficiency virus
FPISPIETVP

SEQ ID NO: 1221 - protein, 9 aa, Human immunodeficiency virus
FPISPIETV

SEQ ID NO: 1222 - protein, 16 aa, Human immunodeficiency virus
PISPIETVPVKLKPGM

SEQ ID NO: 1223 - protein, 10 aa, Human immunodeficiency virus
SPIETVPVKL

SEQ ID NO: 1224 - protein, 18 aa, Human immunodeficiency virus
GMDGPKVKQWPLTEEKIK

SEQ ID NO: 1225 - protein, 18 aa, Human immunodeficiency virus
FWEVQLGIPHPAGLKKKK

SEQ ID NO: 1226 - protein, 9 aa, Human immunodeficiency virus
TVLDVGDAY

SEQ ID NO: 1227 - protein, 17 aa, Human immunodeficiency virus
KKKSVTVLDVGDAYFSV

SEQ ID NO: 1228 - protein, 9 aa, Human immunodeficiency virus
NNETPGIRY

SEQ ID NO: 1229 - protein, 11 aa, Human immunodeficiency virus
LPQGWKGSPAI

SEQ ID NO: 1230 - protein, 10 aa, Human immunodeficiency virus
LPQGWKGSPA

SEQ ID NO: 1231 - protein, 18 aa, Human immunodeficiency virus
DKKHQKEPPFLWMGYELH

SEQ ID NO: 1232 - protein, 18 aa, Human immunodeficiency virus
TVQPIQLPEKDSWTVNDI

SEQ ID NO: 1233 - protein, 17 aa, Human immunodeficiency virus
EKDSWTVNDIQKLVGKL

SEQ ID NO: 1234 - protein, 9 aa, Human immunodeficiency virus
KLVGKLNWA

SEQ ID NO: 1235 - protein, 9 aa, Human immunodeficiency virus
KLNWASQIY

SEQ ID NO: 1236 - protein, 18 aa, Human immunodeficiency virus
ELAENREILKEPVHGVYY

SEQ ID NO: 1237 - protein, 18 aa, Human immunodeficiency virus
PPIVAKEIVASCDKCQLK

SEQ ID NO: 1238 - protein, 11 aa, Human immunodeficiency virus
EIVASCDKCQL

SEQ ID NO: 1239 - protein, 15 aa, Human immunodeficiency virus
GKIILVAVHVASGYI

SEQ ID NO: 1240 - protein, 18 aa, Human immunodeficiency virus
PAETGQETAYFILKLAGR

SEQ ID NO: 1241 - protein, 9 aa, Human immunodeficiency virus
HVASGYIEA

SEQ ID NO: 1242 - protein, 10 aa, Human immunodeficiency virus
AETGQETAYY

SEQ ID NO: 1243 - protein, 11 aa, Human immunodeficiency virus
ILKLAGRWPVK

SEQ ID NO: 1244 - protein, 12 aa, Human immunodeficiency virus
IQQEFGIPYNPQ

SEQ ID NO: 1245 - protein, 11 aa, Human immunodeficiency virus
IPYNPQSQGVV

SEQ ID NO: 1246 - protein, 9 aa, Human immunodeficiency virus
QVRDQAEHL

SEQ ID NO: 1247 - protein, 10 aa, Human immunodeficiency virus
QMAVFIHNFK

SEQ ID NO: 1248 - protein, 10 aa, Human immunodeficiency virus
AVFIHNFKRK

SEQ ID NO: 1249 - protein, 10 aa, Human immunodeficiency virus
FKRKGGIGGY

SEQ ID NO: 1250 - protein, 18 aa, Human immunodeficiency virus
RKGGIGGYSAGERIVDII

SEQ ID NO: 1251 - protein, 9 aa, Human immunodeficiency virus
KIQNFRVYY

SEQ ID NO: 1252 - protein, 10 aa, Human immunodeficiency virus
TKIQNFRVYY

SEQ ID NO: 1253 - protein, 10 aa, Human immunodeficiency virus
KIQNFRVYYR

SEQ ID NO: 1254 - protein, 18 aa, Human immunodeficiency virus
LWKGEGAVVIQDNSDIKV

SEQ ID NO: 1255 - protein, 18 aa, Human immunodeficiency virus
GPQREPYNEWTLELLEEL

SEQ ID NO: 1256 - protein, 15 aa, Human immunodeficiency virus
KALGISYGRKKRRQR

SEQ ID NO: 1257 - protein, 19 aa, Human immunodeficiency virus
ERAEDSGNESEGDTEELSA

SEQ ID NO: 1258 - protein, 10 aa, Human immunodeficiency virus
KLWVTVYYGV

SEQ ID NO: 1259 - protein, 9 aa, Human immunodeficiency virus
LWVTVYYGV

SEQ ID NO: 1260 - protein, 22 aa, Human immunodeficiency virus
LWVTVYYGVPVWKEATTTLFCA

SEQ ID NO: 1261 - protein, 10 aa, Human immunodeficiency virus
TVYYGVPVWK

SEQ ID NO: 1262 - protein, 9 aa, Human immunodeficiency virus
TVYYGVPVW

SEQ ID NO: 1263 - protein, 17 aa, Human immunodeficiency virus
TVYYGVPVWKEAKTTLF

SEQ ID NO: 1264 - protein, 11 aa, Human immunodeficiency virus
VTVYYGVPVWK

SEQ ID NO: 1265 - protein, 10 aa, Human immunodeficiency virus
TTLFCASDAK

SEQ ID NO: 1266 - protein, 9 aa, Human immunodeficiency virus
KLTPLCVTL

SEQ ID NO: 1267 - protein, 9 aa, Human immunodeficiency virus
WLWYIKIFI

SEQ ID NO: 1268 - protein, 11 aa, Human immunodeficiency virus
TPQVPLRPMTY

SEQ ID NO: 1269 - protein, 15 aa, Human immunodeficiency virus
FPVRPQVPLRPMTYK

SEQ ID NO: 1270 - protein, 15 aa, Human immunodeficiency virus
PVRPQVPLRPMTYKA

SEQ ID NO: 1271 - protein, 9 aa, Human immunodeficiency virus
FPDWQNYTP

SEQ ID NO: 1272 - protein, 16 aa, Human immunodeficiency virus
GIRYPLTFGWCFKLVP

SEQ ID NO: 1273 - protein, 380 aa, Homo sapiens
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENS

SEQ ID NO: 1274 - protein, 36 aa, Homo sapiens
TLIPIAVGGALAGLVLIVLIAYLVGRKRSHAGYQTI

SEQ ID NO: 1275 - DNA, 1140 bp, Homo sapiens
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagc

SEQ ID NO: 1276 - DNA, 117 bp, Homo sapiens
acgctgatccccatcgctgtgggtggtgccctggcggggctggtcctcatcgtcctcatcgcctacctcgtcggcaggaagaggagtcacgcaggctaccagactatctagggtacc

(19) United States
(12) Patent Application Publication
(10) Pub. No.: US 2013/0195904 A1
(43) Pub. Date: Aug. 1, 2013

(54) HUMAN IMMUNODEFICIENCY VIRUS (HIV-1) HIGHLY CONSERVED AND LOW VARIANT SEQUENCES AS TARGETS FOR VACCINE AND DIAGNOSTIC APPLICATIONS

(71) Applicants: J. Thomas August, Baltimore, MD (US); Gregory George Simon, Owings Mills, MD (US); Tin Wee Tan, Singapore (SG); Asif Mohammad Khan, Singapore (SG); Hu Yongli, Singapore (SG)

(72) Inventors: J. Thomas August, Baltimore, MD (US); Gregory George Simon, Owings Mills, MD (US); Tin Wee Tan, Singapore (SG); Asif Mohammad Khan, Singapore (SG); Hu Yongli, Singapore (SG)

(73) Assignees: NATIONAL UNIVERSITY OF SINGAPORE, Singapore (SG); THE JOHNS HOPKINS UNIVERSITY, Baltimore, MD (US)

(21) Appl. No.: 13/520,388

(22) PCT Filed: Jan. 4, 2011
(86) PCT No.: PCT/US11/20122

§ 371 Date: Oct. 17, 2012

Related U.S. Application Data
(60) Provisional application No. 61/292,068, filed on Jan. 4, 2010.

(51) Int. Cl. A61K 39/21

(52) U.S. Cl. CPC A61K 39/21
USPC 424/186.1; 530/300; 536/23.72; 435/320.1; 435/325; 435/69.1; 435/5

(57) ABSTRACT

We identified regions of the HIV-1 proteome with high conservation, and low variant incidence. Such highly conserved sequences have direct relevance to the development of new-generation vaccines and diagnostic applications. The immune relevance of these sequences was assessed by their correlation to previously reported human T-cell epitopes and to recently identified human HIV-1 T-cell epitopes (identified using HLA transgenic mice). We identified (a) sequences specific to HIV-1 with no shared identity to other viruses and organisms, and (b) sequences that are specific to primate lentivirus group, with multiclade HIV-1 conservation.

This invention was made with funds from the U.S. government. Therefore the U.S. government retains certain rights in the invention according to the terms of grant no. R37 AI-041908.

### TECHNICAL FIELD OF THE INVENTION

This invention is related to the area of vaccines and immunity. In particular, it relates to vaccines for inducing immunity to Human Immunodeficiency Virus.

### BACKGROUND OF THE INVENTION

The rapid evolution of HIV-1 and resulting diversity in the viral proteomes is widely acknowledged as playing a major role in the failure of most infected individuals to control either acute or chronic HIV-1 infection (Abram et al., 2010; Goulder and Watkins, 2004; McMichael et al., 2010; Pereyra et al., 2010; Troyer et al., 2009). The sequence diversity of HIV-1 proteins is a combination of the frequency of mutations, about 1.4×10⁻⁵ per base pair (Abram et al., 2010), two to three recombination events per cycle of virus replication (Jetzt et al., 2000), and a high replication rate of about 10¹⁰ to 10¹² virions per day (Perelson et al., 1996). This leads to the rapid evolution of genetically distinct mutant viruses, which accumulate within the host as a complex mixture of viral quasispecies (Eigen, 1993). Survival of the individual variant viruses is determined by the relative host fitness and a complex association of mutations and immune escape through a multiplicity of mechanisms (Brumme et al., 2009; Brumme and Walker, 2009; Liang et al., 2008; Wang et al., 2009). This process is initiated within a few days after infection by rapid selection of mutants resistant to host immune response, resulting in the development of reservoirs of progeny virus within one to two weeks after infection (Allen et al., 2005; Allen et al., 2004; Jones et al., 2009; Rychert et al., 2007; Salazar-Gonzalez et al., 2009). Changes in the proteins of the escape mutants, even of single amino acids, can result in loss of T-cell epitopes by modification of sequences required at any of several stages in the immune response mechanisms; for example, antigen protein processing of T-cell epitope sequences, epitope recognition by human leukocyte antigen (HLA), or epitope ligation and activation of T-cell receptors (Allen et al., 2004; Draenert et al., 2004; Kelleher et al., 2001; Leslie et al., 2004; Sloan-Lancaster and Allen, 1996; Yokomaku et al., 2004). Escape from the immune response is, however, limited in some individuals (HIV-controllers) and a recent report provides extensive genetic data implicating HLA-viral peptide interaction as the major factor in the control of HIV infection by these individuals (Pereyra et al., 2010). The ability of HIV-1 to escape the host immune system via mutation may also be restricted at sites of the genome (Korber et al., 2009; Yang, 2009) important for viral functions. Vaccines that target certain conserved epitopes of virus structural and regulatory proteins have been shown to elicit cellular immune responses that provide immune protection against HIV infection in BALB/c and transgenic mice (Gotch, 1998; Korber et al., 2009; Letourneau et al., 2007; Okazaki et al., 2003; Wilson et al., 2003).

There is a continuing need in the art for effective diagnosis, vaccines and treatments for HIV.

### SUMMARY OF THE INVENTION

According to one aspect of the invention a polypeptide comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database. Two of these polypeptides are specific to HIV-1, with no matching sequence of nine amino acids in the sequences of other viruses or organisms reported in nature (as of December 2010), while many are specific to primate lentivirus group, including HIV-1 with multiclade conservation of the following possible combinations: clades A, B, C and D or clades B, A, and C or clades B, A and D or clades B, C and D or clades B and A or clades B and C or clades B and D or clade B only. The multiclade sequences may be used to specifically identify HIV-1 virus of the different clades.

Another aspect of the invention is a polynucleotide encoding the polypeptide that comprises one or more discontinuous segments of HIV-1 clade B proteins. The segments comprise from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

Yet another aspect of the invention is a polypeptide made from an encoding polynucleotide, that further comprises: (a) a LAMP-1 luminal sequence comprising SEQ ID NO: 1278; and (b) a LAMP transmembrane and cytoplasmic tail comprising SEQ ID NO: 1279, wherein the luminal sequence is amino-terminal to the one or more discontinuous segments of the HIV-1 proteins which are amino-terminal to the LAMP transmembrane and cytoplasmic tail.

Additionally, a nucleic acid vector is provided that comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

Further, a host cell is provided that comprises a nucleic acid vector that comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

Another aspect of the invention is a method of producing a polypeptide. A host cell is cultured under conditions in which the host cell expresses the polypeptide. The host cell comprises a nucleic acid vector that comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

A method is provided for producing a cellular vaccine. Antigen presenting cells are transfected with a nucleic acid vector, whereby the antigen presenting cells express the polypeptide. The nucleic acid vector comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

A method of making a vaccine is another aspect of the invention. The method comprises mixing together a polypeptide and an immune adjuvant. The polypeptide comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

A method of immunizing a human or other animal subject is another aspect of the invention. The method comprises administering to the human or other animal subject a polypeptide or a nucleic acid vector or a host cell, in an amount effective to elicit HIV-specific T-cell activation. The polypeptide comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database. The nucleic acid vector comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database. The host cell comprises a nucleic acid vector that comprises the polynucleotide that encodes a polypeptide that comprises one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

Additional aspects of the invention permit the identification of lentivirus group, species, or clade. Oligonucleotide probes hybridize to genomic nucleic acid or its complement and identify group, species, or clade.

Another aspect of the invention involves protein-based diagnosis. A polypeptide which represents a conserved sequence according to the invention or an antibody which specifically binds such a conserved sequence is used to interrogate by binding a body sample of a patient. An antibody is used to identify viral protein in virus infected cells. A polypeptide is used to identify a patient's own antibodies to a lentivirus. Specific binding can be used to identify the presence in the patient of the primate lentivirus group species, including the HIV-1 species, of a specific clade, biclade, triclade or pan-clade.

These and other embodiments, which will be apparent to those of skill in the art upon reading the specification provide the art with methods and tools for reducing risk, severity, symptoms, and/or duration of acquired immunodeficiency disease. Thus the vaccines may be either prophylactic or therapeutic.

### BRIEF DESCRIPTION OF THE DRAWINGS

FIG. 1 shows Shannon's nonamer entropy of the HIV-1 clade B proteins.

FIG. 2 shows density plots of the incidence of total variants of the primary nonamer and the entropy of the nonamer sequences of clade B proteins.

FIG. 3 shows density plots of the incidence (%) of all variants to the primary nonamer and the primary variant at each nonamer position of the HIV-1 clade B proteins. The regions boxed in red and the adjacent values indicate the fraction and number of total nonamer positions analyzed that are highly conserved and contain fewer than 20% variants of the primary sequence and fewer than 10% incidence of the primary variant. nonamer sequences of each protein.

### DETAILED DESCRIPTION OF THE INVENTION

The inventors have identified and selected polypeptides that represent epitopes in humans, which are conserved in at least 80% of all recorded HIV clade B viruses as of August 2008, and wherein individual variants have an incidence of less than 10%. Selection criteria may be increased in stringency to, for example at least 85% or 90% or 95% incidence of primary conserved sequence and decreased individual variant stringency to an incidence of less than 5% or 1%. These epitopes are useful for vaccines as well as for diagnostic assays.

Discontinuous segments of the HIV-1 may be strung together to form a concatamer, if desired. They may be separated by spacer residues. Discontinuous segments are those that are not adjacent in the naturally occurring virus isolates. Segments are typically at least 9 amino acid residues and up to about 15, 16, 17, 18, 19, 20, 25, 30, 35, or 40 residues of contiguous amino acid residues from the virus proteome. Single segments may also be used. Because the segments are less than the whole, naturally occurring proteins, and/or because the segments are adjacent to other segments to which they are not adjacent in the proteome, the polypeptides and nucleic acids described here are non-naturally occurring.

Linkers or spacers with natural or non-naturally occurring amino acid residues may be used optionally. Particular properties may be imparted by the linkers. They may provide a particular structure or property, for example a particular kink or a particular cleavable site. Design is within the skill of the art.

Polynucleotides which encode the polypeptides may be designed and made by techniques well known in the art. The natural nucleotide sequences used by HIV-1 may be used. Alternatively non-natural nucleotide sequences may be used, including in one embodiment, human codon-optimized sequences. Design of human codon-optimized sequences is well within the skill of the ordinary artisan. Data regarding the most frequently used codons in the human genome are readily available. Optimization may be applied partially or completely.

The polynucleotides which encode the polypeptides can be replicated and/or expressed in vectors, such as DNA virus vectors, RNA virus vectors, and plasmid vectors. Preferably these will contain promoters for expressing the polypeptides in human or other mammalian or other animal cells. An example of a suitable promoter is the cytomegalovirus (CMV) promoter. Promoters may be inducible or repressible. They may be active in a tissue specific manner. They may be constitutive. They may express at high or low levels, as desired in a particular application. The vectors may be propagated in host cells for expression and collection of chimeric protein. Suitable vectors will depend on the host cells selected. In one embodiment host cells are grown in culture and the polypeptide is harvested from the cells or from the culture medium. Suitable purification techniques can be applied to the chimeric protein as are known in the art. In another embodiment one transfects antigen-presenting cells for ultimate delivery of the transfected cells to a vaccinee of a cellular vaccine which expresses and presents antigen to the vaccinee. Suitable antigen presenting cells include dendritic cells, B cells, macrophages, and epithelial cells.

Polynucleotides of the invention include diagnostic DNA or RNA oligonucleotides, i.e., short sequences of proven specificity to viral species; these are sufficient to uniquely identify the viral species or to a group or clade (SEQ ID NOs: 637-1140). Polynucleotides include oligonucleotides such as primers and probes, which may be labeled or not. These may contain all or portions of the coding sequences for an identified conserved polypeptide. Polynucleotides of the invention and/or their complements, may optionally be attached to solid supports as probes to be used diagnostically, for example, through hybridization to viral genomic sequences. Similarly, epitopic polypeptides can be attached to solid supports to be used diagnostically. These can be used to screen for activated T cells or even antibodies. Suitable solid supports include without limitation microarrays, microspheres, and microtiter wells. Antibodies may be used that are directed against the peptides as disclosed. The antibodies may be used to specifically diagnose species of the primate lentivirus group, including HIV-1 virus with multiclade conservation of the following possible combinations: clades A, B, C and D or clades B, A, and C or clades B, A and D or clades B, C and D or clades B and A or clades B and C or clades B and D or clade B only. The multiclade sequences may be used to specifically identify HIV-1 virus of the different clades. Polynucleotides may also be used as primers, for example, of length 18-30, 25-50, or 15-75 nucleotides, to amplify the genetic material of viruses of the primate lentivirus group, including HIV-1 virus(es) of the possible clade combinations listed above. Polynucleotide primers and probes may be labeled with a fluorescent or radioactive label, if desired. These polynucleotides can be used to amplify and/or hybridize to a test sample to determine the presence or species identity of a primate lentivirus, including HIV-1 virus(es) of the possible clade combinations listed above. Such polynucleotides will typically be at least 15, 18, 20, 25, or 30 bases to 50, 70, 90, 120, 150, or 500 bases in length. Any technique, including but not limited to amplification, hybridization, single nucleotide extension, and sequencing, can be used to identify the presence or species identity of the primate lentivirus, including HIV-1 virus(es) of the possible clade combinations listed.

Immune adjuvants may be administered with the vaccines of the present invention, whether the vaccines are polypeptides, polynucleotides, nucleic acid vectors, or cellular vaccines. The adjuvants may be mixed with the specific vaccine substance prior to administration or may be delivered separately to the recipient, either before, during, or after the vaccine substance is delivered. Some immune adjuvants which may be used include CpG oligodeoxynucleotides, GM-CSF, QS-21, MF-59, alum, lecithin, squalene, and Toll-like receptors (TLRs) adaptor molecules. These include the Toll-interleukin-1 receptor domain-containing adaptor-inducing beta interferon (TRIF) or myeloid differentiation factor 88 (MyD88). Vaccines may be produced in any suitable manner, including in cultured cells, in eggs, and synthetically. In addition to adjuvants, booster doses may be provided. Boosters may be the same or a complementary type of vaccine. Boosters may include a conventional live or attenuated HIV-1 vaccine. Typically a high titer of antibody and/or T cell activation is desired with a minimum of adverse side effects.

Any of the conventional or esoteric modes of administration may be used, including oral, mucosal, or nasal. Additionally intramuscular, intravenous, intradermal, or subcutaneous delivery may be used. The administration efficiency may be enhanced by using electroporation. Optimization of the mode of administration for the particular vaccine composition may be desirable. The vaccines can be administered to patients who are infected already or to patients who do not yet have an infection. The vaccines can thus serve as prophylactic or therapeutic agents. One must, however, bear in mind that no specific level of efficacy is mandated by the words prophylactic or therapeutic. Thus the agents need not be 100% effective to be vaccines. Vaccines in general are used to reduce the incidence in a population, or to reduce the risk in an individual. They are also used to stimulate an immune response to lessen the symptoms and or severity of the disease.

The above disclosure generally describes the present invention. All references disclosed herein are expressly incorporated by reference. A more complete understanding can be obtained by reference to the following specific examples, which are provided herein for purposes of illustration only, and are not intended to limit the scope of the invention.

### EXAMPLES

We conducted a large-scale, systematic analysis of the recorded HIV-1 clade B protein sequences, focused on the variability and conservation of T-cell epitope relevant sequences. Detailed analyses were performed with clade B as it has the largest number of recorded sequences and can be used as a model for similar studies of the other clades. Modified Shannon's entropy and bioinformatics approaches were used to measure nonamer conservation and variability. Nonamers were chosen as they are the typical length of HLA class I epitopes, and the cores of HLA class II epitopes (Rammensee, 1995). Variants of the conserved nonamer sequences were analysed for the identification of regions of the proteome that were not only conserved, but also had a low incidence of individual variants. The immune relevance of selected sequences was assessed by their correlation with previously reported human T-cell epitopes and our recent study in the identification of human HIV-1 T-cell epitopes by use of HLA transgenic mice (Simon et al., 2010). The studies also included the identification of a) sequences specific to HIV-1 with no shared identity to other viruses and organisms, and b) specific sequences that are multiclade conserved as vaccine targets. These sequences have direct relevance to the development of new-generation vaccines and diagnostic applications.

### Example 1

### Materials and Methods

### Data Preparation, Selection and Alignment of HIV-1 Clade B Protein Sequences

HIV-1 protein sequence records were retrieved from the NCBI Entrez Protein Database in August 2008 by searching the NCBI taxonomy browser for HIV-1 (Taxonomy ID 11676). HIV-1 clade B specific entries were retrieved from the data collected via BLAST (version 2.2.18) searches (Altschul et al., 1990), using default parameters, with sample HIV-1 clade B protein sequences of the nine HIV-1 proteins from the HIV database (see website of Los Alamos National Laboratory (LANL) for HIV) as queries. Cutoff for the classification of each clade B protein was determined by manual inspection of the individual BLAST outputs. Duplicate sequences of each protein were removed and the remaining unique sequences, both partial and full length, were used for protein multiple sequence alignment. Alignment was difficult for some of the proteins because of the large number of diverse sequences, and thus different approaches were explored, as described below.

Sequence alignments of Vif, Vpr and Vpu were performed with PROMALS3D (Pei et al., 2008). The Gag, Pol, Tat, Rev, Env and Nef protein sequence with large datasets were first split into smaller and more manageable sections (about 200-500 sequences per subset). These smaller subsets were aligned using PROMALS3D or CLUSTAL W (Pei et al., 2008; Thompson et al., 1994) and refined with RASCAL (Thompson et al., 2003) before merging into a full protein multiple sequence alignment, by use of conserved sites that helped anchor the alignment subsets. All multiple sequence alignments were manually inspected and corrected for misalignments. Alignment positions with high fraction of gaps, 95% or more were removed. In total 29,211 Env protein sequences were retrieved but only 9,661 sequences were aligned and analysed due to the complexity in aligning large diverse protein sequences.

Protein alignment positions of clade B were cross-referenced to the HXB2 prototype protein sequences. It should be noted that the protein alignment positions differ from the HXB2 positions due to insertions and deletions in the alignment, especially in regions of high diversity.

### Nonamer Diversity and Conservation of HIV-1 Clade B Proteins

Shannon's entropy (Miotto et al., 2008; Shannon, 1948) was used as a measure for HIV-1 diversity. The entropy of all overlapping nonamer positions across the protein alignment of HIV-1 clade B was measured and plotted by use of the ggplot2 suite (Wickham, 2009) of the R programming language and environment (R_Development_Core_Team, 2008). Entropy analysis was carried out by use of the Antigenic Variability Analyser tool (AVANA; see sourceforge website) and following the method as described in Khan et al. (2008). Briefly, the computation of entropy involves the number and incidence of unique nonamer peptides at a given position in an alignment. Nonamer entropy H(x) for a given position x in the alignment was calculated using the formula:

$$H ( x ) = - {\sum }_{i = 1}^{n ( x )} {p}_{i , x} {log}_{2} ( {p}_{i , x} )$$

where p_{i,x} is the probability of the occurrence (or incidence) of nonamer i with its center position at x (also referred to as the “nonamer position”), and n(x) is the total number of unique peptides observed at position x. Since the entropy values were calculated for each nonamer window based on its center position, values were not assigned to the four amino acids at the beginning and end of the alignments. A position that has a large number of unique peptides with majority displaying high incidence would evaluate to a high entropy value, which would imply that this position is highly diverse, where the maximum nonamer entropy value possible is 39 (log₂20⁹). Conversely, if the position has a single peptide that is completely conserved across all the sequences at that position in the alignment, the entropy will be zero, the lowest value possible. Entropy calculations are affected by the size of an alignment, and hence the entropies within the protein alignments of HIV-1 clade B were corrected for size bias via a statistical sub-sampling method (Khan et al., 2008).

### Distribution of Nonamer Variants Across HIV-1 Clade B Proteome

All sequences at each of the nonamer positions in the protein alignments were extracted and studied for the incidence of the primary (most common) nonamer and its variants. Variants at a given position in the alignment were defined as peptides with at least one amino acid difference from the primary nonamer. Variant nonamers that contained gaps (−) or any one of the unresolved characters, including B (asparagine or aspartic acid), J (leucine or Isoleucine), X (unspecified or unknown amino acid) and Z (glutamine or glutamic acid) were excluded from the analysis. The ggplot2 suite was used to depict the incidence of total nonamer variants and the primary variant at each nonamer position across the proteome.

### Identification of Highly Conserved Sequences in HIV-1 Clade B Proteins

Highly conserved HIV-1 clade B sequences were identified as nonamers positions with (i) a primary nonamer incidence of 80% or more of the analysed viral sequences at that position and (ii) incidence of the primary variant of less than 10% of the primary nonamer sequence at the position. Identified nonamers that were contiguous (overlapped by eight amino acids) were joined. Positions with less than 100 sequences in the alignment were excluded from the selection of conserved sequences.

Correspondence of Highly Conserved HIV-1 Clade B Sequences with Reported T-Cell Epitopes

All published human T-cell epitopes from the HIV Molecular Immunology Database (November 2010) (see website of Los Alamos National Laboratory (LANL) for immunology) and our transgenic mice study (Simon et al., 2010) with a match of at least 9 consecutive amino acids with the highly conserved HIV-1 clade B sequences were identified.

### Identification of HIV-1 Pan-Clade and Biclade Highly Conserved Sequences

The 2008 Web alignment of the complete protein sequences of the HIV-1 clade A, C and D were obtained from HIV sequence database (see website of Los Alamos National Laboratory (LANL) for HIV). All protein alignments were manually inspected and corrected where necessary. The clade B highly conserved sequences were analysed for their incidence in the corresponding protein alignments of clade A, C and D to identify HIV-1 pan-clade highly conserved sequences. Highly conserved HIV-1 sequences common to clade B and C were also identified as there was limited data for most of the proteins of clade A and D. The criteria for identification of pan-clade and biclade highly conserved sequences was similar to that used for clade B. Identified pan-clade and biclade nonamers that were contiguous were joined to form longer sequences.

### Identification of Highly Conserved HIV-1 Clade B Sequences Common to Other Viruses and Organisms

Highly conserved HIV-1 clade B sequences that overlapped at least 9 consecutive amino acids sequences of other viruses and organisms were identified by performing an exhaustive string search of the nonamers of the conserved sequences against all protein sequences reported at the NCBI Entrez protein database (as of November 2010), excluding HIV-1 records, synthetic constructs and artificial sequences.

### Example 2

### HIV-1 Clade B Protein Datasets and Protein Alignment

A total of 58,052 sequences of the HIV-1 clade B proteome, over 1000 of each protein, were extracted from the NCBI Entrez Protein Database and aligned for the analysis of the evolutionary conservation and diversity (Table 1). Approximately 90% or the sequences were of the Gag, Pol, Env, and Nef proteins. The other 5 proteins almost equally shared the remaining 6513 sequences. Sequences of other clades were obtained from the HIV Sequence Database Web alignment. The clade C alignment contained almost 4000 sequences, between 300 and 600 of each protein. Clades A and D had few sequences. Duplicate sequences, either partial or full-length, were removed to eliminate the possible bias of redundant sequences derived from identical HIV-1 isolates sequenced by surveillance programs or large sequencing projects at specific sites.

TABLE 1
HIV-1 sequences analysed.

| | Amino | Number of sequences | | | |
|---|---|---|---|---|---|
| Protein | Acids^{a} | Clade A^{b} | Clade B^{c} | Clade C^{b} | Clade D^{b} |
| Gag | 500 | 150 | 6,403 | 591 | 77 |
| Pol | 1,003 | 64 | 30,604 | 384 | 47 |
| Vif | 192 | 91 | 1,147 | 375 | 49 |
| Vpr | 78 | 61 | 1,041 | 425 | 45 |
| Tat | 86 | 66 | 1,569 | 304 | 44 |
| Rev | 116 | 64 | 1,533 | 336 | 46 |
| Vpu | 81 | 197 | 1,223 | 418 | 73 |
| Env | 856 | 102 | 9,661 | 510 | 85 |
| Nef | 206 | 150 | 4,871 | 558 | 98 |

| | | | | |
|---|---|---|---|---|
| Total | 945 | 58,052 | 3,901 | 564 |

^{a}Approximate size with respect to HXB2 sequences.
^{b}Retrieved from HIV Sequence Database Web alignment. Sequences are used for the identification of HIV-1 pan-clade sequences. Refer to materials and methods for more information.
^{c}Retrieved from NCBI Entrez Protein Database

### Example 3

### Nonamer Peptide Conservation and Diversity

Shannon's entropy methodology, commonly applied to measure differences in single amino acid residues in the alignment of protein sequences, was modified to analyze each of the 3,133 nonamer positions, overlapping by eight amino acids, that represent all putative MHC binding cores of the of the HIV-1 clade B proteome. The average number of each of the nonamer sequences at a given protein position depended on the alignment of the sequences taken from the NCBI Entrez Protein Database, ranging from an average of 965 aligned sequences for Vpr and Vpu, to 5,558 for Pol (Table 2). Entropy of a nonamer sequence results from change of one or more of the 20 amino acids at a single site or at multiple sites of the 9 amino acid nonamer unit, with a maximum entropy of 39 if there were all possible changes of each amino acid (log₂20⁹). Because these units are overlapping, an amino acid at the 9^{th} position will eventually move to the 1^{st} as the nonamer units shift from the N- to the C-terminus. Thus, a single variant amino acid is commonly seen in 9 overlapping nonamer sequences and the diversity of a series of nonamer units with one or more variant amino acids is typically clustered.

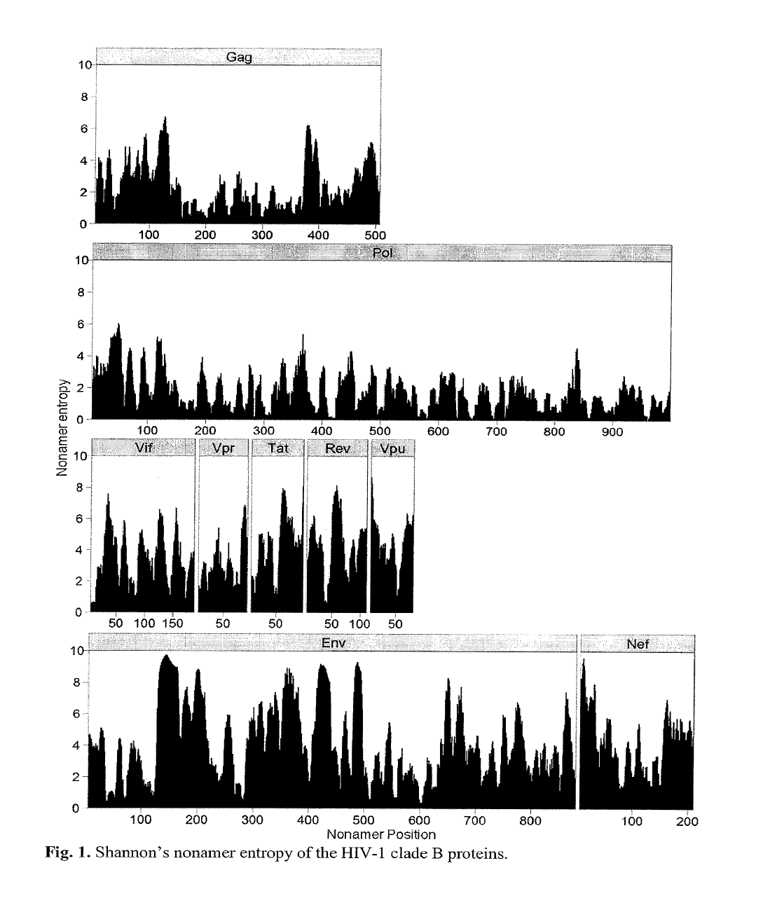

The extraordinary evolutionary diversity of HIV-1 proteins was evident from the range in the entropy of the overlapping nonamer units (FIG. 1). Each of the proteins had discrete regions of highly conserved nonamer sequences with entropy less than 1.0, and regions of extreme diversity, some with entropy approaching 10.0, the highest we have documented, relative to influenza (Heiny et al., 2007), dengue (Khan et al., 2008) and West Nile virus (Koo et al., 2009). Highly conserved nonamers were present chiefly in Pol, distributed throughout the protein with an average nonamer entropy of 1.8, and in Gag, localized in the middle of the protein between amino acid positions 170 to 370 with an average entropy of 2.4 (Table 2). The only completely conserved nonamer sequences, entropy 0.0, of the entire clade B proteome were three in Pol (710-718, 956-964, and 957-965). While Env, with an average nonamer entropy of 4.2, is commonly considered the most diverse HIV-1 protein, each of the nonstructural proteins Tat, Rev, Vpu, and Nef also had multiple sequences with high nonamer entropies, with an average range of 4.3 to 4.6.

The data of each nonamer sequence of the protein alignments quantitatively document the incidence (prevalence) of the primary nonamer, total variants of the primary nonamer, primary variant and number of unique variants (Table 2).

All nonamer positions (3133) of the aligned clade B database sequences were compared with the clade B consensus HXB2 sequence. Many of the HXB2 sequences as expected were identical to the aligned database sequences. However, the HXB2 sequences represent selected variant strain and differ markedly at many positions from the primary nonamers of the aligned database sequences, especially in regions of high diversity.

An example of highly conserved and highly variable nonamer sites are the 25 overlapping nonamer positions of Env114-122:140-148 (Table 3). The five sites of the Env114-122:118-126 were highly conserved, with entropies of 0.8 to 1.1, containing primary nonamer sequences identical to those of HXB2 and with an incidence of 86 to 89% of the ˜1000 to 1600 aligned nonamer sequences at each of these sites. The remaining ˜11% to 15% of the aligned nonamers of these conserved Env sites were variants of the primary nonamer, comprising 21 to 29 unique sequences, with a 4 to 6% incidence of the primary (most common) variant of all nonamers analysed per site. Beginning at position Env119-127 the sequence diversity increased with amino acids that differed at some sites from almost every amino acid of HXB2, nonamer entropy increased to as high at 9.8, and primary nonamer sequences represented as few as 49 (˜2%) of the over 3000 nonamers at each of these aligned positions. Practically all of the nonamer sequences at these highly diverse sites of Env were variants of the primary sequence, with over 1000 unique sequences and fewer than 100 of the primary variant sequence at any one position.

TABLE 2
Summary, nonamer conservation and diversity analysis of the HIV-1 clade B proteome.

| | | Average | | | Variants of the primary nonamer^{f} | | |
|---|---|---|---|---|---|---|---|
| | | nonamer | | | | | Average |
| | Total | sequences | | | | | primary |
| | nonamer | analysed per | Average | Primary nonamer^{d} | Average total | Average | variant |
| | positions^{a} | position^{b} | Nonamer | Average incidence^{e} | incidence^{g} | unique^{h} | incidenceⁱ |
| Protein | [No.] | [No.] | Entropy^{c} | [No. (%)] | [No. (%)] | [No.] | [No. (%)] |
| Gag | 504 | 1628 | 2.4 | 967 (~59) | 669 (~41) | 72 | 243 (~15) |
| Pol | 995 | 5558 | 1.8 | 3902 (~70) | 1649 (~30) | 90 | 604 (~11) |
| Vif | 184 | 1132 | 3.5 | 511 (~45) | 620 (~55) | 97 | 149 (~13) |
| Vpr | 88 | 965 | 3.1 | 497 (~51) | 471 (~49) | 81 | 136 (~14) |
| Tat | 94 | 1078 | 4.3 | 451 (~42) | 627 (~58) | 138 | 105 (~10) |
| Rev | 108 | 1220 | 4.4 | 444 (~37) | 769 (~63) | 142 | 142 (~12) |
| Vpu | 77 | 965 | 4.6 | 368 (~38) | 596 (~62) | 130 | 114 (~12) |
| Env | 877 | 2100 | 4.2 | 764 (~36) | 1335 (~64) | 196 | 224 (~11) |
| Nef | 206 | 3972 | 4.3 | 1529 (~39) | 2431 (~61) | 247 | 564 (~14) |

^{a}Note that the total number of nonamer positions analysed is different from the number of amino acids from the HXB2 sequences due to insertions and deletions in the protein alignments.
^{b}Average number of sequences analysed at each nonamer position (1-9, 2-10, 3-11, etc) of the protein alignments. The number of sequences varies due to the inclusion of both partial and full-length sequences.
^{c}Average Shannon's nonamer entropy across all nonamer positions in the protein alignment. For example, the average Gag Shannon's entropy is the mean entropy across all 504 nonamer positions in the Gag protein alignment.
^{d}The primary nonamer is the peptide with the highest incidence at a given nonamer position in the protein alignment.
^{e}Average incidence of the primary (most frequent) nonamer across all the positions in the protein alignment.
^{f}Variants of the primary nonamers are all sequences that differ by one or more amino acids from the primary nonamer at a given nonamer position in the protein alignment.
^{g}Average incidence of the variants of the primary nonamer in the protein alignments.
^{h}Average number of different variant sequences to the primary nonamer.
ⁱAverage incidence of the primary variant nonamer, the most highly represented variant sequence of all the nonamers analysed per nonamer position in the protein alignments.

TABLE 3
Example of nonamer conservation and diversity with a selected highly conserved and
highly diverse region of HIV-1 clade B Env protein*^{#}. SEQ ID NOs: 1-27 for HXB2
in the order as shown and 28-54 for the primary nonamer of HIV-1 clade B Env
sequences in the order as shown.

| | | | | | | | Variants of the primary | | |
|---|---|---|---|---|---|---|---|---|---|
| | | | | | | | nonamers ^{e} | | |
| | | | | | | | | | Primary |
| | | | | | | | | | variant |
| | Nonamers analysed | | | HXB2 | Primary nonamer ^{d} | | Total | | inci- |
| Pro- | | | Nonamer | Nonamer | | Incidence | incidence | Unique ^{f} | dence ^{g} |
| tein | Position | No. ^{a} | entropy ^{b} | Sequence ^{c} | Sequence ^{d} | [No. (%)] | [No. (%)] | [No.] | [No. (%)] |
| Env | 114-122 | 1032 | 1.0 | SLKPCVKLT | ......... | 884 (~86) | 148 (~14) | 21 | 61 (~6) |

| | | | | | | | | |
|---|---|---|---|---|---|---|---|---|
| 115-123 | 1034 | 1.0 | LKPCVKLTP | ......... | 887 (~86) | 147 (~14) | 21 | 61 (~6) |
| 116-124 | 1066 | 1.1 | KPCVKLTPL | ......... | 904 (~85) | 162 (~15) | 29 | 60 (~6) |
| 117-125 | 1517 | 0.8 | PCVKLTPLC | ......... | 1357 (~89) | 160 (~11) | 29 | 59 (~4) |
| 118-126 | 1568 | 0.8 | CVKLTPLCV | ......... | 1397 (~89) | 171 (~11) | 29 | 83 (~5) |
| 119-127 | 1594 | 1.1 | VKLTPLCVS | ........T | 1374 (~86) | 220 (~14) | 33 | 83 (~5) |
| 120-128 | 2665 | 1.0 | KLTPLCVSL | .......T. | 2341 (~88) | 324 (~12) | 50 | 101 (~4) |

| | | | | | | | | |
|---|---|---|---|---|---|---|---|---|
| 121-129 | 2670 | 1.7 | LTPLCVSLK | ......T.N | 2037 (~76) | 633 (~24) | 63 | 142 (~5) |
| 122-130 | 3112 | 1.9 | TPLCVSLKC | .....T.N. | 2313 (~74) | 799 (~26) | 73 | 151 (~5) |
| 123-131 | 3326 | 2.7 | PLCVSLKCT | ....T.N.. | 2195 (~66) | 1131 (~34) | 120 | 146 (~4) |
| 124-132 | 3368 | 4.1 | LCVSLKCTD | ...T.N... | 1488 (~44) | 1880 (~56) | 200 | 425 (~13) |
| 125-133 | 3673 | 6.3 | CVSLKCTDL | ..T.N...N | 455 (~12) | 3218 (~88) | 388 | 353 (~10) |
| 126-134 | 3675 | 7.6 | VSLKCTDLK | .T.N...NW | 353 (~10) | 3322 (~90) | 555 | 116 (~3) |
| 127-135 | 3677 | 8.2 | SLKCTDLKN | T.N...NW. | 348 (~9) | 3329 (~91) | 697 | 108 (~3) |
| 128-136 | 3719 | 8.7 | LKCTDLKND | .N...NW.N | 227 (~6) | 3492 (~94) | 813 | 78 (~2) |
| 129-137 | 3725 | 8.9 | KCTDLKNDT | N...NW.N. | 227 (~6) | 3498 (~94) | 900 | 78 (~2) |
| 130-138 | 3912 | 9.1 | CTDLKNDTN | ...NW.N.G | 216 (~6) | 3696 (~94) | 981 | 79 (~2) |
| 131-139 | 3917 | 9.2 | TDLKNDTNT | ..NW.N.GN | 210 (~5) | 3707 (~95) | 1051 | 79 (~2) |
| 132-140 | 3911 | 9.4 | DLKNDTNTN | .NW.N.GNV | 199 (~5) | 3712 (~95) | 1098 | 78 (~2) |
| 133-141 | 3863 | 9.5 | LKNDTNTNS | NW.N.GNV. | 196 (~5) | 3667 (~95) | 1133 | 78 (~2) |
| 134-142 | 3838 | 9.5 | KNDTNTNSS | W.N.GNV.D | 175 (~5) | 3663 (~95) | 1156 | 78 (~2) |
| 135-143 | 3807 | 9.5 | NDTNTNSSS | .N.GNV.D. | 173 (~5) | 3634 (~95) | 1166 | 85 (~2) |
| 136-144 | 3747 | 9.6 | DTNTNSSSG | N.GNV.D.S | 169 (~5) | 3578 (~95) | 1194 | 87 (~2) |
| 137-145 | 3701 | 9.7 | TNTNSSSGR | .GNV.D.SW | 179 (~5) | 3522 (~95) | 1191 | 89 (~2) |
| 138-146 | 3473 | 9.7 | NTNSSSGRM | GNV.D.SWK | 144 (~4) | 3329 (~96) | 1166 | 42 (~1) |
| 139-147 | 3090 | 9.7 | TNSSSGRMI | .SVN.NSSG | 49 (~2) | 3041 (~98) | 1070 | 42 (~1) |
| 140-148 | 2699 | 9.8 | NSSSGRMIM | SVN.NSSGG | 49 (~2) | 2650 (~98) | 968 | 39 (~1) |

^{a} The total number of HIV-1 clade B protein sequences obtained at the respective nonamer positions of the protein sequence alignment. The number of sequences for each nonamer position varies due to the inclusion of both partial and full-length sequences.
^{b} Shannon's nonamer entropy.
^{c} The nonamer sequence corresponding to the HXB2 reference sequence. Insertions to the alignment with respect to the HXB2 sequence are shown as gaps “-”.
^{d} The primary nonamer is the peptide with the highest incidence at a given nonamer position in the protein alignment. Residues that are identical to the HXB2 sequence is denoted as “.” whereas residues that are different have their amino acids displayed. For example, at position 1-9 of Gag, the HXB2 sequence have identical sequence to that of the primary nonamer thus the primary nonamer have the sequence “.........” displayed. However at position, 22-30 in Gag, the last residue in the nonamer differs from that of HXB2, having R instead of K, and thus the nonamer sequence is shown as “........R”.
^{e} Variants of the primary nonamers are all sequences that differ by one or more amino acids from the primary nonamer at the corresponding position in the protein alignment.
^{f} The number of unique variants at the indicated nonamer position.
^{g} The primary variant is the most common (highest incidence) variant nonamer at the indicated nonamer position of the protein alignment.
^{f} The primary variant is the most common variant nonamer at the indicated nonamer position of the protein alignment.
*An example interpretation of the table: The primary nonamer SLKPCVKLT was present in 884 sequences (~86%) of all 1032 sequences analyzed at nonamer position 114-122 in the Env protein alignment. The remaining 148 sequences (~14%) at that position were variants of the primary nonamer and comprised 21 unique peptides, one of which is the primary variant and is present in about 6% (61) of all the 1032 analysed sequences. The remaining 20 variants at that position were represented by 87 additional variant sequences.
^{#}This example shows a region of low entropy, positions 114-128 with entropy below 1.1, which is connected to positions 127-148, a region of high diversity (entropy above 8.0), by a transitional region of intermediate entropy.

### Example 4

### Limited Nonamer Positions Across the HIV-1 Proteome with Low Total Variants Incidence

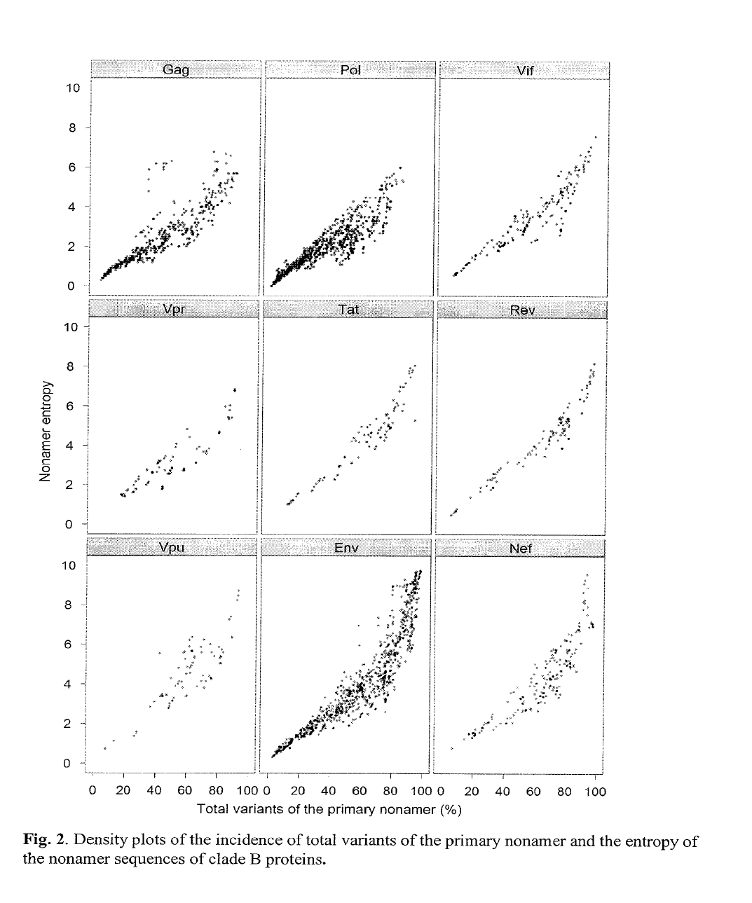

A possible criterion for effective HIV-1 vaccine design is the consideration of the incidence of total variants to the primary nonamer. The total variants at each nonamer position represent the population of possible altered ligands that the immune system maybe exposed to upon immunization with the most common or primary nonamer at the position. We thus analysed the distribution of total variants of the primary nonamer in the context of diversity across the entire HIV-1 proteome (FIG. 2) as measured by use of entropy. All the proteins, even Pol, Gag and Vpr with high average primary nonamer incidence (Table 2), included numerous positions with total variants incidence as high as >80%. This was particularly so for the Env where more than 228 of the 877 (26%) nonamer positions analysed exhibited a total variants incidence of >80%, with a maximum of 98%. Even though there was a general pattern of entropy increasing as total variants increased, exceptions exist as positions with high total variants (more than 27% and up to 59%) incidence were also observed for all proteins when the entropy was low <2.0. Although entropy is a good measure for diversity, it is alone not sufficient for selection of conserved positions with low total variants for identification of vaccine targets. Only a small fraction of the nonamer positions of all the HIV-1 proteins (493 of 3133, ˜16%) exhibited total variants of <20%. This highlights the importance of detailed analysis of HIV-1 diversity for careful rational selection of the limited desired sites for vaccine design. This also suggests that existing HIV-1 vaccine approaches that do not consider the variant populations for selection of targets may exhibit limited efficacy.

### Example 5

### Influence of Primary Variants at Positions with Low Total Variants Incidence

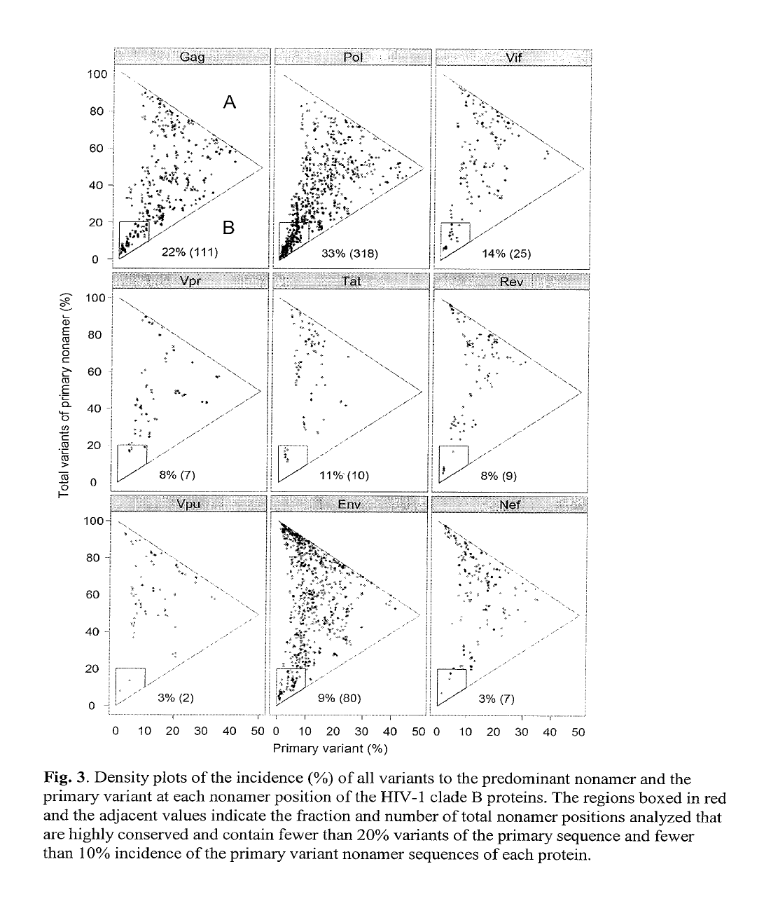

Highly conserved positions with low total variants (<20%) are attractive sites for selection of vaccine targets, however, such sites with a large proportion of the total variants dominated by a single primary variant should be avoided. Analysis of the incidence of primary variants for all nonamer positions across the HIV-1 proteome (FIG. 3) revealed that as total variant incidence increases there is a wide range in the fraction of the primary variant, from about <1% to a maximum incidence up to 45%, with more than 40% incidence in Gag (3 positions, <1% of all positions), Pol (19 positions, ˜2%), and Env (5 positions, <1%). The shape of the plot depicts the increasing incidence of the primary variant to a maximum limited by the incidence of the total variants (zone A in the plot), after which (>50% total variant incidence) the incidence of the primary variant is further limited by the decreasing incidence of the primary nonamer (zone B), because the primary variant, the second most common peptide at a nonamer position, cannot exceed the incidence of the most common primary nonamer. Highly conserved sites with less than 20% total variants had individual primary variants with an incidence of more than 10% in Gag (15%), Pol (14%), Env (12%) and Nef (12%). The primary nonamer of low total variant sites (<20%) with major variant of <10% are attractive targets for HIV-1 vaccine design, and were identified and joined where possible (termed as highly conserved HIV-1 Clade B sequences). This comprised for Gag, 22% or 111 of 504 total primary nonamers; Pol, 33%, 318 of 995; Vif, 14%, 25 of 184; and Env 9%, 80 of 887 (red enclosed region in FIG. 3). The remainder of the HIV-1 proteins had fewer than 11% of the total primary nonamers of the proteins that conformed to these criteria.

### Example 6

### Clade B HIV-1 Protein Sequences of Nine or More Amino Acids that are Highly Conserved (Incidence of 80% or More) with Less than 10% Primary Variant Incidence

A total of 78 highly conserved HIV-1 Clade B sequences (504 total nonamers) were identified across the whole proteome (Table 4 and Table 5). The length of these peptides ranged from 9 to 40 amino acids, covering a total length of 1101 amino acids (˜35%) of the complete HIV-1 proteome (˜3133 aa). The structural (Env and Gag) and enzymatic (Pol) proteins contained the greatest number of conserved sequences. Pol, the most conserved HIV-1 clade B protein with the lowest average nonamer entropy of 1.8 and lowest average total variants incidence of about 30% (Table 2), had 31 conserved sequences covering ˜48% of the protein length. The relatively more conserved Gag and the highly variable Env had 18 (˜51% of the protein length) and 14 (˜22%) conserved sequences, respectively. For the rest of the regulatory and auxillary proteins, a total of 15 conserved sequences, spanning from 12 to 38% of the individual protein length.

TABLE 4
Summary table for the number of highly conserved HIV-1
clade B sequences, their protein length in amino acids and
percentage coverage of total protein length.

| | | | Conserved | |
|---|---|---|---|---|
| | Protein length | | Sequence(s) length | % of protein |
| Protein | (aa)^{a} | Number^{b} | (aa)^{c} | length |
| Gag | 500 | 18 | 255 | 51 |
| Pol | 1,003 | 31 | 478 | 48 |
| Vif | 192 | 6 | 73 | 38 |
| Vpr | 78 | 2 | 24 | 31 |
| Tat | 86 | 2 | 26 | 30 |
| Rev | 116 | 1 | 15 | 13 |
| Vpu | 81 | 1 | 10 | 12 |
| Env | 856 | 14 | 190 | 22 |
| Nef | 206 | 3 | 30 | 15 |

^{a}Approximate size with respect to HXB2 sequences.
^{b}Total number of conserved sequences of 9 or more amino acids identified for each protein.
^{c}Total non-overlapping conserved sequence length.

TABLE 5
Highly conserved HIV-1 clade B sequences.
SEQ ID NOs: 55-132, in the order as shown.

| Protein | Position ^{a} | Sequences ^{b} |
|---|---|---|
| Gag | 1-11 | MGARASVLSGG |
| | 16-25 | WEKIRLRPGG |
| | 35-45 | VWASRELERFA |
| | 135-143 | SQNYPIVQN |
| | (129-137) | |
| | 154-164 | SPRTLNAWVKV |
| | (148-158) | |
| | 166-178 | EEKAFSPEVIPMF |
| | (160-172) | |
| | 180-208 | ALSEGATPQDLNTMLNTVGGHQAAMQMLK |
| | (174-202) | |
| | 210-220 | TINEEAAEWDR |
| | (204-214) | |
| | 231-253 | PGQMREPRGSDIAGTTSTLQEQI |
| | (225-247) | |
| | 259-269 | NPPIPVGEIYK |
| | (253-263) | |
| | 275-285 | GLNKIVRMYSP |
| | (269-279) | |
| | 293-315 | QGPKEPFRDYVDRFYKTLRAEQA |
| | (287-309) | |
| | 319-345 | VKNWMTETLLVQNANPDCKTILKALGP |
| | (313-339) | |
| | 347-362 | ATLEEMMTACQGVGGP |
| | (341-356) | |
| | 364-374 | HKARVLAEAMS |
| | (358-368) | |
| | 398-407 | KCFNCGKEGH |
| | (391-400) | |
| | 439-447 | NFLGKIWPS |
| | (432-440) | |
| | 449-457 | KGRPGNFLQ |
| | (442-450) | |
| Pol | 57-65 | PQITLWQRP |
| | 76-90 | KEALLDTGADDTVLE |
| | 100-109 | PKMIGGIGGF |
| | 103-112 | IGGIGGFIKV |
| | 150-174 | GCTLNFPISPIETVPVKLKPGMDGP |
| | 176-189 | VKQWPLTEEKIKAL |
| | 200-214 | GKISKIGPENPYNTP |
| | 226-237 | WRKLVDFRELNK |
| | 239-257 | TQDFWEVQLGIPHPAGLKK |
| | 259-272 | KSVTVLDVGDAYFS |
| | 279-289 | FRKYTAFTIPS |
| | 291-316 | NNETPGIRYQYNVLPQGWKGSPAIFQ |
| | 318-327 | SMTKILEPFR |
| | 340-350 | DDLYVGSDLEI |
| | 375-399 | KHQKEPPFLWMGYELHPDKWTVQPI |
| | 401-426 | LPEKDSWTVNDIQKLVGKLNWASQIY |
| | 453-471 | EAELELAENREILKEPVHG |
| | 716-724 | FLDGIDKAQ |
| | 750-758 | EIVASCDKC |
| | 755-764 | CDKCQLKGEA |
| | 766-786 | HGQVDCSPGIWQLDCTHLEGK |
| | 788-815 | ILVAVHVASGYIEAEVIPAETGQETAYF |
| | 817-826 | LKLAGRWPVK |
| | 841-850 | VKAACWWAGI |
| | 844-870 | ACWWAGIKQEFGIPYNPQSQGVVESMN |
| | 872-881 | ELKKIIGQVR |
| | 876-915 | IIGQVRDQAEHLKTAVQMAVFIHNFKRKGGIGGYSAGERI |
| | 934-944 | KIQNFRVYYRD |
| | 938-946 | FRVYYRDSR |
| | 948-970 | PLWKGPAKLLWKGEGAVVIQDNS |
| | 981-997 | KIIRDYGKQMAGDDCVA |
| Vif | 1-18 | MENRWQVMIVWQVDRMRI |
| | 52-60 | SSEVHIPLG |
| | 68-77 | TYWGLHTGER |
| | 79-90 | WHLGQGVSIEWR |
| | 138-150 | GHNKVGSLQYLAL |
| | 168-178 | KLTEDRWNKPQ |
| Vpr | 1-14 | MEQAPEDQGPQREP |
| | 18-27 | WTLELLEELK |
| Tat | 8-18 | LEPWKHPGSQP |
| | 43-57 | LGISYGRKKRRQRRR |
| Rev | 32-46 | EGTRQARRNRRRRWR |
| Vpu | 48-57 | ERAEDSGNES |
| Env | 33-58 | LWVTVYYGVPVWKEATTTLFCASDAK |
| | (34-59) | |
| | 65-76 | HNVWATHACVPT |
| | (66-77) | |
| | 114-128 | SLKPCVKLTPLCVTL |
| | (115-129) | |
| | 263-273 | NVSTVQCTHGI |
| | (241-251) | |
| | 275-289 | PVVSTQLLLNGSLAE |
| | (253-267) | |
| | 453-462 | VGKAMYAPPI |
| | (430-439) | |
| | 505-517 | DNWRSELYKYKVV |
| | (477-489) | |
| | 529-537 | AKRRVVQRE |
| | (501-509) | |
| | 548-563 | FLGFLGAAGSTMGAAS |
| | (519-534) | |
| | 573-581 | LLSGIVQQQ |
| | (544-552) | |
| | 595-611 | LQLTVWGIKQLQARVLA |
| | (566-582) | |
| | 618-635 | DQQLLGIWGCSGKLICTT |
| | (589-606) | |
| | 707-715 | WLWYIKIFI |
| | (678-686) | |
| | 848-857 | AIAVAEGTDR |
| | (819-828) | |
| Nef | 80-88 | PQVPLRPMT |
| | (72-80) | |
| | 129-140 | FPDWQNYTPGPG |
| | (121-132) | |
| | 147-155 | FGWCFKLVP |
| | (139-147) | |

^{a} Start and end alignment positions. Such positions corresponding to the HXB2 reference sequences are indicated in the brackets, only if they differ from the alignment positions. These differences are due to insertions and deletions in the protein alignment.
^{b} Sequences of 9 or more amino acids formed by one or by joining more than two contiguous nonamers that have primary nonamer incidence(s) of more than 80% and less than 10% representation of the primary variant. Sequences with less than 100 nonamers at that given nonamer position were ignored.

TABLE 6
Individual nonamers of the highly conserved HIV-1 clade B sequences and the HXB2
counterpart, those that are biclade and/or triclade conserved, those that are
HIV-1 specific and/or primate lentivirus group specific, and the incidences of
the highly conserved primary nonamers and their primary variants in the clade B
sequences ⁺. SEQ ID NOs: 133-636 for HXB2 nonamer in the order as shown and
637-1140 for the highly conserved primary nonamers of HIV-1 clade B in the order
as shown . . . SEQ ID NOs. of HIV-1 specific nonamers are 995 & 1029, while those
primate lentivirus group specific are: 637, 639-657, 661-743, 746-747, 756-759,
854-861, 863-866, 868-874, 876, 878-934, 940-994, 996-1028, 1030-1036, 1038-1052,
1054-1109, and 1113-1134. SEQ ID NOs. of biclade B and C highly conserved nonamers
are: 643-648, 651-677, 682-687, 696-704, 721-727, 735-737, 739-748, 750-782,
787-831, 834-853, 859-883, 885-902, 912-918, 920-923, 932-944, 952-963, 973-980,
983-995, 1015, 1022-1026, 1034-1035, 1041-1042, 1045-1046, 1055-1060, 1074-1095,
1098-1102, 1106-1116, 1129-1131, 1135-1139. SEQ ID NOs. of triclade A, B and C
highly conserved nonamers are: 645-648, 653, 671-677, 682-686, 696-704, 733-745,
1055-1060, 1073-1078, 1086, 1089-1094, 1098-1102, 1106-1112, 1116-1121, 1129-1131,
1135-1139.

| | | | | | | | Variants of the primary | | |
|---|---|---|---|---|---|---|---|---|---|
| | | | | | | | nonamers ^{f} | | |
| | | | | | | | | | Primary |
| | Nonamers analysed | | | HXB2 | | | Total | | variant |
| | | | Nonamer | Nonamer | Primary nonamer ^{e} | | Incidence | U- | inci- |
| Pro- | | | en- | Se- | Se- | Incidence | [No. | nique | dence ^{h} |
| tein | Position ^{a} | No. ^{b} | tropy ^{c} | quence ^{d} | quence ^{e} | [No. (%)] | (%)] ^{g} | [No.] | [No. (%)] |
| Gag | 1-9 \hat{ } | 1156 | 1.2 | MGARASVLS | ......... | 945 (~82) | 211 (~18) | 37 | 110 (~10) |
| | 2-10 | 1160 | 1.3 | GARASVLSG | ......... | 940 (~81) | 220 (~19) | 40 | 110 (~9) |
| | 3-11 \hat{ } | 1164 | 1.3 | ARASVLSGG | ......... | 942 (~81) | 222 (~19) | 42 | 108 (~9) |
| | 16-24 \hat{ } | 1551 | 1.1 | WEKIRLRPG | ......... | 1311 (~85) | 240 (~15) | 44 | 125 (~8) |
| | 17-25 \hat{ } | 1603 | 1.3 | EKIRLRPGG | ......... | 1320 (~82) | 283 (~18) | 55 | 124 (~8) |
| | 35-43 \hat{ } | 3196 | 0.9 | VWASRELER | ......... | 2890 (~90) | 306 (~10) | 62 | 78 (~2) |
| | 36-44 \hat{ }^{#} | 3269 | 0.8 | WASRELERF | ......... | 2981 (~91) | 288 (~9) | 57 | 80 (~2) |
| | 37-45 \hat{ }^{#} | 3316 | 1.0 | ASRELERFA | ......... | 2989 (~90) | 327 (~10) | 76 | 82 (~2) |
| | 135-143 | 3026 | 1.0 | SQNYPIVQN | ......... | 2684 (~89) | 342 (~11) | 64 | 79 (~3) |
| | (129-137) \hat{ }^{#$} | | | | | | | | |
| | 154-162 | 2049 | 0.5 | SPRTLNAWV | ......... | 1954 (~95) | 95 (~5) | 34 | 24 (~1) |
| | (148-156) \hat{ }^{#$} | | | | | | | | |
| | 155-163 | 2045 | 0.4 | PRTLNAWVK | ......... | 1977 (~97) | 68 (~3) | 31 | 10 (<1) |
| | (149-157) \hat{ }^{#$} | | | | | | | | |
| | 156-164 | 1976 | 0.4 | RTLNAWVKV | ......... | 1908 (~97) | 68 (~3) | 32 | 9 (<1) |
| | (150-158) \hat{ }^{#$} | | | | | | | | |
| | 166-174 | 1872 | 0.6 | EEKAFSPEV | ......... | 1768 (~94) | 104 (~6) | 47 | 10 (~1) |
| | (160-168) \hat{ } | | | | | | | | |
| | 167-175 | 1863 | 0.6 | EKAFSPEVI | ......... | 1760 (~94) | 103 (~6) | 50 | 10 (~1) |
| | (161-169) \hat{ } | | | | | | | | |
| | 168-176 | 1861 | 0.6 | KAFSPEVIP | ......... | 1760 (~95) | 101 (~5) | 46 | 11 (~1) |
| | (162-170) \hat{ }^{#} | | | | | | | | |
| | 169-177 | 1854 | 0.6 | AFSPEVIPM | ......... | 1767 (~95) | 87 (~5) | 45 | 11 (~1) |
| | (163-171) \hat{ }^{#} | | | | | | | | |
| | 170-178 | 1854 | 0.4 | FSPEVIPMF | ......... | 1782 (~96) | 72 (~4) | 35 | 12 (~1) |
| | (164-172) \hat{ }^{#$} | | | | | | | | |
| | 180-188 | 1922 | 0.9 | ALSEGATPQ | ......... | 1747 (~91) | 175 (~9) | 47 | 50 (~3) |
| | (174-182) \hat{ }^{#} | | | | | | | | |
| | 181-189 | 1923 | 0.9 | LSEGATPQD | ......... | 1750 (~91) | 173 (~9) | 46 | 50 (~3) |
| | (175-183) \hat{ }^{#} | | | | | | | | |
| | 182-190 | 1920 | 0.8 | SEGATPQDL | ......... | 1750 (~91) | 170 (~9) | 42 | 50 (~3) |
| | (176-184) \hat{ }^{#} | | | | | | | | |
| | 183-191 | 1920 | 0.7 | EGATPQDLN | ......... | 1798 (~94) | 122 (~6) | 44 | 20 (~1) |
| | (177-185) \hat{ }^{#} | | | | | | | | |
| | 184-192 | 1919 | 0.8 | GATPQDLNT | ......... | 1774 (~92) | 145 (~8) | 54 | 17 (~1) |
| | (178-186) ^{#} | | | | | | | | |
| | 185-193 | 1920 | 0.8 | ATPQDLNTM | ......... | 1775 (~92) | 145 (~8) | 57 | 17 (~1) |
| | (179-187) ^{#} | | | | | | | | |
| | 186-194 | 1922 | 0.8 | TPQDLNTML | ......... | 1777 (~92) | 145 (~8) | 58 | 17 (~1) |
| | (180-188) ^{#} | | | | | | | | |
| | 187-195 | 1920 | 0.8 | PQDLNTMLN | ......... | 1776 (~93) | 144 (~8) | 57 | 17 (~1) |
| | (181-189) \hat{ }^{#} | | | | | | | | |
| | 188-196 | 1921 | 0.9 | QDLNTMLNT | ......... | 1761 (~92) | 160 (~8) | 67 | 17 (~1) |
| | (182-190) \hat{ }^{#} | | | | | | | | |
| | 189-197 | 1922 | 0.7 | DLNTMLNTV | ......... | 1794 (~93) | 128 (~7) | 51 | 18 (~1) |
| | (183-191) \hat{ }^{#} | | | | | | | | |
| | 190-198 | 1921 | 0.7 | LNTMLNTVG | ......... | 1793 (~93) | 128 (~7) | 49 | 19 (~1) |
| | (184-192) \hat{ }^{#} | | | | | | | | |
| | 191-199 | 1918 | 0.7 | NTMLNTVGG | ......... | 1781 (~93) | 137 (~7) | 48 | 20 (~1) |
| | (185-193) \hat{ }^{#} | | | | | | | | |
| | 192-200 | 1922 | 0.8 | TMLNTVGGH | ......... | 1782 (~93) | 140 (~7) | 47 | 20 (~1) |
| | (186-194) \hat{ }^{#} | | | | | | | | |
| | 193-201 | 1913 | 0.6 | MLNTVGGHQ | ......... | 1797 (~94) | 116 (~6) | 36 | 24 (~1) |
| | (187-195) \hat{ }^{#} | | | | | | | | |
| | 194-202 | 1894 | 0.6 | LNTVGGHQA | ......... | 1786 (~94) | 108 (~6) | 33 | 24 (~1) |
| | (188-196) \hat{ }^{#} | | | | | | | | |
| | 195-203 | 1870 | 0.6 | NTVGGHQAA | ......... | 1763 (~94) | 107 (~6) | 33 | 26 (~1) |
| | (189-197) \hat{ }^{#} | | | | | | | | |
| | 196-204 | 1834 | 0.5 | TVGGHQAAM | ......... | 1732 (~94) | 102 (~6) | 30 | 26 (~1) |
| | (190 198) \hat{ }^{#} | | | | | | | | |
| | 197-205 | 1824 | 0.3 | VGGHQAAMQ | ......... | 1778 (~97) | 46 (~3) | 31 | 4 (<1) |
| | (191-199) \hat{ }^{#$} | | | | | | | | |
| | 198-206 | 1807 | 0.4 | GGHQAAMQM | ......... | 1733 (~96) | 74 (~4) | 29 | 34 (~2) |
| | (192-200) \hat{ }^{#$} | | | | | | | | |
| | 199-207 | 1798 | 0.4 | GHQAAMQML | ......... | 1727 (~96) | 71 (~4) | 26 | 34 (~2) |
| | (193-201) \hat{ }^{#$} | | | | | | | | |
| | 200-208 | 1795 | 0.4 | HQAAMQMLK | ......... | 1723 (~96) | 72 (~4) | 28 | 34 (~2) |
| | (194-202) \hat{ }^{#$} | | | | | | | | |
| | 210-218 | 1639 | 0.8 | TINEEAAEW | ......... | 1503 (~92) | 136 (~8) | 41 | 36 (~2) |
| | (204-212) \hat{ }^{#$} | | | | | | | | |
| | 211-219 | 1637 | 0.7 | INEEAAEWD | ......... | 1516 (~93) | 121 (~7) | 36 | 38 (~2) |
| | (205-213) \hat{ }^{#$} | | | | | | | | |
| | 212-220 | 1638 | 0.7 | NEEAAEWDR | ......... | 1518 (~93) | 120 (~7) | 39 | 37 (~2) |
| | (206-214) \hat{ }^{#$} | | | | | | | | |
| | 231-239 | 1566 | 1.1 | PGQMREPRG | ......... | 1344 (~86) | 222 (~14) | 40 | 83 (~5) |
| | (225-233) \hat{ } | | | | | | | | |
| | 232-240 | 1541 | 1.1 | GQMREPRGS | ......... | 1312 (~85) | 229 (~15) | 44 | 83 (~5) |
| | (226-234) \hat{ } | | | | | | | | |
| | 233-241 | 1541 | 1.2 | QMREPRGSD | ......... | 1311 (~85) | 230 (~15) | 42 | 84 (~5) |
| | (227-235) \hat{ } | | | | | | | | |
| | 234-242 | 1529 | 1.2 | MREPRGSDI | ......... | 1295 (~85) | 234 (~15) | 44 | 82 (~5) |
| | (228-236) \hat{ } | | | | | | | | |
| | 235-243 | 1532 | 0.7 | REPRGSDIA | ......... | 1421 (~93) | 111 (~7) | 35 | 46 (~3) |
| | (229-237) \hat{ }^{#$} | | | | | | | | |
| | 236-244 | 1531 | 0.6 | EPRGSDIAG | ......... | 1430 (~93) | 101 (~7) | 29 | 46 (~3) |
| | (230-238) \hat{ }^{#$} | | | | | | | | |
| | 237-245 | 1526 | 0.6 | PRGSDIAGT | ......... | 1446 (~95) | 80 (~5) | 32 | 15 (~1) |
| | (231-239) \hat{ }^{#$} | | | | | | | | |
| | 238-246 | 1525 | 0.6 | RGSDIAGTT | ......... | 1442 (~95) | 83 (~5) | 36 | 15 (~1) |
| | (232-240) \hat{ }^{#$} | | | | | | | | |
| | 239-247 | 1517 | 0.6 | GSDIAGTTS | ......... | 1438 (~95) | 79 (~5) | 33 | 15 (~1) |
| | (233-241) \hat{ }^{#$} | | | | | | | | |
| | 240-248 | 1515 | 1.1 | SDIAGTTST | ......... | 1278 (~84) | 237 (~16) | 34 | 143 (~9) |
| | (234-242) \hat{ }^{#} | | | | | | | | |
| | 241-249 | 1516 | 1.1 | DIAGTTSTL | ......... | 1281 (~84) | 235 (~16) | 33 | 143 (~9) |
| | (235-243) \hat{ } | | | | | | | | |
| | 242-250 | 1517 | 1.1 | IAGTTSTLQ | ......... | 1271 (~84) | 246 (~16) | 40 | 141 (~9) |
| | (236-244) \hat{ } | | | | | | | | |
| | 243-251 | 1514 | 1.2 | AGTTSTLQE | ......... | 1261 (~83) | 253 (~17) | 41 | 141 (~9) |
| | (237-245) \hat{ } | | | | | | | | |
| | 244-252 | 1511 | 1.2 | GTTSTLQEQ | ......... | 1261 (~83) | 250 (~17) | 41 | 141 (~9) |
| | (238-246) \hat{ } | | | | | | | | |
| | 245-253 | 1510 | 1.3 | TTSTLQEQI | ......... | 1235 (~82) | 275 (~18) | 44 | 140 (~9) |
| | (239-247) \hat{ } | | | | | | | | |
| | 259-267 | 1607 | 1.5 | NPPIPVGEI | ......... | 1297 (~81) | 310 (~19) | 52 | 86 (~5) |
| | (253-261) \hat{ } | | | | | | | | |
| | 260-268 | 1609 | 1.4 | PPIPVGEIY | ......... | 1303 (~81) | 306 (~19) | 52 | 87 (~5) |
| | (254-262) \hat{ } | | | | | | | | |
| | 261-269 | 1606 | 1.4 | PIPVGEIYK | ......... | 1302 (~81) | 304 (~19) | 49 | 87 (~5) |
| | (255-263) \hat{ } | | | | | | | | |
| | 275-283 | 1495 | 0.5 | GLNKIVRMY | ......... | 1425 (~95) | 70 (~5) | 26 | 19 (~1) |
| | (269-277) \hat{ }^{#$} | | | | | | | | |
| | 276-284 | 1453 | 0.6 | LNKIVRMYS | ......... | 1376 (~95) | 77 (~5) | 32 | 20 (~1) |
| | (270-278) \hat{ }^{#$} | | | | | | | | |
| | 277-285 | 1458 | 0.5 | NKIVRMYSP | ......... | 1384 (~95) | 74 (~5) | 31 | 20 (~1) |
| | (271-279) \hat{ }^{#$} | | | | | | | | |
| | 293-301 | 1462 | 0.5 | QGPKEPFRD | ......... | 1386 (~95) | 76 (~5) | 29 | 27 (~2) |
| | (287-295) \hat{ }^{#$} | | | | | | | | |
| | 294-302 | 1462 | 0.5 | GPKEPFRDY | ......... | 1388 (~95) | 74 (~5) | 28 | 27 (~2) |
| | (288-296) \hat{ }^{#$} | | | | | | | | |
| | 295-303 | 1461 | 0.5 | PKEPFRDYV | ......... | 1388 (~95) | 73 (~5) | 28 | 27 (~2) |
| | (289-297) \hat{ }^{#$} | | | | | | | | |
| | 296-304 | 1434 | 0.5 | KEPFRDYVD | ......... | 1363 (~95) | 71 (~5) | 28 | 26 (~2) |
| | (290-298) \hat{ }^{#$} | | | | | | | | |
| | 297-305 | 1433 | 0.5 | EPFRDYVDR | ......... | 1365 (~95) | 68 (~5) | 28 | 26 (~2) |
| | (291-299) \hat{ }^{#$} | | | | | | | | |
| | 298-306 | 1429 | 0.5 | PFRDYVDRF | ......... | 1355 (~95) | 74 (~5) | 27 | 27 (~2) |
| | (292-300) \hat{ }^{#$} | | | | | | | | |
| | 299-307 | 1428 | 0.8 | FRDYVDRFY | ......... | 1256 (~88) | 172 (~12) | 29 | 124 (~9) |
| | (293-301) \hat{ } | | | | | | | | |
| | 300-308 | 1374 | 0.9 | RDYVDRFYK | ......... | 1191 (~87) | 183 (~13) | 30 | 120 (~9) |
| | (294-302) \hat{ } | | | | | | | | |
| | 301-309 | 1372 | 1.1 | DYVDRFYKT | ......... | 1167 (~85) | 205 (~15) | 36 | 100 (~7) |
| | (295-303) \hat{ } | | | | | | | | |
| | 302-310 | 1372 | 1.1 | YVDRFYKTL | ......... | 1170 (~85) | 202 (~15) | 35 | 101 (~7) |
| | (296-304) \hat{ } | | | | | | | | |
| | 303-311 | 1313 | 1.0 | VDRFYKTLR | ......... | 1151 (~88) | 162 (~12) | 32 | 65 (~5) |
| | (297-305) \hat{ } | | | | | | | | |
| | 304-312 | 1309 | 1.0 | DRFYKTLRA | ......... | 1147 (~88) | 162 (~12) | 32 | 65 (~5) |
| | (298-306) \hat{ } | | | | | | | | |
| | 305-313 | 1303 | 1.0 | RFYKTLRAE | ......... | 1143 (~88) | 160 (~12) | 31 | 65 (~5) |
| | (299-307) \hat{ } | | | | | | | | |
| | 306-314 | 1289 | 1.0 | FYKTLRAEQ | ......... | 1128 (~88) | 161 (~12) | 30 | 65 (~5) |
| | (300-308) \hat{ } | | | | | | | | |
| | 307-315 | 1283 | 1.2 | YKTLRAEQA | ......... | 1084 (~84) | 199 (~16) | 36 | 57 (~4) |
| | (301-309) \hat{ } | | | | | | | | |
| | 319-327 | 1129 | 1.1 | VKNWMTETL | ......... | 992 (~88) | 137 (~12) | 36 | 41 (~4) |
| | (313-321) \hat{ } | | | | | | | | |
| | 320-328 | 1129 | 1.1 | KNWMTETLL | ......... | 987 (~87) | 142 (~13) | 37 | 41 (~4) |
| | (314-322) \hat{ } | | | | | | | | |
| | 321-329 | 1126 | 1.3 | NWMTETLLV | ......... | 969 (~86) | 157 (~14) | 44 | 40 (~4) |
| | (315-323) \hat{ } | | | | | | | | |
| | 322-330 | 1101 | 1.0 | WMTETLLVQ | ......... | 971 (~88) | 130 (~12) | 33 | 52 (~5) |
| | (316-324) \hat{ } | | | | | | | | |
| | 323-331 | 1101 | 1.0 | MTETLLVQN | ......... | 971 (~88) | 130 (~12) | 34 | 52 (~5) |
| | (317-325) \hat{ } | | | | | | | | |
| | 324-332 | 1101 | 1.3 | TETLLVQNA | ......... | 904 (~82) | 197 (~18) | 36 | 71 (~6) |
| | (318-326) \hat{ } | | | | | | | | |
| | 325-333 | 1101 | 1.4 | ETLLVQNAN | ......... | 898 (~82) | 203 (~18) | 38 | 70 (~6) |
| | (319-327) \hat{ } | | | | | | | | |
| | 326-334 | 1101 | 1.0 | TLLVQNANP | ......... | 963 (~87) | 138 (~13) | 34 | 70 (~6) |
| | (320-328) \hat{ }^{#} | | | | | | | | |
| | 327-335 | 1102 | 0.9 | LLVQNANPD | ......... | 970 (~88) | 132 (~12) | 31 | 71 (~6) |
| | (321-329) \hat{ }^{#} | | | | | | | | |
| | 328-336 | 1103 | 0.9 | LVQNANPDC | ......... | 970 (~88) | 133 (~12) | 32 | 71 (~6) |
| | (322-330) \hat{ }^{#} | | | | | | | | |
| | 329-337 | 1104 | 1.2 | VQNANPDCK | ......... | 910 (~82) | 194 (~18) | 32 | 70 (~6) |
| | (323-331) \hat{ }^{#} | | | | | | | | |
| | 330-338 | 1105 | 1.3 | QNANPDCKT | ......... | 904 (~82) | 201 (~18) | 33 | 71 (~6) |
| | (324-332) \hat{ }^{#} | | | | | | | | |
| | 331-339 | 1105 | 1.2 | NANPDCKTI | ......... | 909 (~82) | 196 (~18) | 36 | 70 (~6) |
| | (325-333) \hat{ }^{#} | | | | | | | | |
| | 332-340 | 1103 | 1.2 | ANPDCKTIL | ......... | 907 (~82) | 196 (~18) | 35 | 70 (~6) |
| | (326-334) \hat{ }^{#} | | | | | | | | |
| | 333-341 | 1103 | 1.0 | NPDCKTILK | ......... | 956 (~87) | 147 (~13) | 33 | 69 (~6) |
| | (327-335) \hat{ } | | | | | | | | |
| | 334-342 | 1103 | 0.9 | PDCKTILKA | ......... | 964 (~87) | 139 (~13) | 28 | 69 (~6) |
| | (328-336) \hat{ } | | | | | | | | |
| | 335-343 | 1106 | 1.0 | DCKTILKAL | ......... | 963 (~87) | 143 (~13) | 28 | 69 (~6) |
| | (329-337) \hat{ } | | | | | | | | |
| | 336-344 | 1107 | 1.0 | CKTILKALG | ......... | 963 (~87) | 144 (~13) | 29 | 69 (~6) |
| | (330-338) \hat{ } | | | | | | | | |
| | 337-345 | 1107 | 1.0 | KTILKALGP | ......... | 958 (~87) | 149 (~13) | 34 | 69 (~6) |
| | (331-339) \hat{ } | | | | | | | | |
| | 347-355 | 881 | 1.0 | ATLEEMMTA | ......... | 774 (~88) | 107 (~12) | 36 | 21 (~2) |
| | (341-349) \hat{ }^{$} | | | | | | | | |
| | 348-356 | 765 | 0.9 | TLEEMMTAC | ......... | 682 (~89) | 83 (~11) | 25 | 22 (~3) |
| | (342-350) \hat{ }^{$} | | | | | | | | |
| | 349-357 | 722 | 0.7 | LEEMMTACQ | ......... | 659 (~91) | 63 (~9) | 20 | 22 (~3) |
| | (343-351) \hat{ }^{#$} | | | | | | | | |
| | 350-358 | 706 | 0.6 | EEMMTACQG | ......... | 654 (~93) | 52 (~7) | 18 | 22 (~3) |
| | (344-352) \hat{ }^{#$} | | | | | | | | |
| | 351-359 | 698 | 0.7 | EMMTACQGV | ......... | 643 (~92) | 55 (~8) | 20 | 23 (~3) |
| | (345-353) \hat{ }^{#$} | | | | | | | | |
| | 352-360 | 709 | 0.7 | MMTACQGVG | ......... | 647 (~91) | 62 (~9) | 22 | 23 (~3) |
| | (346-354) \hat{ }^{$} | | | | | | | | |
| | 353-361 | 808 | 0.7 | MTACQGVGG | ......... | 742 (~92) | 66 (~8) | 23 | 22 (~3) |
| | (347-355) \hat{ }^{#$} | | | | | | | | |
| | 354-362 | 808 | 0.6 | TACQGVGGP | ......... | 744 (~92) | 64 (~8) | 20 | 35 (~4) |
| | (348-356) \hat{ }^{#$} | | | | | | | | |
| | 364-372 | 562 | 0.9 | HKARVLAEA | ......... | 478 (~85) | 84 (~15) | 16 | 46 (~8) |
| | (358-366) \hat{ }^{#$} | | | | | | | | |
| | 365-373 | 572 | 1.0 | KARVLAEAM | ......... | 485 (~85) | 87 (~15) | 17 | 48 (~8) |
| | (359-367) \hat{ }^{#$} | | | | | | | | |
| | 366-374 | 584 | 1.2 | ARVLAEAMS | ......... | 481 (~82) | 103 (~18) | 21 | 49 (~8) |
| | (360-368) \hat{ }^{#$} | | | | | | | | |
| | 398-406 | 794 | 1.1 | KCFNCGKEG | ......... | 686 (~86) | 108 (~14) | 33 | 28 (~4) |
| | (391-399) ^{#$} | | | | | | | | |
| | 399-407 | 792 | 1.1 | CFNCGKEGH | ......... | 686 (~87) | 106 (~13) | 34 | 28 (~4) |
| | (392-400) ^{#$} | | | | | | | | |
| | 439-447 | 875 | 0.7 | NFLGKIWPS | ......... | 810 (~93) | 65 (~7) | 20 | 20 (~2) |
| | (432-440) \hat{ }^{#} | | | | | | | | |
| | 449-457 | 885 | 0.9 | KGRPGNFLQ | ......... | 789 (~89) | 96 (~11) | 29 | 23 (~3) |
| | (442-450) \hat{ }^{#} | | | | | | | | |
| Pol | 57-65 ^{#} | 7010 | 0.4 | PQVTLWQRP | ..I...... | 6749 (~96) | 261 (~4) | 76 | 68 (~1) |
| | 76-84 | 11671 | 1.0 | KEALLDTGA | ......... | 10359 (~89) | 1312 | 105 | 299 (~3) |
| | | | | | | | (~11) | | |
| | 77-85 ^{#} | 11853 | 0.5 | EALLDTGAD | ......... | 11237 (~95) | 616 (~5) | 76 | 310 (~3) |
| | 78-86 ^{#} | 12120 | 0.6 | ALLDTGADD | ......... | 11249 (~93) | 871 (~7) | 76 | 314 (~3) |
| | 79-87 ^{#} | 12133 | 0.6 | LLDTGADDT | ......... | 11293 (~93) | 840 (~7) | 75 | 319 (~3) |
| | 80-88 ^{#} | 12149 | 0.7 | LDTGADDTV | ......... | 11149 (~92) | 1000 (~8) | 74 | 317 (~3) |
| | 81-89 ^{#} | 12074 | 0.9 | DTGADDTVL | ......... | 10726 (~89) | 1348 | 82 | 313 (~3) |
| | | | | | | | (~11) | | |
| | 82-90 ^{#} | 12069 | 1.0 | TGADDTVLE | ......... | 10656 (~88) | 1413 | 96 | 253 (~2) |
| | | | | | | | (~12) | | |
| | 100-108 \hat{ }^{#} | 11997 | 1.3 | PKMIGGIGG | ......... | 9670 (~81) | 2327 | 111 | 1080 (~9) |
| | | | | | | | (~19) | | |
| | 101-109 \hat{ }^{#} | 11925 | 1.4 | KMIGGIGGF | ......... | 9617 (~81) | 2308 | 137 | 987 (~8) |
| | | | | | | | (~19) | | |
| | 103-111 \hat{ }^{#} | 12013 | 1.2 | IGGIGGFIK | ......... | 10298 (~86) | 715 | 165 | 657 (~5) |
| | | | | | | | (~14) | | |
| | 104-112 \hat{ }^{#} | 12062 | 1.2 | GGIGGFIKV | ......... | 10366 (~86) | 1696 | 141 | 674 (~6) |
| | | | | | | | (~14) | | |
| | 150-158 ^{#} | 2737 | 0.8 | GCTLNFPIS | ......... | 2644 (~97) | 93 (~3) | 43 | 8 (<1) |
| | 151-159 ^{#} | 2737 | 0.8 | CTLNFPISP | ......... | 2625 (~96) | 112 (~4) | 42 | 16 (~1) |
| | 152-160 ^{#} | 2748 | 0.9 | TLNFPISPI | ......... | 2627 (~96) | 121 (~4) | 44 | 17 (~1) |
| | 153-161 ^{#} | 2761 | 1.2 | LNFPISPIE | ......... | 2533 (~92) | 228 (~8) | 54 | 60 (~2) |
| | 154-162 ^{#} | 2787 | 1.3 | NFPISPIET | ......... | 2530 (~91) | 257 (~9) | 62 | 60 (~2) |
| | 155-163 ^{#} | 2797 | 1.3 | FPISPIETV | ......... | 2536 (~91) | 261 (~9) | 62 | 55 (~2) |
| | 156-164 ^{#} | 7252 | 1.1 | PISPIETVP | ......... | 6523 (~90) | 729 (~10) | 97 | 216 (~3) |
| | 157-165 ^{#} | 7351 | 1.1 | ISPIETVPV | ......... | 6623 (~90) | 728 (~10) | 88 | 220 (~3) |
| | 158-166 ^{#} | 7220 | 1.1 | SPIETVPVK | ......... | 6458 (~89) | 762 (~11) | 96 | 243 (~3) |
| | 159-167 ^{#} | 7289 | 1.1 | PIETVPVKL | ......... | 6542 (~90) | 747 (~10) | 88 | 246 (~3) |
| | 160-168 ^{#} | 7091 | 1.1 | IETVPVKLK | ......... | 6392 (~90) | 699 (~10) | 86 | 251 (~4) |
| | 161-169 ^{#} | 7153 | 1.0 | ETVPVKLKP | ......... | 6470 (~90) | 683 (~10) | 85 | 261 (~4) |
| | 162-170 ^{#} | 7226 | 0.7 | TVPVKLKPG | ......... | 6886 (~95) | 340 (~5) | 65 | 63 (~1) |
| | 163-171 ^{#} | 7228 | 0.7 | VPVKLKPGM | ......... | 6887 (~95) | 341 (~5) | 73 | 64 (~1) |
| | 164-172 ^{#} | 7283 | 0.7 | PVKLKPGMD | ......... | 6956 (~96) | 327 (~4) | 68 | 67 (~1) |
| | 165-173 ^{#} | 7422 | 0.7 | VKLKPGMDG | ......... | 7098 (~96) | 324 (~4) | 74 | 68 (~1) |
| | 166-174 ^{#} | 7480 | 0.6 | KLKPGMDGP | ......... | 7169 (~96) | 311 (~4) | 66 | 72 (~1) |
| | 176-184 ^{#} | 8815 | 0.7 | VKQWPLTEE | ......... | 8340 (~95) | 475 (~5) | 70 | 123 (~1) |
| | 177-185 ^{#} | 8894 | 0.6 | KQWPLTEEK | ......... | 8530 (~96) | 364 (~4) | 64 | 62 (~1) |
| | 178-186 ^{#} | 8967 | 0.6 | QWPLTEEKI | ......... | 8608 (~96) | 359 (~4) | 60 | 60 (~1) |
| | 179-187 ^{#} | 9435 | 0.8 | WPLTEEKIK | ......... | 8842 (~94) | 593 (~6) | 78 | 95 (~1) |
| | 180-188 ^{#} | 9651 | 0.8 | PLTEEKIKA | ......... | 8987 (~93) | 664 (~7) | 83 | 141 (~1) |
| | 181-189 ^{#} | 9839 | 0.8 | LTEEKIKAL | ......... | 9155 (~93) | 684 (~7) | 86 | 139 (~1) |
| | 200-208 | 19898 | 1.1 | GKISKIGPE | ......... | 17048 (~86) | 2850 | 182 | 1064 (~5) |
| | | | | | | | (~14) | | |
| | 201-209 | 19919 | 1.1 | KISKIGPEN | ......... | 17080 (~86) | 2839 | 173 | 1063 (~5) |
| | | | | | | | (~14) | | |
| | 202-210 | 20000 | 1.1 | ISKIGPENP | ......... | 17207 (~86) | 2793 | 159 | 1066 (~5) |
| | | | | | | | (~14) | | |
| | 203-211 | 19977 | 1.1 | SKIGPENPY | ......... | 17194 (~86) | 2783 | 140 | 1070 (~5) |
| | | | | | | | (~14) | | |
| | 204-212 ^{#} | 19970 | 0.8 | KIGPENPYN | ......... | 18038 (~90) | 1932 | 125 | 1128 (~6) |
| | | | | | | | (~10) | | |
| | 205-213 ^{#} | 20230 | 0.4 | IGPENPYNT | ......... | 19407 (~96) | 823 (~4) | 106 | 233 (~1) |
| | 206-214 ^{#} | 20304 | 0.3 | GPENPYNTP | ......... | 19752 (~97) | 552 (~3) | 92 | 191 (~1) |
| | 226-234 ^{#} | 20066 | 1.0 | WRKLVDFRE | ......... | 17754 (~88) | 2312 | 112 | 857 (~4) |
| | | | | | | | (~12) | | |
| | 227-235 ^{#} | 20053 | 1.0 | RKLVDFREL | ......... | 17744 (~88) | 2309 | 111 | 855 (~4) |
| | | | | | | | (~12) | | |
| | 228-236 ^{#} | 20088 | 0.9 | KLVDFRELN | ......... | 17791 (~89) | 2297 | 112 | 860 (~4) |
| | | | | | | | (~11) | | |
| | 229-237 ^{#} | 20043 | 1.0 | LVDFRELNK | ......... | 17667 (~88) | 2376 | 116 | 850 (~4) |
| | | | | | | | (~12) | | |
| | 239-247 ^{#} | 20402 | 0.5 | TQDFWEVQL | ......... | 19299 (~95) | 1103 (~5) | 96 | 494 (~2) |
| | 240-248 ^{#} | 20415 | 0.5 | QDFWEVQLG | ......... | 19307 (~95) | 1108 (~5) | 93 | 494 (~2) |
| | 241-249 ^{#} | 20408 | 0.5 | DFWEVQLGI | ......... | 19276 (~94) | 1132 (~6) | 91 | 493 (~2) |
| | 242-250 ^{#} | 20454 | 0.4 | FWEVQLGIP | ......... | 19466 (~95) | 988 (~5) | 78 | 503 (~2) |
| | 243-251 ^{#} | 20430 | 0.5 | WEVQLGIPH | ......... | 19433 (~95) | 997 (~5) | 83 | 504 (~2) |
| | 244-252 ^{#} | 20484 | 0.3 | EVQLGIPHP | ......... | 19720 (~96) | 764 (~4) | 70 | 515 (~3) |
| | 245-253 ^{#} | 20263 | 0.8 | VQLGIPHPA | ......... | 17909 (~88) | 2354 | 94 | 1143 (~6) |
| | | | | | | | (~12) | | |
| | 246-254 ^{#} | 20407 | 0.7 | QLGIPHPAG | ......... | 18477 (~91) | 1930 (~9) | 91 | 1214 (~6) |
| | 247-255 ^{#} | 20312 | 0.8 | LGIPHPAGL | ......... | 18035 (~89) | 2277 | 96 | 1191 (~6) |
| | | | | | | | (~11) | | |
| | 248-256 ^{#} | 20018 | 1.2 | GIPHPAGLK | ......... | 16875 (~84) | 3143 | 124 | 1046 (~5) |
| | | | | | | | (~16) | | |
| | 249-257 ^{#} | 19699 | 1.6 | IPHPAGLKK | ......... | 15826 (~80) | 3873 | 175 | 926 (~5) |
| | | | | | | | (~20) | | |
| | 259-267 ^{#} | 19978 | 0.9 | KSVTVLDVG | ......... | 17949 (~90) | 2029 | 145 | 446 (~2) |
| | | | | | | | (~10) | | |
| | 260-268 ^{#} | 20006 | 0.8 | SVTVLDVGD | ......... | 18341 (~92) | 1665 (~8) | 139 | 452 (~2) |
| | 261-269 ^{#} | 19996 | 0.8 | VTVLDVGDA | ......... | 18371 (~92) | 1625 (~8) | 134 | 454 (~2) |
| | 262-270 ^{#} | 20126 | 0.6 | TVLDVGDAY | ......... | 18869 (~94) | 1257 (~6) | 116 | 454 (~2) |
| | 263-271 ^{#} | 20159 | 0.7 | VLDVGDAYF | ......... | 18778 (~93) | 1381 (~7) | 126 | 448 (~2) |
| | 264-272 ^{#} | 20336 | 0.5 | LDVGDAYFS | ......... | 19352 (~95) | 984 (~5) | 121 | 174 (~1) |
| | 279-287 ^{#} | 20343 | 0.2 | FRKYTAFTI | ......... | 20038 (~99) | 305 (~1) | 81 | 31 (<1) |
| | 280-288 ^{#} | 20352 | 0.2 | RKYTAFTIP | ......... | 20074 (~99) | 278 (~1) | 81 | 31 (<1) |
| | 281-289 ^{#} | 20183 | 0.2 | KYTAFTIPS | ......... | 19840 (~98) | 343 (~2) | 85 | 61 (<1) |
| | 291-299 ^{#} | 19417 | 1.2 | NNETPGIRY | ......... | 16532 (~85) | 2885 | 148 | 1286 (~7) |
| | | | | | | | (~15) | | |
| | 292-300 ^{#} | 19373 | 1.2 | NETPGIRYQ | ......... | 16479 (~85) | 2894 | 154 | 1285 (~7) |
| | | | | | | | (~15) | | |
| | 293-301 ^{#} | 19423 | 1.2 | ETPGIRYQY | ......... | 16566 (~85) | 2857 | 153 | 1297 (~7) |
| | | | | | | | (~15) | | |
| | 294-302 ^{#} | 19546 | 1.0 | TPGIRYQYN | ......... | 17030 (~87) | 2516 | 122 | 1359 (~7) |
| | | | | | | | (~13) | | |
| | 295-303 ^{#} | 19640 | 0.8 | PGIRYQYNV | ......... | 17372 (~88) | 2268 | 100 | 1388 (~7) |
| | | | | | | | (~12) | | |
| | 296-304 ^{#} | 19630 | 0.8 | GIRYQYNVL | ......... | 17352 (~88) | 2278 | 108 | 1386 (~7) |
| | | | | | | | (~12) | | |
| | 297-305 ^{#} | 19642 | 0.8 | IRYQYNVLP | ......... | 17362 (~88) | 2280 | 119 | 1386 (~7) |
| | | | | | | | (~12) | | |
| | 298-306 ^{#} | 20066 | 0.4 | RYQYNVLPQ | ......... | 19355 (~96) | 711 (~4) | 97 | 395 (~2) |
| | 299-307 ^{#} | 20112 | 0.4 | YQYNVLPQG | ......... | 19425 (~97) | 687 (~3) | 97 | 396 (~2) |
| | 300-308 ^{#} | 20116 | 0.3 | QYNVLPQGW | ......... | 19444 (~97) | 672 (~3) | 94 | 394 (~2) |
| | 301-309 ^{#} | 20187 | 0.3 | YNVLPQGWK | ......... | 19540 (~97) | 647 (~3) | 85 | 395 (~2) |
| | 302-310 ^{#} | 20220 | 0.3 | NVLPQGWKG | ......... | 19583 (~97) | 637 (~3) | 81 | 396 (~2) |
| | 303-311 ^{#} | 20275 | 0.3 | VLPQGWKGS | ......... | 19650 (~97) | 625 (~3) | 75 | 399 (~2) |
| | 304-312 ^{#} | 20299 | 0.3 | LPQGWKGSP | ......... | 19654 (~97) | 645 (~3) | 72 | 400 (~2) |
| | 305-313 ^{#} | 20222 | 0.5 | PQGWKGSPA | ......... | 19130 (~95) | 1092 (~5) | 74 | 449 (~2) |
| | 306-314 ^{#} | 20254 | 0.5 | QGWKGSPAI | ......... | 19131 (~94) | 1123 (~6) | 73 | 447 (~2) |
| | 307-315 ^{#} | 20287 | 0.3 | GWKGSPAIF | ......... | 19549 (~96) | 738 (~4) | 68 | 460 (~2) |
| | 308-316 ^{#} | 20314 | 0.4 | WKGSPAIFQ | ......... | 19554 (~96) | 760 (~4) | 75 | 460 (~2) |
| | 318-326 | 19881 | 1.2 | SMTKILEPF | ......... | 16450 (~83) | 3431 | 129 | 1279 (~6) |
| | | | | | | | (~17) | | |
| | 319-327 | 19823 | 1.3 | MTKILEPFR | ......... | 16309 (~82) | 3514 | 124 | 1266 (~6) |
| | | | | | | | (~18) | | |
| | 340-348 ^{#} | 20029 | 0.8 | DDLYVGSDL | ......... | 17989 (~90) | 2040 | 124 | 875 (~4) |
| | | | | | | | (~10) | | |
| | 341-349 ^{#} | 19982 | 0.9 | DLYVGSDLE | ......... | 17813 (~89) | 2169 | 136 | 855 (~4) |
| | | | | | | | (~11) | | |
| | 342-350 ^{#} | 19955 | 1.0 | LYVGSDLEI | ......... | 17610 (~88) | 2345 | 150 | 840 (~4) |
| | | | | | | | (~12) | | |
| | 375-383 ^{#} | 16175 | 1.2 | KHQKEPPFL | ......... | 13951 (~86) | 2224 | 180 | 701 (~4) |
| | | | | | | | (~14) | | |
| | 376-384 ^{#} | 15871 | 1.2 | HQKEPPFLW | ......... | 13650 (~86) | 2221 | 169 | 696 (~4) |
| | | | | | | | (~14) | | |
| | 377-385 ^{#} | 15330 | 1.1 | QKEPPFLWM | ......... | 13368 (~87) | 1962 | 161 | 707 (~5) |
| | | | | | | | (~13) | | |
| | 378-386 ^{#} | 14910 | 1.1 | KEPPFLWMG | ......... | 12992 (~87) | 1918 | 163 | 699 (~5) |
| | | | | | | | (~13) | | |
| | 379-387 ^{#} | 14938 | 1.0 | EPPFLWMGY | ......... | 13116 (~88) | 1822 | 138 | 810 (~5) |
| | | | | | | | (~12) | | |
| | 380-388 ^{#} | 13335 | 0.9 | PPFLWMGYE | ......... | 11782 (~88) | 1553 | 118 | 766 (~6) |
| | | | | | | | (~12) | | |
| | 381-389 ^{#} | 13201 | 0.9 | PFLWMGYEL | ......... | 11710 (~89) | 1491 | 108 | 769 (~6) |
| | | | | | | | (~11) | | |
| | 382-390 ^{#} | 12954 | 0.9 | FLWMGYELH | ......... | 11481 (~89) | 1473 | 108 | 766 (~6) |
| | | | | | | | (~11) | | |
| | 383-391 ^{#} | 12885 | 0.9 | LWMGYELHP | ......... | 11449 (~89) | 1436 | 104 | 789 (~6) |
| | | | | | | | (~11) | | |
| | 384-392 ^{#} | 12536 | 0.3 | WMGYELHPD | ......... | 12222 (~97) | 314 (~3) | 91 | 70 (~1) |
| | 385-393 ^{#} | 12412 | 0.5 | MGYELHPDK | ......... | 11937 (~96) | 475 (~4) | 91 | 100 (~1) |
| | 386-394 ^{#} | 12290 | 0.5 | GYELHPDKW | ......... | 11836 (~96) | 454 (~4) | 84 | 99 (~1) |
| | 387-395 ^{#} | 12105 | 0.5 | YELHPDKWT | ......... | 11630 (~96) | 475 (~4) | 84 | 99 (~1) |
| | 388-396 ^{#} | 11518 | 0.5 | ELHPDKWTV | ......... | 11047 (~96) | 471 (~4) | 92 | 98 (~1) |
| | 389-397 ^{#} | 11426 | 0.6 | LHPDKWTVQ | ......... | 10829 (~95) | 597 (~5) | 109 | 95 (~1) |
| | 390-398 ^{#} | 11226 | 0.7 | HPDKWTVQP | ......... | 10464 (~93) | 762 (~7) | 116 | 95 (~1) |
| | 391-399 ^{#} | 10726 | 0.8 | PDKWTVQPI | ......... | 9916 (~92) | 810 (~8) | 113 | 89 (~1) |
| | 401-409 \hat{ } | 4253 | 1.5 | LPEKDSWTV | ......... | 3466 (~81) | 787 (~19) | 103 | 212 (~5) |
| | 402-410 \hat{ } | 4111 | 1.4 | PEKDSWTVN | ......... | 3351 (~82) | 760 (~18) | 100 | 211 (~5) |
| | 403-411 \hat{ } | 3881 | 1.4 | EKDSWTVND | ......... | 3172 (~82) | 709 (~18) | 84 | 204 (~5) |
| | 404-412 \hat{ } | 3637 | 1.0 | KDSWTVNDI | ......... | 3162 (~87) | 475 (~13) | 53 | 257 (~7) |
| | 405-413 \hat{ } | 3614 | 0.9 | DSWTVNDIQ | ......... | 3179 (~88) | 435 (~12) | 48 | 257 (~7) |
| | 406-414 \hat{ }^{#} | 3601 | 0.5 | SWTVNDIQK | ......... | 3437 (~95) | 164 (~5) | 39 | 41 (~1) |
| | 407-415 \hat{ }^{#} | 3505 | 0.2 | WTVNDIQKL | ......... | 3441 (~98) | 64 (~2) | 28 | 24 (~1) |
| | 408-416 \hat{ }^{#} | 3483 | 0.2 | TVNDIQKLV | ......... | 3417 (~98) | 66 (~2) | 24 | 24 (~1) |
| | 409-417 ^{#} | 3474 | 0.2 | VNDIQKLVG | ......... | 3409 (~98) | 65 (~2) | 23 | 24 (~1) |
| | 410-418 \hat{ }^{#} | 3458 | 0.2 | NDIQKLVGK | ......... | 3393 (~98) | 65 (~2) | 22 | 24 (~1) |
| | 411-419 \hat{ }^{#} | 3448 | 0.2 | DIQKLVGKL | ......... | 3380 (~98) | 68 (~2) | 23 | 24 (~1) |
| | 412-420 \hat{ }^{#} | 3419 | 0.2 | IQKLVGKLN | ......... | 3349 (~98) | 70 (~2) | 26 | 24 (~1) |
| | 413-421 \hat{ }^{#} | 3428 | 0.2 | QKLVGKLNW | ......... | 3385 (~99) | 43 (~1) | 24 | 9 (<1) |
| | 414-422 ^{#} | 3387 | 0.2 | KLVGKLNWA | ......... | 3342 (~99) | 45 (~1) | 26 | 9 (<1) |
| | 415-423 \hat{ }^{#} | 3389 | 0.2 | LVGKLNWAS | ......... | 3339 (~99) | 50 (~1) | 27 | 10 (<1) |
| | 416-424 \hat{ }^{#} | 3372 | 0.2 | VGKLNWASQ | ......... | 3327 (~99) | 45 (~1) | 25 | 7 (<1) |
| | 417-425 \hat{ }^{#} | 3364 | 0.2 | GKLNWASQI | ......... | 3324 (~99) | 40 (~1) | 25 | 6 (<1) |
| | 418-426 \hat{ }^{#} | 3357 | 0.1 | KLNWASQIY | ......... | 3320 (~99) | 37 (~1) | 22 | 6 (<1) |
| | 453-461 \hat{ }^{#} | 431 | 0.7 | EAELELAEN | ......... | 391 (~91) | 40 (~9) | 13 | 25 (~6) |
| | 454-462 \hat{ }^{#} | 411 | 0.7 | AELELAENR | ......... | 372 (~91) | 39 (~9) | 13 | 25 (~6) |
| | 455-463 \hat{ }^{#} | 401 | 0.6 | ELELAENRE | ......... | 364 (~91) | 37 (~9) | 12 | 25 (~6) |
| | 456-464 ^{#} | 400 | 0.7 | LELAENREI | ......... | 361 (~90) | 39 (~10) | 15 | 23 (~6) |
| | 457-465 \hat{ }^{#} | 396 | 0.7 | ELAENREIL | ......... | 357 (~90) | 39 (~10) | 14 | 23 (~6) |
| | 458-466 ^{#} | 390 | 1.1 | LAENREILK | ......... | 327 (~84) | 63 (~16) | 17 | 23 (~6) |
| | 459-467 \hat{ }^{#} | 342 | 1.4 | AENREILKE | ......... | 277 (~81) | 65 (~19) | 22 | 19 (~6) |
| | 460-468 \hat{ }^{#} | 337 | 1.2 | ENREILKEP | ......... | 284 (~84) | 53 (~16) | 25 | 16 (~5) |
| | 461-469 \hat{ }^{#} | 331 | 1.2 | NREILKEPV | ......... | 279 (~84) | 52 (~16) | 26 | 16 (~5) |
| | 462-470 \hat{ }^{#} | 312 | 1.3 | REILKEPVH | ......... | 261 (~84) | 51 (~16) | 26 | 15 (~5) |
| | 463-471 \hat{ }^{#} | 310 | 1.3 | EILKEPVHG | ......... | 261 (~84) | 49 (~16) | 25 | 15 (~5) |
| | 716-724 \hat{ }^{#} | 523 | 1.0 | FLDGIDKAQ | ......... | 448 (~86) | 75 (~14) | 17 | 21 (~4) |
| | 750-758 \hat{ } | 545 | 1.3 | EIVASCDKC | ......... | 436 (~80) | 109 (~20) | 16 | 31 (~6) |
| | 755-763 \hat{ }^{#} | 553 | 1.4 | CDKCQLKGE | ......... | 448 (~81) | 105 (~19) | 21 | 21 (~4) |
| | 756-764 \hat{ }^{#} | 553 | 1.4 | DKCQLKGEA | ......... | 449 (~81) | 104 (~19) | 22 | 21 (~4) |
| | 766-774 \hat{ }^{#} | 559 | 0.6 | HGQVDCSPG | ......... | 518 (~93) | 41 (~7) | 13 | 20 (~4) |
| | 767-775 \hat{ }^{#} | 558 | 0.6 | GQVDCSPGI | ......... | 517 (~93) | 41 (~7) | 15 | 16 (~3) |
| | 768-776 \hat{ }^{#} | 559 | 0.6 | QVDCSPGIW | ......... | 517 (~92) | 42 (~8) | 15 | 16 (~3) |
| | 769-777 \hat{ }^{#} | 557 | 0.6 | VDCSPGIWQ | ......... | 514 (~92) | 43 (~8) | 16 | 16 (~3) |
| | 770-778 \hat{ }^{#} | 557 | 0.6 | DCSPGIWQL | ......... | 516 (~93) | 41 (~7) | 17 | 10 (~2) |
| | 771-779 \hat{ }^{#} | 557 | 0.6 | CSPGIWQLD | ......... | 516 (~93) | 41 (~7) | 16 | 10 (~2) |
| | 772-780 \hat{ }^{#} | 557 | 0.6 | SPGIWQLDC | ......... | 517 (~93) | 40 (~7) | 14 | 10 (~2) |
| | 773-781 \hat{ }^{#} | 559 | 0.5 | PGIWQLDCT | ......... | 523 (~94) | 36 (~6) | 12 | 10 (~2) |
| | 774-782 \hat{ }^{#} | 559 | 0.5 | GIWQLDCTH | ......... | 523 (~94) | 36 (~6) | 12 | 10 (~2) |
| | 775-783 \hat{ }^{#} | 558 | 0.6 | IWQLDCTHL | ......... | 522 (~94) | 36 (~6) | 16 | 8 (~1) |
| | 776-784 \hat{ }^{#} | 557 | 0.5 | WQLDCTHLE | ......... | 524 (~94) | 33 (~6) | 13 | 14 (~3) |
| | 777-785 \hat{ }^{#} | 557 | 0.6 | QLDCTHLEG | ......... | 521 (~94) | 36 (~6) | 15 | 14 (~3) |
| | 778-786 \hat{ }^{#} | 558 | 0.6 | LDCTHLEGK | ......... | 519 (~93) | 39 (~7) | 16 | 14 (~3) |
| | 788-796 \hat{ }^{#} | 564 | 0.7 | ILVAVHVAS | ......... | 506 (~90) | 58 (~10) | 13 | 38 (~7) |
| | 789-797 \hat{ }^{#} | 560 | 0.7 | LVAVHVASG | ......... | 502 (~90) | 58 (~10) | 15 | 38 (~7) |
| | 790-798 \hat{ }^{#} | 560 | 0.6 | VAVHVASGY | ......... | 517 (~92) | 43 (~8) | 12 | 27 (~5) |
| | 791-799 \hat{ } | 556 | 0.9 | AVHVASGYI | ......... | 485 (~87) | 71 (~13) | 15 | 27 (~5) |
| | 792-800 \hat{ } | 556 | 0.9 | VHVASGYIE | ......... | 485 (~87) | 71 (~13) | 15 | 27 (~5) |
| | 793-801 \hat{ } | 557 | 0.9 | HVASGYIEA | ......... | 488 (~88) | 69 (~12) | 15 | 27 (~5) |
| | 794-802 \hat{ } | 557 | 0.9 | VASGYIEAE | ......... | 487 (~87) | 70 (~13) | 16 | 27 (~5) |
| | 795-803 \hat{ } | 554 | 0.9 | ASGYIEAEV | ......... | 487 (~88) | 67 (~12) | 14 | 27 (~5) |
| | 796-804 \hat{ } | 554 | 0.9 | SGYIEAEVI | ......... | 487 (~88) | 67 (~12) | 15 | 27 (~5) |
| | 797-805 \hat{ } | 554 | 0.9 | GYIEAEVIP | ......... | 482 (~87) | 72 (~13) | 17 | 27 (~5) |
| | 798-806 \hat{ } | 557 | 1.0 | YIEAEVIPA | ......... | 480 (~86) | 77 (~14) | 20 | 25 (~4) |
| | 799-807 \hat{ } | 560 | 0.8 | IEAEVIPAE | ......... | 506 (~90) | 54 (~10) | 20 | 12 (~2) |
| | 800-808 \hat{ }^{#} | 564 | 0.5 | EAEVIPAET | ......... | 532 (~94) | 32 (~6) | 18 | 6 (~1) |
| | 801-809 \hat{ }^{#} | 564 | 0.5 | AEVIPAETG | ......... | 531 (~94) | 33 (~6) | 18 | 6 (~1) |
| | 802-810 \hat{ }^{#} | 564 | 0.6 | EVIPAETGQ | ......... | 531 (~94) | 33 (~6) | 19 | 6 (~1) |
| | 803-811 \hat{ }^{#} | 563 | 0.5 | VIPAETGQE | ......... | 531 (~94) | 32 (~6) | 19 | 5 (~1) |
| | 804-812 \hat{ }^{#} | 584 | 0.7 | IPAETGQET | ......... | 544 (~93) | 40 (~7) | 24 | 5 (~1) |
| | 805-813 \hat{ }^{#} | 585 | 0.6 | PAETGQETA | ......... | 546 (~93) | 39 (~7) | 23 | 5 (~1) |
| | 806-814 \hat{ }^{#} | 584 | 0.6 | AETGQETAY | ......... | 550 (~94) | 34 (~6) | 21 | 5 (~1) |
| | 807-815 \hat{ } | 584 | 0.5 | ETGQETAYF | ......... | 551 (~94) | 33 (~6) | 15 | 9 (~2) |
| | 817-825 \hat{ }^{#} | 583 | 0.6 | LKLAGRWPV | ......... | 528 (~91) | 55 (~9) | 9 | 41 (~7) |
| | 818-826 \hat{ }^{#} | 575 | 1.1 | KLAGRWPVK | ......... | 475 (~83) | 100 (~17) | 14 | 38 (~7) |
| | 841-849 \hat{ }^{#} | 587 | 1.0 | VRAACWWAG | .K....... | 512 (~87) | 75 (~13) | 22 | 16 (~3) |
| | 842-850 \hat{ }^{#} | 588 | 1.1 | RAACWWAGI | K........ | 494 (~84) | 94 (~16) | 19 | 35 (~6) |
| | 844-852 \hat{ } | 585 | 1.3 | ACWWAGIKQ | ......... | 482 (~82) | 103 (~18) | 26 | 36 (~6) |
| | 845-853 \hat{ } | 585 | 1.3 | CWWAGIKQE | ......... | 480 (~82) | 105 (~18) | 27 | 36 (~6) |
| | 846-854 \hat{ } | 585 | 1.3 | WWAGIKQEF | ......... | 480 (~82) | 105 (~18) | 27 | 36 (~6) |
| | 847-855 \hat{ } | 584 | 1.3 | WAGIKQEFG | ......... | 478 (~82) | 106 (~18) | 28 | 36 (~6) |
| | 848-856 \hat{ } | 582 | 1.3 | AGIKQEFGI | ......... | 476 (~82) | 106 (~18) | 28 | 36 (~6) |
| | 849-857 \hat{ } | 582 | 1.3 | GIKQEFGIP | ......... | 477 (~82) | 105 (~18) | 25 | 36 (~6) |
| | 850-858 \hat{ } | 582 | 1.2 | IKQEFGIPY | ......... | 480 (~82) | 102 (~18) | 19 | 36 (~6) |
| | 851-859 \hat{ } | 580 | 0.8 | KQEFGIPYN | ......... | 516 (~89) | 64 (~11) | 16 | 21 (~4) |
| | 852-860 \hat{ }^{#} | 583 | 0.5 | QEFGIPYNP | ......... | 547 (~94) | 36 (~6) | 10 | 23 (~4) |
| | 853-861 \hat{ }^{#} | 582 | 0.2 | EFGIPYNPQ | ......... | 569 (~98) | 13 (~2) | 9 | 3 (~1) |
| | 854-862 \hat{ }^{#} | 582 | 0.2 | FGIPYNPQS | ......... | 572 (~98) | 10 (~2) | 8 | 2 (<11) |
| | 855-863 ^{#} | 582 | 0.2 | GIPYNPQSQ | ......... | 571 (~98) | 11 (~2) | 10 | 2 (<11) |
| | 856-864 ^{#} | 583 | 0.2 | IPYNPQSQG | ......... | 573 (~98) | 10 (~2) | 9 | 2 (<11) |
| | 857-865 ^{#} | 584 | 0.2 | PYNPQSQGV | ......... | 574 (~98) | 10 (~2) | 8 | 3 (~1) |
| | 858-866 ^{#} | 578 | 0.4 | YNPQSQGVV | ......... | 544 (~94) | 34 (~6) | 10 | 23 (~4) |
| | 859-867 ^{#} | 579 | 0.5 | NPQSQGVVE | ......... | 540 (~93) | 39 (~7) | 11 | 23 (~4) |
| | 860-868 \hat{ }^{#} | 581 | 0.8 | PQSQGVVES | ......... | 517 (~89) | 64 (~11) | 12 | 25 (~4) |
| | 861-869 \hat{ }^{#} | 579 | 1.2 | QSQGVVESM | ......... | 474 (~82) | 105 (~18) | 16 | 25 (~4) |
| | 862-870 \hat{ }^{#} | 579 | 1.2 | SQGVVESMN | ......... | 474 (~82) | 105 (~18) | 16 | 25 (~4) |
| | 872-880 \hat{ }^{#} | 583 | 1.4 | ELKKIIGQV | ......... | 472 (~81) | 111 (~19) | 31 | 25 (~4) |
| | 873-881 \hat{ }^{#} | 583 | 1.4 | LKKIIGQVR | ......... | 475 (~81) | 108 (~19) | 30 | 25 (~4) |
| | 876-884 \hat{ } | 579 | 1.3 | IIGQVRDQA | ......... | 470 (~81) | 109 (~19) | 25 | 26 (~4) |
| | 877-885 \hat{ } | 578 | 1.3 | IGQVRDQAE | ......... | 471 (~81) | 107 (~19) | 23 | 26 (~4) |
| | 878-886 \hat{ } | 578 | 1.3 | GQVRDQAEH | ......... | 471 (~81) | 107 (~19) | 23 | 26 (~4) |
| | 879-887 \hat{ } | 578 | 0.7 | QVRDQAEHL | ......... | 523 (~90) | 55 (~10) | 15 | 28 (~5) |
| | 880-888 \hat{ } | 577 | 0.9 | VRDQAEHLK | ......... | 502 (~87) | 75 (~13) | 18 | 27 (~5) |
| | 881-889 \hat{ } | 577 | 0.6 | RDQAEHLKT | ......... | 530 (~92) | 47 (~8) | 16 | 21 (~4) |
| | 882-890 \hat{ } | 577 | 0.7 | DQAEHLKTA | ......... | 529 (~92) | 48 (~8) | 17 | 21 (~4) |
| | 883-891 \hat{ }^{#} | 578 | 0.6 | QAEHLKTAV | ......... | 536 (~93) | 42 (~7) | 17 | 20 (~3) |
| | 884-892 \hat{ }^{#} | 577 | 0.6 | AEHLKTAVQ | ......... | 537 (~93) | 40 (~7) | 16 | 20 (~3) |
| | 885-893 \hat{ }^{#} | 579 | 0.6 | EHLKTAVQM | ......... | 539 (~93) | 40 (~7) | 15 | 20 (~3) |
| | 886-894 \hat{ }^{#} | 580 | 0.6 | HLKTAVQMA | ......... | 540 (~93) | 40 (~7) | 15 | 20 (~3) |
| | 887-895 \hat{ }^{#} | 582 | 0.5 | LKTAVQMAV | ......... | 547 (~94) | 35 (~6) | 13 | 20 (~3) |
| | 888-896 \hat{ }^{#} | 582 | 0.7 | KTAVQMAVF | ......... | 527 (~91) | 55 (~9) | 13 | 23 (~4) |
| | 889-897 \hat{ }^{#} | 582 | 0.7 | TAVQMAVFI | ......... | 525 (~90) | 57 (~10) | 12 | 23 (~4) |
| | 890-898 \hat{ }^{#} | 585 | 0.7 | AVQMAVFIH | ......... | 526 (~90) | 59 (~10) | 13 | 23 (~4) |
| | 891-899 \hat{ }^{#} | 566 | 0.7 | VQMAVFIHN | ......... | 512 (~90) | 54 (~10) | 11 | 22 (~4) |
| | 892-900 \hat{ }^{#} | 565 | 0.6 | QMAVFIHNF | ......... | 513 (~91) | 52 (~9) | 10 | 21 (~4) |
| | 893-901 \hat{ }^{#} | 566 | 0.6 | MAVFIHNFK | ......... | 514 (~91) | 52 (~9) | 10 | 21 (~4) |
| | 894-902 \hat{ }^{#} | 566 | 0.7 | AVFIHNFKR | ......... | 509 (~90) | 57 (~10) | 13 | 20 (~4) |
| | 895-903 \hat{ } | 561 | 0.9 | VFIHNFKRK | ......... | 492 (~88) | 69 (~12) | 13 | 20 (~4) |
| | 896-904 \hat{ } | 561 | 0.9 | FIHNFKRKG | ......... | 491 (~88) | 70 (~12) | 15 | 20 (~4) |
| | 897-905 \hat{ } | 562 | 0.7 | IHNFKRKGG | ......... | 513 (~91) | 49 (~9) | 11 | 20 (~4) |
| | 898-906 \hat{ } | 559 | 0.4 | HNFKRKGGI | ......... | 531 (~95) | 28 (~5) | 9 | 10 (~2) |
| | 899-907 \hat{ } | 557 | 0.4 | NFKRKGGIG | ......... | 532 (~96) | 25 (~4) | 8 | 9 (~2) |
| | 900-908 \hat{ } | 557 | 0.9 | FKRKGGIGG | ......... | 491 (~88) | 66 (~12) | 14 | 23 (~4) |
| | 901-909 \hat{ } | 557 | 0.9 | KRKGGIGGY | ......... | 495 (~89) | 62 (~11) | 13 | 23 (~4) |
| | 902-910 \hat{ } | 557 | 1.2 | RKGGIGGYS | ......... | 460 (~83) | 97 (~17) | 18 | 33 (~6) |
| | 903-911 \hat{ } | 558 | 1.1 | KGGIGGYSA | ......... | 466 (~84) | 92 (~16) | 15 | 33 (~6) |
| | 904-912 \hat{ }^{#} | 562 | 1.0 | GGIGGYSAG | ......... | 476 (~85) | 86 (~15) | 14 | 33 (~6) |
| | 905-913 \hat{ }^{#} | 560 | 1.0 | GIGGYSAGE | ......... | 475 (~85) | 85 (~15) | 15 | 32 (~6) |
| | 906-914 \hat{ }^{#} | 560 | 1.0 | IGGYSAGER | ......... | 476 (~85) | 84 (~15) | 14 | 32 (~6) |
| | 907-915 \hat{ }^{#} | 559 | 1.1 | GGYSAGERI | ......... | 474 (~85) | 85 (~15) | 16 | 24 (~4) |
| | 934-942 \hat{ }^{#} | 545 | 1.4 | KIQNFRVYY | ......... | 454 (~83) | 91 (~17) | 34 | 13 (~2) |
| | 935-943 \hat{ }^{#} | 546 | 1.3 | IQNFRVYYR | ......... | 459 (~84) | 87 (~16) | 27 | 19 (~3) |
| | 936-944 \hat{ }^{#} | 549 | 0.9 | QNFRVYYRD | ......... | 484 (~88) | 65 (~12) | 19 | 19 (~3) |
| | 938-946 \hat{ }^{#} | 547 | 1.2 | FRVYYRDSR | ......... | 439 (~80) | 108 (~20) | 13 | 48 (~9) |
| | 948-956 \hat{ } | 543 | 1.1 | PLWKGPAKL | ......... | 449 (~83) | 94 (~17) | 11 | 42 (~8) |
| | 949-957 \hat{ } | 543 | 1.1 | LWKGPAKLL | ......... | 449 (~83) | 94 (~17) | 11 | 42 (~8) |
| | 950-958 \hat{ }^{#} | 542 | 0.4 | WKGPAKLLW | ......... | 514 (~95) | 28 (~5) | 7 | 21 (~4) |
| | 951-959 \hat{ }^{#} | 542 | 0.4 | KGPAKLLWK | ......... | 515 (~95) | 27 (~5) | 6 | 21 (~4) |
| | 952-960 \hat{ }^{#} | 543 | 0.3 | GPAKLLWKG | ......... | 517 (~95) | 26 (~5) | 5 | 21 (~4) |
| | 953-961 \hat{ }^{#} | 541 | 0.3 | PAKLLWKGE | ......... | 517 (~96) | 24 (~4) | 3 | 21 (~4) |
| | 954-962 \hat{ }^{#} | 542 | 0.3 | AKLLWKGEG | ......... | 518 (~96) | 24 (~4) | 3 | 21 (~4) |
| | 955-963 \hat{ }^{#} | 542 | 0.3 | KLLWKGEGA | ......... | 518 (~96) | 24 (~4) | 3 | 21 (~4) |
| | 956-964 \hat{ }^{#} | 541 | 0.0 | LLWKGEGAV | ......... | 541 (~100) | 0 (0) | 0 | 0 (0) |
| | 957-965 \hat{ }^{#} | 544 | 0.0 | LWKGEGAVV | ......... | 544 (~100) | 0 (0) | 0 | 0 (0) |
| | 958-966 \hat{ }^{#} | 539 | 0.1 | WKGEGAVVI | ......... | 531 (~99) | 8 (~1) | 2 | 6 (~1) |
| | 959-967 \hat{ }^{#} | 538 | 0.2 | KGEGAVVIQ | ......... | 527 (~98) | 11 (~2) | 5 | 6 (~1) |
| | 960-968 \hat{ }^{#} | 538 | 0.4 | GEGAVVIQD | ......... | 517 (~96) | 21 (~4) | 7 | 6 (~1) |
| | 961-969 \hat{ }^{#} | 532 | 0.7 | EGAVVIQDN | ......... | 481 (~90) | 51 (~10) | 12 | 25 (~5) |
| | 962-970 *^{#} | 531 | 0.9 | GAVVIQDNS | ......... | 471 (~89) | 60 (~11) | 18 | 23 (~4) |
| | 981-989 \hat{ } | 516 | 0.5 | KIIRDYGKQ | ......... | 484 (~94) | 32 (~6) | 10 | 10 (~2) |
| | 982-990 \hat{ } | 515 | 0.6 | IIRDYGKQM | ......... | 480 (~93) | 35 (~7) | 10 | 10 (~2) |
| | 983-991 \hat{ } | 516 | 0.6 | IRDYGKQMA | ......... | 481 (~93) | 35 (~7) | 10 | 10 (~2) |
| | 984-992 \hat{ } | 514 | 0.4 | RDYGKQMAG | ......... | 488 (~95) | 26 (~5) | 8 | 10 (~2) |
| | 985-993 \hat{ } | 511 | 0.7 | DYGKQMAGD | ......... | 466 (~91) | 45 (~9) | 15 | 15 (~3) |
| | 986-994 \hat{ } | 506 | 0.8 | YGKQMAGDD | ......... | 457 (~90) | 49 (~10) | 14 | 19 (~4) |
| | 987-995 \hat{ } | 507 | 0.8 | GKQMAGDDC | ......... | 456 (~90) | 51 (~10) | 20 | 14 (~3) |
| | 988-996 \hat{ } | 505 | 0.9 | KQMAGDDCV | ......... | 452 (~90) | 53 (~10) | 23 | 13 (~3) |
| | 989-997 \hat{ } | 505 | 0.8 | QMAGDDCVA | ......... | 456 (~90) | 49 (~10) | 20 | 13 (~3) |
| Vif | 1-9 \hat{ } | 1140 | 0.6 | MENRWQVMI | ......... | 1069 (~94) | 71 (~6) | 24 | 19 (~2) |
| | 2-10 \hat{ } | 1140 | 0.6 | ENRWQVMIV | ......... | 1066 (~94) | 74 (~6) | 25 | 19 (~2) |
| | 3-11 \hat{ } | 1141 | 0.6 | NRWQVMIVW | ......... | 1067 (~94) | 74 (~6) | 24 | 19 (~2) |
| | 4-12 \hat{ } | 1141 | 0.6 | RWQVMIVWQ | ......... | 1069 (~94) | 72 (~6) | 23 | 19 (~2) |
| | 5-13 \hat{ } | 1141 | 0.6 | WQVMIVWQV | ......... | 1068 (~94) | 73 (~6) | 23 | 19 (~2) |
| | 6-14 \hat{ } | 1141 | 0.7 | QVMIVWQVD | ......... | 1058 (~93) | 83 (~7) | 26 | 19 (~2) |
| | 7-15 \hat{ } | 1141 | 0.7 | VMIVWQVDR | ......... | 1057 (~93) | 84 (~7) | 28 | 19 (~2) |
| | 8-16 \hat{ } | 1142 | 0.7 | MIVWQVDRM | ......... | 1069 (~94) | 73 (~6) | 31 | 13 (~1) |
| | 9-17 \hat{ } | 1143 | 0.6 | IVWQVDRMR | ......... | 1070 (~94) | 73 (~6) | 29 | 13 (~1) |
| | 10-18 \hat{ } | 1143 | 0.6 | VWQVDRMRI | ......... | 1081 (~95) | 62 (~5) | 29 | 9 (~1) |
| | 52-60 \hat{ }^{#} | 1141 | 1.5 | SSEVHIPLG | ......... | 947 (~83) | 194 (~17) | 46 | 42 (~4) |
| | 68-76 \hat{ } | 1138 | 0.9 | TYWGLHTGE | ......... | 1010 (~89) | 128 (~11) | 30 | 41 (~4) |
| | 69-77 \hat{ } | 1138 | 1.2 | YWGLHTGER | ......... | 962 (~85) | 176 (~15) | 32 | 43 (~4) |
| | 79-87 \hat{ } | 1140 | 1.1 | WHLGQGVSI | ......... | 993 (~87) | 147 (~13) | 29 | 33 (~3) |
| | 80-88 \hat{ } | 1140 | 1.1 | HLGQGVSIE | ......... | 988 (~87) | 152 (~13) | 32 | 32 (~3) |
| | 81-89 \hat{ } | 1141 | 1.1 | LGQGVSIEW | ......... | 989 (~87) | 152 (~13) | 31 | 35 (~3) |
| | 82-90 \hat{ } | 1142 | 1.2 | GQGVSIEWR | ......... | 973 (~85) | 169 (~15) | 33 | 35 (~3) |
| | 138-146 \hat{ }^{#} | 1134 | 1.6 | GHNKVGSLQ | ......... | 916 (~81) | 218 (~19) | 43 | 41 (~4) |
| | 139-147 \hat{ }^{#} | 1133 | 1.6 | HNKVGSLQY | ......... | 912 (~80) | 221 (~20) | 45 | 41 (~4) |
| | 140-148 \hat{ }^{#} | 1132 | 1.6 | NKVGSLQYL | ......... | 913 (~81) | 219 (~19) | 43 | 41 (~4) |
| | 141-149 \hat{ }^{#} | 1134 | 1.1 | KVGSLQYLA | ......... | 977 (~86) | 157 (~14) | 29 | 46 (~4) |
| | 142-150 \hat{ }^{#} | 1137 | 0.7 | VGSLQYLAL | ......... | 1058 (~93) | 79 (~7) | 26 | 18 (~2) |
| | 168-176 \hat{ } | 1135 | 0.8 | KLTEDRWNK | ......... | 1019 (~90) | 116 (~10) | 25 | 64 (~6) |
| | 169-177 \hat{ } | 1135 | 0.9 | LTEDRWNKP | ......... | 1000 (~88) | 135 (~12) | 26 | 63 (~6) |
| | 170-178 * | 1129 | 1.4 | TEDRWNKPQ | ......... | 916 (~81) | 213 (~19) | 33 | 70 (~6) |
| Vpr | 1-9 \hat{ } | 994 | 1.4 | MEQAPEDQG | ......... | 817 (~82) | 177 (~18) | 38 | 47 (~5) |
| | 2-10 \hat{ } | 992 | 1.5 | EQAPEDQGP | ......... | 806 (~81) | 186 (~19) | 45 | 47 (~5) |
| | 3-11 \hat{ } | 987 | 1.7 | QAPEDQGPQ | ......... | 776 (~79) | 211 (~21) | 52 | 47 (~5) |
| | 4-12 \hat{ } | 993 | 1.5 | APEDQGPQR | ......... | 823 (~83) | 170 (~17) | 50 | 39 (~4) |
| | 5-13 \hat{ }^{#} | 991 | 1.5 | PEDQGPQRE | ......... | 819 (~83) | 172 (~17) | 46 | 39 (~4) |
| | 6-14 \hat{ }^{#} | 986 | 1.5 | EDQGPQREP | ......... | 822 (~83) | 164 (~17) | 43 | 39 (~4) |
| | 18-26 \hat{ } | 1001 | 1.4 | WTLELLEEL | ......... | 809 (~81) | 192 (~19) | 39 | 93 (~9) |
| | 19-27 | 1002 | 1.4 | TLELLEELK | ......... | 809 (~81) | 193 (~19) | 39 | 94 (~9) |
| Tat | 8-16 \hat{ } | 1264 | 1.1 | LEPWKHPGS | ......... | 1090 (~86) | 174 (~14) | 30 | 32 (~3) |
| | 9-17 \hat{ } | 1264 | 1.2 | EPWKHPGSQ | ......... | 1074 (~85) | 190 (~15) | 36 | 31 (~2) |
| | 10-18 \hat{ } | 1264 | 1.0 | PWKHPGSQP | ......... | 1107 (~88) | 157 (~12) | 27 | 44 (~3) |
| | 43-51 \hat{ }^{#} | 1252 | 1.1 | LGISYGRKK | ......... | 1092 (~87) | 160 (~13) | 36 | 39 (~3) |
| | 44-52 \hat{ }^{#} | 1251 | 1.2 | GISYGRKKR | ......... | 1063 (~85) | 188 (~15) | 39 | 32 (~3) |
| | 45-53 \hat{ } | 1249 | 1.4 | ISYGRKKRR | ......... | 1037 (~83) | 212 (~17) | 43 | 29 (~2) |
| | 46-54 \hat{ } | 1245 | 1.6 | SYGRKKRRQ | ......... | 1013 (~81) | 232 (~19) | 52 | 29 (~2) |
| | 47-55 \hat{ }^{#} | 1246 | 1.2 | YGRKKRRQR | ......... | 1073 (~86) | 173 (~14) | 45 | 29 (~2) |
| | 48-56 \hat{ }^{#} | 1247 | 1.0 | GRKKRRQRR | ......... | 1104 (~89) | 143 (~11) | 44 | 40 (~3) |
| | 49-57 \hat{ } | 1245 | 1.5 | RKKRRQRRR | ......... | 1025 (~82) | 220 (~18) | 54 | 40 (~3) |
| Rev | 32-40 \hat{ } | 1396 | 1.4 | EGTRQARRN | ......... | 1156 (~83) | 240 (~17) | 40 | 69 (~5) |
| | 33-41 \hat{ } | 1396 | 0.7 | GTRQARRNR | ......... | 1295 (~93) | 101 (~7) | 29 | 24 (~2) |
| | 34-42 \hat{ } | 1396 | 0.7 | TRQARRNRR | ......... | 1285 (~92) | 111 (~8) | 30 | 24 (~2) |
| | 35-43 \hat{ } | 1396 | 0.8 | RQARRNRRR | ......... | 1277 (~91) | 119 (~9) | 28 | 24 (~2) |
| | 36-44 \hat{ } | 1396 | 0.7 | QARRNRRRR | ......... | 1285 (~92) | 111 (~8) | 27 | 24 (~2) |
| | 37-45 | 1395 | 0.6 | ARRNRRRRW | ......... | 1302 (~93) | 93 (~7) | 26 | 24 (~2) |
| | 38-46 \hat{ } | 1396 | 0.5 | RRNRRRRWR | ......... | 1323 (~95) | 73 (~5) | 23 | 20 (~1) |
| Vpu | 48-56 \hat{ }^{#$} | 1158 | 1.1 | ERAEDSGNE | ......... | 1000 (~86) | 158 (~14) | 35 | 53 (~5) |
| | 49-57 \hat{ }^{#$} | 1161 | 0.7 | RAEDSGNES | ......... | 1069 (~92) | 92 (~8) | 28 | 16 (~1) |
| Env | 33-41 | 282 | 1.1 | LWVTVYYGV | ......... | 238 (~84) | 44 (~16) | 17 | 23 (~8) |
| | (34-42) \hat{ }^{#$} | | | | | | | | |
| | 34-42 | 284 | 0.5 | WVTVYYGVP | ......... | 267 (~94) | 17 (~6) | 11 | 3 (~1) |
| | (35-43) \hat{ }^{#$} | | | | | | | | |
| | 35-43 | 284 | 0.5 | VTVYYGVPV | ......... | 269 (~95) | 15 (~5) | 11 | 2 (~1) |
| | (36-44) \hat{ }^{#$} | | | | | | | | |
| | 36-44 | 284 | 0.4 | TVYYGVPVW | ......... | 272 (~96) | 12 (~4) | 10 | 2 (~1) |
| | (37-45) \hat{ }^{#$} | | | | | | | | |
| | 37-45 | 284 | 0.5 | VYYGVPVWK | ......... | 266 (~94) | 18 (~6) | 11 | 6 (~2) |
| | (38-46) \hat{ } | | | | | | | | |
| | 38-46 | 284 | 0.8 | YYGVPVWKE | ......... | 249 (~88) | 35 (~12) | 12 | 19 (~7) |
| | (39-47) \hat{ } | | | | | | | | |
| | 39-47 | 284 | 0.9 | YGVPVWKEA | ......... | 247 (~87) | 37 (~13) | 13 | 19 (~7) |
| | (40-48) \hat{ } | | | | | | | | |
| | 40-48 | 284 | 1.0 | GVPVWKEAT | ......... | 244 (~86) | 40 (~14) | 16 | 16 (~6) |
| | (41-49) \hat{ } | | | | | | | | |
| | 41-49 | 286 | 1.0 | VPVWKEATT | ......... | 246 (~86) | 40 (~14) | 17 | 16 (~6) |
| | (42-50) \hat{ } | | | | | | | | |
| | 42-50 | 286 | 1.0 | PVWKEATTT | ......... | 247 (~86) | 39 (~14) | 17 | 16 (~6) |
| | (43-51) \hat{ } | | | | | | | | |
| | 43-51 | 286 | 1.0 | VWKEATTTL | ......... | 246 (~86) | 40 (~14) | 18 | 16 (~6) |
| | (44-52) \hat{ } | | | | | | | | |
| | 44-52 | 286 | 1.1 | WKEATTTLF | ......... | 244 (~85) | 42 (~15) | 19 | 16 (~6) |
| | (45-53) \hat{ } | | | | | | | | |
| | 45-53 | 285 | 1.1 | KEATTTLFC | ......... | 243 (~85) | 42 (~15) | 18 | 16 (~6) |
| | (46-54) \hat{ } | | | | | | | | |
| | 46-54 | 241 | 1.0 | EATTTLFCA | ......... | 205 (~85) | 36 (~15) | 13 | 17 (~7) |
| | (47-55) \hat{ } | | | | | | | | |
| | 47-55 | 241 | 0.6 | ATTTLFCAS | ......... | 222 (~92) | 19 (~8) | 11 | 6 (~2) |
| | (48-56) \hat{ } | | | | | | | | |
| | 48-56 | 241 | 0.9 | TTTLFCASD | ......... | 210 (~87) | 31 (~13) | 11 | 14 (~6) |
| | (49-57) \hat{ } | | | | | | | | |
| | 49-57 | 242 | 0.6 | TTLFCASDA | ......... | 221 (~91) | 21 (~9) | 7 | 14 (~6) |
| | (50-58) \hat{ }^{$} | | | | | | | | |
| | 50-58 | 242 | 0.7 | TLFCASDAK | ......... | 218 (~90) | 24 (~10) | 9 | 14 (~6) |
| | (51-59) \hat{ }^{#$} | | | | | | | | |
| | 65-73 | 235 | 0.9 | HNVWATHAC | ......... | 209 (~89) | 26 (~11) | 14 | 13 (~6) |
| | (66-74) \hat{ }^{#$} | | | | | | | | |
| | 66-74 | 272 | 0.7 | NVWATHACV | ......... | 248 (~91) | 24 (~9) | 12 | 13 (~5) |
| | (67-75) \hat{ }^{#$} | | | | | | | | |
| | 67-75 | 272 | 0.6 | VWATHACVP | ......... | 249 (~92) | 23 (~8) | 10 | 14 (~5) |
| | (68-76) \hat{ }^{#$} | | | | | | | | |
| | 68-76 | 287 | 0.4 | WATHACVPT | ......... | 276 (~96) | 11 (~4) | 9 | 3 (~1) |
| | (69-77) \hat{ }^{#$} | | | | | | | | |
| | 114-122 | 1032 | 1.0 | SLKPCVKLT | ......... | 884 (~86) | 148 (~14) | 21 | 61 (~6) |
| | (115-123) \hat{ }^{#} | | | | | | | | |
| | 115-123 | 1034 | 1.0 | LKPCVKLTP | ......... | 887 (~86) | 147 (~14) | 21 | 61 (~6) |
| | (116-124) \hat{ }^{#} | | | | | | | | |
| | 116-124 | 1066 | 1.1 | KPCVKLTPL | ......... | 904 (~85) | 162 (~15) | 29 | 60 (~6) |
| | (117-125) \hat{ }^{#} | | | | | | | | |
| | 117-125 | 1517 | 0.8 | PCVKLTPLC | ......... | 1357 (~89) | 160 (~11) | 29 | 59 (~4) |
| | (118-126) \hat{ }^{#} | | | | | | | | |
| | 118-126 | 1568 | 0.8 | CVKLTPLCV | ......... | 1397 (~89) | 171 (~11) | 29 | 83 (~5) |
| | (119-127) \hat{ }^{#} | | | | | | | | |
| | 119-127 | 1594 | 1.1 | VKLTPLCVS | ........T | 1374 (~86) | 220 (~14) | 33 | 83 (~5) |
| | (120-128) \hat{ }^{#} | | | | | | | | |
| | 120-128 | 2665 | 1.0 | KLTPLCVSL | .......T. | 2341 (~88) | 324 (~12) | 50 | 101 (~4) |
| | (121-129) \hat{ }^{#} | | | | | | | | |
| | 263-271 | 3685 | 1.2 | NVSTVQCTH | ......... | 3232 (~88) | 453 (~12) | 94 | 55 (~1) |
| | (241-249) \hat{ }^{#$} | | | | | | | | |
| | 264-272 | 3674 | 0.9 | VSTVQCTHG | ......... | 3382 (~92) | 292 (~8) | 70 | 39 (~1) |
| | (242-250) \hat{ }^{#} | | | | | | | | |
| | 265-273 | 3673 | 0.9 | STVQCTHGI | ......... | 3394 (~92) | 279 (~8) | 69 | 41 (~1) |
| | (243-251) \hat{ }^{#} | | | | | | | | |
| | 275-283 | 3641 | 0.7 | PVVSTQLLL | ......... | 3378 (~93) | 263 (~7) | 50 | 63 (~2) |
| | (253-261) \hat{ }^{#$} | | | | | | | | |
| | 276-284 | 3701 | 0.8 | VVSTQLLLN | ......... | 3416 (~92) | 285 (~8) | 55 | 63 (~2) |
| | (254-262) \hat{ }^{#$} | | | | | | | | |
| | 277-285 | 3833 | 0.7 | VSTQLLLNG | ......... | 3584 (~94) | 249 (~6) | 54 | 64 (~2) |
| | (255-263) \hat{ }^{#$} | | | | | | | | |
| | 278-286 | 3841 | 0.6 | STQLLLNGS | ......... | 3637 (~95) | 204 (~5) | 58 | 31 (~1) |
| | (256-264) \hat{ }^{#$} | | | | | | | | |
| | 279-287 | 3844 | 0.6 | TQLLLNGSL | ......... | 3637 (~95) | 207 (~5) | 62 | 31 (~1) |
| | (257-265) \hat{ }^{#$} | | | | | | | | |
| | 280-288 | 3857 | 0.8 | QLLLNGSLA | ......... | 3515 (~91) | 342 (~9) | 64 | 132 (~3) |
| | (258-266) \hat{ }^{#$} | | | | | | | | |
| | 281-289 | 3882 | 1.0 | LLLNGSLAE | ......... | 3495 (~90) | 387 (~10) | 65 | 136 (~4) |
| | (259-267) \hat{ }^{#} | | | | | | | | |
| | 453-461 | 3311 | 1.1 | VGKAMYAPP | ......... | 2934 (~89) | 377 (~11) | 64 | 60 (~2) |
| | (430-438) \hat{ } | | | | | | | | |
| | 454-462 | 3309 | 1.3 | GKAMYAPPI | ......... | 2840 (~86) | 469 (~14) | 77 | 65 (~2) |
| | (431-439) \hat{ } | | | | | | | | |
| | 505-513 | 527 | 0.8 | DNWRSELYK | ......... | 481 (~91) | 46 (~9) | 29 | 8 (~2) |
| | (477-485) \hat{ }^{#$} | | | | | | | | |
| | 506-514 | 529 | 0.6 | NWRSELYKY | ......... | 495 (~94) | 34 (~6) | 25 | 7 (~1) |
| | (478-486) \hat{ }^{#$} | | | | | | | | |
| | 507-515 | 533 | 0.6 | WRSELYKYK | ......... | 498 (~93) | 35 (~7) | 25 | 7 (~1) |
| | (479-487) \hat{ }^{#$} | | | | | | | | |
| | 508-516 | 537 | 0.6 | RSELYKYKV | ......... | 502 (~93) | 35 (~7) | 23 | 7 (~1) |
| | (480-488) \hat{ }^{#$} | | | | | | | | |
| | 509-517 | 541 | 0.7 | SELYKYKVV | ......... | 500 (~92) | 41 (~8) | 21 | 10 (~2) |
| | (481-489) \hat{ }^{#$} | | | | | | | | |
| | 529-537 | 557 | 1.4 | AKRRVVQRE | ......... | 455 (~82) | 102 (~18) | 31 | 40 (~7) |
| | (501-509) \hat{ } | | | | | | | | |
| | 548-556 | 1315 | 1.2 | FLGFLGAAG | ......... | 1102 (~84) | 213 (~16) | 33 | 76 (~6) |
| | (519-527) \hat{ } | | | | | | | | |
| | 549-557 | 1321 | 0.9 | LGFLGAAGS | ......... | 1143 (~87) | 178 (~13) | 23 | 89 (~7) |
| | (520-528) \hat{ } | | | | | | | | |
| | 550-558 | 1323 | 0.8 | GFLGAAGST | ......... | 1182 (~89) | 141 (~11) | 23 | 80 (~6) |
| | (521-529) \hat{ }^{#$} | | | | | | | | |
| | 551-559 | 1310 | 0.8 | FLGAAGSTM | ......... | 1168 (~89) | 142 (~11) | 23 | 80 (~6) |
| | (522-530) \hat{ }^{#$} | | | | | | | | |
| | 552-560 | 1310 | 0.7 | LGAAGSTMG | ......... | 1175 (~90) | 135 (~10) | 21 | 80 (~6) |
| | (523-531) \hat{ }^{#$} | | | | | | | | |
| | 553-561 | 1305 | 0.7 | GAAGSTMGA | ......... | 1169 (~90) | 136 (~10) | 23 | 80 (~6) |
| | (524-532) \hat{ }^{#$} | | | | | | | | |
| | 554-562 | 1307 | 0.8 | AAGSTMGAA | ......... | 1167 (~89) | 140 (~11) | 24 | 80 (~6) |
| | (525-533) ^{#$} | | | | | | | | |
| | 555-563 | 1307 | 0.6 | AGSTMGAAS | ......... | 1207 (~92) | 100 (~8) | 26 | 36 (~3) |
| | (526-534) ^{#$} | | | | | | | | |
| | 573-581 | 1614 | 1.4 | LLSGIVQQQ | ......... | 1320 (~82) | 294 (~18) | 45 | 88 (~5) |
| | (544-552) ^{#$} | | | | | | | | |
| | 595-603 | 2014 | 0.6 | LQLTVWGIK | ......... | 1877 (~93) | 137 (~7) | 31 | 36 (~2) |
| | (566-574) \hat{ }^{#} | | | | | | | | |
| | 596-604 | 2011 | 0.6 | QLTVWGIKQ | ......... | 1874 (~93) | 137 (~7) | 30 | 36 (~2) |
| | (567-575) \hat{ }^{#} | | | | | | | | |
| | 597-605 | 2039 | 0.3 | LTVWGIKQL | ......... | 1974 (~97) | 65 (~3) | 24 | 25 (~1) |
| | (568-576) \hat{ }^{#} | | | | | | | | |
| | 598-606 | 2038 | 0.4 | TVWGIKQLQ | ......... | 1961 (~96) | 77 (~4) | 26 | 25 (~1) |
| | (569-577) \hat{ }^{#$} | | | | | | | | |
| | 599-607 | 2038 | 0.4 | VWGIKQLQA | ......... | 1951 (~96) | 87 (~4) | 23 | 25 (~1) |
| | (570-578) \hat{ }^{$} | | | | | | | | |
| | 600-608 | 2042 | 0.4 | WGIKQLQAR | ......... | 1953 (~96) | 89 (~4) | 21 | 25 (~1) |
| | (571-579) \hat{ }^{$} | | | | | | | | |
| | 601-609 | 2012 | 0.9 | GIKQLQARI | ........V | 1762 (~88) | 250 (~12) | 28 | 126 (~6) |
| | (572-580) \hat{ }^{$} | | | | | | | | |
| | 602-610 | 2007 | 0.9 | IKQLQARIL | .......V. | 1765 (~88) | 242 (~12) | 28 | 126 (~6) |
| | (573-581) \hat{ }^{$} | | | | | | | | |
| | 603-611 | 1994 | 0.8 | KQLQARILA | ......V.. | 1772 (~89) | 222 (~11) | 25 | 127 (~6) |
| | (574-582) \hat{ }^{$} | | | | | | | | |
| | 618-626 | 1983 | 1.3 | DQQLLGIWG | ......... | 1671 (~84) | 312 (~16) | 40 | 82 (~4) |
| | (589-597) \hat{ } | | | | | | | | |
| | 619-627 | 1984 | 1.2 | QQLLGIWGC | ......... | 1676 (~84) | 308 (~16) | 38 | 82 (~4) |
| | (590-598) \hat{ } | | | | | | | | |
| | 620-628 | 1985 | 1.4 | QLLGIWGCS | ......... | 1611 (~81) | 374 (~19) | 41 | 83 (~4) |
| | (591-599) \hat{ } | | | | | | | | |
| | 621-629 | 1987 | 1.3 | LLGIWGCSG | ......... | 1633 (~82) | 354 (~18) | 35 | 83 (~4) |
| | (592-600) \hat{ } | | | | | | | | |
| | 622-630 | 1970 | 1.4 | LGIWGCSGK | ......... | 1581 (~80) | 389 (~20) | 38 | 96 (~5) |
| | (593-601) \hat{ } | | | | | | | | |
| | 623-631 | 1967 | 1.4 | GIWGCSGKL | ......... | 1608 (~82) | 359 (~18) | 51 | 73 (~4) |
| | (594-602) \hat{ } | | | | | | | | |
| | 624-632 | 1962 | 1.5 | IWGCSGKLI | ......... | 1599 (~81) | 363 (~19) | 54 | 73 (~4) |
| | (595-603) \hat{ } | | | | | | | | |
| | 625-633 | 1969 | 1.0 | WGCSGKLIC | ......... | 1697 (~86) | 272 (~14) | 35 | 79 (~4) |
| | (596-604) \hat{ }^{#$} | | | | | | | | |
| | 626-634 | 1966 | 1.4 | GCSGKLICT | ......... | 1599 (~81) | 367 (~19) | 43 | 78 (~4) |
| | (597-605) \hat{ }^{#$} | | | | | | | | |
| | 627-635 | 1963 | 1.4 | CSGKLICTT | ......... | 1591 (~81) | 372 (~19) | 46 | 78 (~4) |
| | (598-606) \hat{ }^{#$} | | | | | | | | |
| | 707-715 | 1141 | 1.0 | WLWYIKLFI | ......I.. | 970 (~85) | 171 (~15) | 25 | 80 (~7) |
| | (678-686) \hat{ } | | | | | | | | |
| | 848-856 | 886 | 1.1 | AIAVAEGTD | ......... | 753 (~85) | 133 (~15) | 24 | 35 (~4) |
| | (819-827) \hat{ } | | | | | | | | |
| | 849-857 | 885 | 1.2 | IAVAEGTDR | ......... | 747 (~84) | 138 (~16) | 25 | 36 (~4) |
| | (820-828) \hat{ } | | | | | | | | |
| Nef | 80-88 | 4570 | 0.8 | PQVPLRPMT | ......... | 4244 (~93) | 326 (~7) | 55 | 66 (~1) |
| | (72-80) ^{#$} | | | | | | | | |
| | 129-137 | 4557 | 1.5 | FPDWQNYTP | ......... | 3735 (~82) | 822 (~18) | 77 | 210 (~5) |
| | (121-129) ^{#$} | | | | | | | | |
| | 130-138 | 4557 | 1.5 | PDWQNYTPG | ......... | 3747 (~82) | 810 (~18) | 75 | 209 (~5) |
| | (122-130) ^{#$} | | | | | | | | |
| | 131-139 | 4555 | 1.5 | DWQNYTPGP | ......... | 3748 (~82) | 807 (~18) | 74 | 209 (~5) |
| | (123-131) ^{#$} | | | | | | | | |
| | 132-140 | 4544 | 1.5 | WQNYTPGPG | ......... | 3733 (~82) | 811 (~18) | 76 | 209 (~5) |
| | (124-132) ^{#$} | | | | | | | | |
| | 147-155 | 4623 | 1.3 | FGWCYKLVP | ....F.... | 3939 (~85) | 684 (~15) | 67 | 195 (~4) |
| | (139-147) | | | | | | | | |

^{a} Start and end alignment positions. Such positions corresponding to the HXB2 reference sequences are indicated in the brackets, only if they differ from the alignment positions. These differences are due to insertions and deletions in the protein alignment.
^{b} The total number of HIV-1 clade B protein sequences obtained at the respective nonamer positions of the protein sequence alignment. The number of sequences for each nonamer position varies due to the inclusion of both partial and full-length sequences.
^{c} Shannon's nonamer entropy.
^{d} The nonamer sequence corresponding to the HXB2 reference sequence. Insertions to the alignment with respect to the HXB2 sequence are shown as gaps “-”.
^{e} The primary nonamer is the peptide with the highest incidence at a given nonamer position in the protein alignment. Residues that are identical to the HXB2 sequence is denoted as “.” whereas residues that are different have their amino acids displayed. For example, at position 1-9 of Gag, the HXB2 sequence have identical sequence to that of the primary nonamer thus the primary nonamer have the sequence “.........” displayed. However at position, 22-30 in Gag, the last residue in the nonamer differs from that of HXB2, having R instead of K, and thus the nonamer sequence is shown as “........R”.
^{f} Total variants of the primary nonamers are all sequences that differ by one or more amino acids from the primary nonamer at the corresponding position in the protein alignment.
^{g} The number of unique variants at the indicated nonamer position.
^{h} The primary variant is the most common (highest incidence) variant nonamer at the indicated nonamer position of the protein alignment.
* Highly conserved nonamers that is HIV-1 specific, i.e. the nonamers is not matched to any other reported protein in the NCBI protein database (as of November 2010).
\hat{ } Highly conserved nonamers that is primate lentivirus group specific (taxonomy id 11652), i.e. the nonamers matched to any reported proteins of the primate lentivirus group from the NCBI protein database (as of November 2010).
^{#} Highly conserved clade B nonamers that are also highly conserved in clade C with a primary nonamer incidence of 80% or more, the primary variant incidence of less than 10% and 100 or more nonamers analysed at that position.
^{$} Highly conserved clade B nonamers that are also highly conserved in clades A and C with a primary nonamer incidence of 80% or more, the primary variant incidence of less than 10% and 100 or more nonamers analysed at that position.
⁺ An example interpretation of the table: The primary nonamer MGARASVLS was present in 945 sequences (~82%) of all 1156 sequences analyzed at nonamer position 1-9 in the Gag protein alignment. The remaining 211 sequences (~18%) at that position were variants of the primary nonamer and comprised 33 unique peptides, one of which is the primary variant and is present in about 10% (110) of all the 1156 analysed sequences. The remaining 101 variants at that position were represented by 36 additional variant sequences.

### Example 7

### HIV-1 and/or Primate Lentivirus Group Specific Highly Conserved Nonamers, with Possible Multiclade Conservation

BLAST search of the 504 highly conserved nonamers of clade B against all reported sequences of nature revealed that two were specific to HIV-1 with no matching 9 consecutive amino acid identity, while 374 were primate lentivirus group specific, with several showing multiclade conservation (Table 6). For example, of the 504 HIV-1 clade B conserved nonamers, 330 were biclade conserved and 84 were triclade conserved (Table 6). When contiguous nonamers were joined, there were 64 biclade and 24 triclade highly conserved sequences (Table 7).

TABLE 7
Highly conserved HIV-1 clade B and C sequences.

| Protein | Positions ^{a} | Sequences ^{b} |
|---|---|---|
| Gag | 36-45 | WASRELERFA |
| | 135-143 | SQNYPIVQN |
| | (129-137) | |
| | 154-164 | SPRTLNAWVKV |
| | (148-158) | |
| | 168-178 | KAFSPEVIPMF |
| | (162-172) | |
| | 180-208 | ALSEGATPQDLNTMLNTVGGHQAAM |
| | (174-202) | QMLK |
| | 210-220 | TINEEAAEWDR |
| | (204-214) | |
| | 235-247 | REPRGSDIAGTTS |
| | (229-241) | |
| | 275-285 | GLNKIVRMYSP |
| | (269-279) | |
| | 293-306 | QGPKEPFRDYVDRF |
| | (287-300) | |
| | 326-340 | TLLVQNANPDCKTIL |
| | (320-324) | |
| | 349-362 | LEEMMTACQGVGGP |
| | (343-356) | |
| | 364-374 | HKARVLAEAMS |
| | (358-368) | |
| | 398-407 | KCFNCGKEGH |
| | (391-400) | |
| | 439-447 | NFLGKIWPS |
| | (432-440) | |
| | 449-457 | KGRPGNFLQ |
| | (442-450) | |
| Pol | 57-65 | PQITLWQRP |
| | 77-90 | EALLDTGADDTVLE |
| | 100-109 | PKMIGGIGGF |
| | 103-112 | IGGIGGFIKV |
| | 150-174 | GCTLNFPISPIETVPVKLKPGMDGP |
| | 176-189 | VKQWPLTEEKIKAL |
| | 204-214 | KIGPENPYNTP |
| | 226-237 | WRKLVDFRELNK |
| | 239-257 | TQDFWEVQLGIPHPAGLKK |
| | 259-272 | KSVTVLDVGDAYFS |
| | 279-289 | FRKYTAFTIPS |
| | 291-316 | NNETPGIRYQYNVLPQGWKGSPAIFQ |
| | 340-350 | DDLYVGSDLEI |
| | 375-399 | KHQKEPPFLWMGYELHPDKWTVQPI |
| | 406-426 | SWTVNDIQKLVGKLNWASQIY |
| | 453-471 | EAELELAENREILKEPVHG |
| | 716-724 | FLDGIDKAQ |
| | 755-764 | CDKCQLKGEA |
| | 766-786 | HGQVDCSPGIWQLDCTHLEGK |
| | 788-798 | ILVAVHVASGY |
| | 800-814 | EAEVIPAETGQETAY |
| | 817-826 | LKLAGRWPVK |
| | 841-850 | VKAACWWAGI |
| | 852-870 | QEFGIPYNPQSQGVVESMN |
| | 872-881 | ELKKIIGQVR |
| | 883-902 | QAEHLKTAVQMAVFIHNFKR |
| | 904-915 | GGIGGYSAGERI |
| | 934-944 | KIQNFRVYYRD |
| | 938-946 | FRVYYRDSR |
| | 950-970 | WKGPAKLLWKGEGAVVIQDNS |
| Vif | 52-60 | SSEVHIPLG |
| | 138-150 | GHNKVGSLQYLAL |
| Vpr | 5-14 | PEDQGPQREP |
| Tat | 43-52 | LGISYGRKKR |
| | 47-56 | YGRKKRRQRR |
| Vpu | 48-57 | ERAEDSGNES |
| Env | 33-44 | LWVTVYYGVPVW |
| | (34-45) | |
| | 50-58 | TLFCASDAK |
| | (51-59) | |
| | 65-76 | HNVWATHACVPT |
| | (66-77) | |
| | 114-128 | SLKPCVKLTPLCVTL |
| | (115-129) | |
| | 263-273 | NVSTVQCTHGI |
| | (241-251) | |
| | 275-289 | PVVSTQLLLNGSLAE |
| | (253-267) | |
| | 505-517 | DNWRSELYKYKVV |
| | (477-489) | |
| | 550-563 | GFLGAAGSTMGAAS |
| | (521-534) | |
| | 573-581 | LLSGIVQQQ |
| | (544-552) | |
| | 595-606 | LQLTVWGIKQLQ |
| | (566-577) | |
| | 625-635 | WGCSGKLICTT |
| | (596-606) | |
| Nef | 80-88 | PQVPLRPMT |
| | (72-80) | |
| | 129-140 | FPDWQNYTPGPG |
| | (121-132) | |

^{a} Start and end alignment positions. Such positions corresponding to the HXB2 reference sequences are indicated in the brackets, only if they differ from the alignment positions. These differences are due to insertions and deletions in the protein alignment.
^{b} Sequences of 9 or more amino acids formed by one or by joining more than two contiguous nonamers that have primary clade B nonamer percentage incidence(s) of more than 80% and less than 10% representation of the primary variant in the Glade B and C protein alignments, respectively. Sequences with less than 100 nonamers in at that given nonamer position will be ignored. SEQ ID NOs for each peptide are identified in Table 5 and corresponding nonamers in Table 6.

### Example 8

### Close Correspondence of Clade B Conserved Sequences of Reported Epitopes

A search of the HIV Molecular Immunology Database revealed that of the 78 highly conserved HIV-1 clade B sequences, 39 matched at least nine consecutive amino acids of reported human T-cell epitopes (Table 8). These epitopes were restricted by 68 HLAs of class I alleles and 34 class II, with several promiscuous to multiple HLA alleles (HLA-supertype restricted). Twenty-one of the 39 matched conserved sequences contained the full epitope sequences. Additionally, seven of the highly conserved clade B sequences shared at least nine amino acids of Elispot positive peptides HLA-DR4 transgenic mice (Table 8) (Simon et al., 2010).

TABLE 8
Correlation of reported human T-cell epitopes and HLA-DR4 transgenic
mouse epitope peptides to highly conserved HIV-1 clade B and bi-clade sequences.
SEQ ID NOs: 1141-1272, in the order as shown for the reported T-cell epitope peptides.

| | | | Reported Epitopes | | Record |
|---|---|---|---|---|---|
| Protein | Position ^{a} | Highly conserved sequences ^{b} | Sequences ^{c} | HLA | number/Ref |
| Gag | 16-25 | WEKIRLRPGG | EKIRLRPGGKKKYKL_{(B)} | DQB1*0301, | 201064 |
| | | | | DQB1*0601, | |
| | | | | DRB1*1303, | |
| | | | | DRB1*1502, | |
| | | | | DRB3*0101, | |
| | | | | DRB5*0102 | |
| | 35-45 | VWASRELERFA | YKLKHIVWASRELER_{(B)} | DQB1*0301, | 201065 |
| | | | | DQB1*0601, | |
| | | | | DRB1*1303, | |
| | | | | DRB1*1502, | |
| | | | | DRB3*0101, | |
| | | | | DRB5*0102 | |
| | | | HIVWASRELERFAVN_{(B)} | DQB1*0301, | 201066 |
| | | | | DQB1*0601, | |
| | | | | DRB1*1303, | |
| | | | | DRB1*1502, | |
| | | | | DRB3*0101, | |
| | | | | DRB5*0102 | |
| | | | LVWASRELERF₍_{C)} | A*3002, | 53056, |
| | | | | B*5703 | 53958 |
| | | | WASRELERF_{(B)} | B*3501 | 55, 1200 |
| | | | ASRELERFAVNPGLL_{(B)} | DRB*0101, | 201008, |
| | | | | DRB*0401, | (Simon et |
| | | | | DRB1*0401, | al., 2010) |
| | | | | DRB1*0405, | |
| | | | | DRB1*0701, | |
| | | | | DRB1*1302, | |
| | | | | DRB1*1501 | |
| | 135-143 | SQNYPIVQN | SQNYPIVQNIQ_{(B)} | A*2402 | 53592 |
| | (129-137) | | | | |
| | 154-164 | SPRTLNAWVKV | SPRTLNAWV_{(B)} | B*8101 | 146,148 |
| | (148-158) | | | | |
| | | | ISPRTLNAWV_{(C)} | B*5702, | 56469 |
| | | | | B*5703 | |
| | 166-178 | EEKAFSPEVIPMF | EEKAFSPEV_{(A,B,C,D)} | B*4501, | 53305, |
| | (160-172) | | | B*4415, | 200518, |
| | | | | DRB1*0101, | 52248, |
| | | | | DRB1*1501, | 55862 |
| | | | | DRB1*0101 | |
| | | | TLNAWVKVVEEKAFSPEVIP_{(B)} | DRB1*0405, | 201112 |
| | | | | DRB1*0701, | |
| | | | | DRB1*1302, | |
| | | | | DRB1*1503, | |
| | | | | DRB1*0701, | |
| | | | | DRB1*1601 | |
| | | | EKAFSPEVIPMFSALSEGAT_{(B)} | DRB1*0701, | 201124 |
| | | | | DRB1*1601 | |
| | | | EKAFSPEVIPMFSAL_{(B)} | DRB*0401 | (Simon et |
| | | | | | al., 2010) |
| | | | KAFSPEVIPMF_{(B,C)} | A*3402, | 162, 163, |
| | | | | A*7401, | 165, 1976, |
| | | | | A*0201, | 52082, |
| | | | | B*0801, | 52086, |
| | | | | B*5701, | 52191, |
| | | | | B*5703, | 1976, |
| | | | | B*5801, | 52082, |
| | | | | Cw*0302, | 52086, |
| | | | | Cw*0701 | 52191, |
| | | | | | 52622, |
| | | | | | 52760, |
| | | | | | 52844, |
| | | | | | 55001, |
| | | | | | 55092, |
| | | | | | 55766, |
| | | | | | 55670, |
| | | | | | 53635, |
| | | | | | 54625, |
| | | | | | 56751, |
| | | | | | 53921 |
| | | | IEEKAFSPEVI_{(C)} | B*4501 | 53964 |
| | | | FSPEVIPMF_{(B,C)} | A*8001 | 53665 |
| | 180-208 | ALSEGATPQDLNTMLNTVG | SALSEGATPQDLNMMLNIVG_{(A)} | B*8101 | 178 |
| | (174-202) | GHQAAMQMLK | | | |
| | | | MFSALSEGATPQDLNTMLNT_{(B)} | DRB1*1302, | 201113 |
| | | | | DRB1*1503 | |
| | | | IPMFSALSEGATPQD_{(B)} | DRB*0401 | (Simon et |
| | | | | | al., 2010) |
| | | | PQDLNTMLNTVGGHQ_{(B)} | DRB1*1302, | 201114 |
| | | | | DRB1*1503 | |
| | | | LSEGATPQDL_{(B)} | A*2902, | 53232 |
| | | | | B*0801, | |
| | | | | B*4403 | |
| | | | ATPQDLNTMLNT_{(C)} | B*5802 | 55681 |
| | | | TPQDLNTML_{(A,B,C,D)} | A*3001, | 186, 1149, |
| | | | | A*3303, | 1156, |
| | | | | A*3402, | 1977, |
| | | | | A*7401, | 56243, |
| | | | | B*0702, | 53058, |
| | | | | B*3910, | 53300, |
| | | | | B*4201, | 53922, |
| | | | | B*5301, | 53923, |
| | | | | B*8101, | 53966, |
| | | | | Cw*0401, | 53967, |
| | | | | Cw*0802 | 54593, |
| | | | | | 54610, |
| | | | | | 54611, |
| | | | | | 54612, |
| | | | | | 54613, |
| | | | | | 54622, |
| | | | | | 55674, |
| | | | | | 56576 |
| | | | PQDLNTMLN_{(B)} | A*6801 | 55557 |
| | | | DLNTMLNIV_{(B)} | B*1402 | 196 |
| | | | GHQAAMQML_{(B,C)} | B*3901, | 201, |
| | | | | B*1510 | 53334, |
| | | | | | 53968 |
| | 210-220 | TINEEAAEWDR | ETINEEAAEWDRVHPVHA_{(B)} | DRB1*0101, | 200511 |
| | (204-214) | | | DRB1*1501, | |
| | | | | DRB1*0101 | |
| | | | LKETINEEAAEWDRVHPVHA_{(B)} | DRB1*1302, | 201119 |
| | | | | DRB1*1503, | |
| | | | | DRB1*0405, | |
| | | | | DRB1*0701 | |
| | | | LKDTINEEAAEWDRLHPV_{(C)} | A*6801 | 53969 |
| | | | DTINEEAAEW_{(B)} | B*5301 | 2005 |
| | | | ETINEEAAEW_{(B)} | A*0201, | 2006, |
| | | | | A*2501, | 54147, |
| | | | | A*3002, | 54921 |
| | | | | B*0702, | |
| | | | | B*1801, | |
| | | | | B*5101, | |
| | | | | B*5301, | |
| | | | | Cw*0102, | |
| | | | | Cw*1203 | |
| | | | ETINEEAAEWDRVHPVHAGPIA_{(B)} | DRB1*0101, | 200521 |
| | | | | DRB1*1501, | |
| | | | | DRB1*0101 | |
| | | | INEEAAEWDRV_{(B)} | DRB1*0101 | 201058 |
| | 231-253 | PGQMREPRGSDIAGTTSTL | GSDIAGTTSTQEQI_{(B)} | DQB1*0301, | 201068 |
| | (225-247) | QEQI | | DQB1*0601, | |
| | | | | DRB1*1303, | |
| | | | | DRB1*1502, | |
| | | | | DRB3*0101, | |
| | | | | DRB5*0102 | |
| | | | GSDIAGTTSTLQEQI_{(B)} | DRB*0401 | (Simon et |
| | | | | | al., 2010) |
| | | | PRGSDIAGTTSTLQEQIGWM_{(B)} | DRB1*1302, | 201116 |
| | | | | DRB1*1503, | |
| | | | | DRB1*0405, | |
| | | | | DRB1*0701, | |
| | | | | DRB1*0301, | |
| | | | | DRB1*0401 | |
| | | | GPIAPGQMREPRGSDIAGTT_{(B)} | DRB1*0301, | 201121 |
| | | | | DRB1*0401 | |
| | | | GQMREPRGSDI_{(B,C)} | A*0301, | 54585 |
| | | | | A*3001, | |
| | | | | B*1301, | |
| | | | | B*1402, | |
| | | | | Cw*0602, | |
| | | | | Cw*0802 | |
| | | | HAGPIAPGQMREPRG_{(B)} | B*3501, | 55285 |
| | | | | A*0201 | |
| | 259-269 | NPPIPVGEIYK | TNNPPIPVGEIYKRWIILGL_{(B)} | DRB1*0503, | 201122 |
| | (253-263) | | | DRB1*1302 | |
| | | | PPIPVGEIY_{(B)} | B7 Supertype | 52963 |
| | 275-285 | GLNKIVRMYSP | GLNKIVRMYSPTSIL_{(B)} | DRB*0401 | (Simon et |
| | (269-279) | | | | al., 2010) |
| | | | WIILGLNKIVRMYSPTSI_{(B)} | DRB1*0101, | 201036 |
| | | | | DRB1*0401, | |
| | | | | DRB1*0405, | |
| | | | | DRB1*0701, | |
| | | | | DRB1*1101, | |
| | | | | DRB1*1302, | |
| | | | | DRB1*1501, | |
| | | | | DRB5*0101 | |
| | | | ILGLNKIVRMY_{(B)} | DRB1*0401, | 201043, |
| | | | | DRB1*1302, | 52952 |
| | | | | DRB1*1501, | |
| | | | | B7 Supertype | |
| | | | GLNKIVRMYSPTSIL_{(B)} | DQB1*0301, | 201070 |
| | | | | DQB1*0601, | |
| | | | | DRB1*1303, | |
| | | | | DRB1*1502, | |
| | | | | DRB3*0101, | |
| | | | | DRB5*0102 | |
| | | | WIILGLNKIVRMYSP_{(B)} | DQB1*0602, | 201076 |
| | | | | DQB1*0604, | |
| | | | | DRB1*1302, | |
| | | | | DRB1*1501, | |
| | | | | DRB3*0301, | |
| | | | | DRB5*0101, | |
| | | | | DQB1*0301, | |
| | | | | DQB1*0601, | |
| | | | | DRB1*1303, | |
| | | | | DRB1*1502, | |
| | | | | DRB3*0101, | |
| | | | | DRB5*0102 | |
| | | | IYKRWIILGLNKIVRMYSPT_{(B)} | DRB1*0503, | 201123 |
| | | | | DRB1*1302 | |
| | | | NKIVRMYSPTSILDIRQGPK_{(B)} | DRB1*0701, | 201125 |
| | | | | DRB1*1601 | |
| | | | KRWIILGLNKIVRMYSPTSI_{(B)} | DRB1*0101, | 201134 |
| | | | | DRB1*0301, | |
| | | | | DRB1*0401, | |
| | | | | DRB1*0405, | |
| | | | | DRB1*0701, | |
| | | | | DRB1*1101, | |
| | | | | DRB1*1302 | |
| | | | GLNKIVRMY_{(B)} | A*1103, | 302, 53245 |
| | | | | B*1501, | |
| | | | | A*2402, | |
| | | | | B*1402, | |
| | | | | B*1501, | |
| | | | | Cw*0802 | |
| | | | NKIVRMYSPVSILDI_{(A,AG)} | DPA*0201, | 55892 |
| | | | | DPB1*0101, | |
| | | | | DPB1*1301, | |
| | | | | DRB1*1301 | |
| | 293-315 | QGPKEPFRDYVDRFYKTLR | SILDIKQGPKEPFRD_{(A,B)} | A*0308, | 55898, |
| | (287-309) | AEQA | | DPA*0103, | (Simon et |
| | | | | DRB*0401 | al., 2010) |
| | | | RQGPKEPFRDYVDRF_{(A,AG,B)} | DPA*0201, | 55895 |
| | | | | DPB1*0101, | |
| | | | | DPB1*1301, | |
| | | | | DRB1*1301 | |
| | | | PKEPFRDYV_{(B)} | DRB1*0101, | 200515 |
| | | | | DRB1*1501 | |
| | | | EPFRDYVDRFYKTLRAEQAS_{(B)} | DRB1*1302, | 201117, |
| | | | | DRB1*1503, | 201118 |
| | | | | DRB1*0405, | |
| | | | | DRB1*0701, | |
| | | | | DRB1*1601, | |
| | | | | DRB1*0301, | |
| | | | | DRB1*0401 | |
| | | | EPFRDYVDRF_{(B,D)} | A*0201 | 54148, |
| | | | | | 55882 |
| | | | FRDYVDRFYK_{(B,D)} | B*1801, | 309, 310 |
| | | | | A*0201, | |
| | | | | A*2501, | |
| | | | | B*1801, | |
| | | | | B*5101, | |
| | | | | Cw*0102, | |
| | | | | Cw*1203 | |
| | | | FRDYVDRFYKTLRAE_{(A,D)} | A*0101, | 53615 |
| | | | | A*7401, | |
| | | | | B*5801 | |
| | | | RDYVDRFYKTL_{(B)} | B*4402 | 315 |
| | | | DYVDRFYKTLR_{(B)} | A*3303 | 52723 |
| | | | DYVDRFYKT_{(B)} | A*1103, | 53246 |
| | | | | A*2402, | |
| | | | | B*1402, | |
| | | | | B*1501, | |
| | | | | Cw*0802 | |
| | | | YVDRFYKTLRAEQASQEV_{(B)} | DRB1*0101, | 201007 |
| | | | | DRB1*0401, | |
| | | | | DRB1*0405, | |
| | | | | DRB1*0701, | |
| | | | | DRB1*1101, | |
| | | | | DRB1*1302, | |
| | | | | DRB1*1501, | |
| | | | | DRB5*0101 | |
| | | | YVDRFYKTL_{(B)} | A*0207 | 52330 |
| | | | VDRFYKTLRAEQASQ_{(B)} | DQB1*0602, | 201077 |
| | | | | DQB1*0604, | |
| | | | | DRB1*1302, | |
| | | | | DRB1*1501, | |
| | | | | DRB3*0301, | |
| | | | | DRB5*0101, | |
| | | | | DQB1*0301, | |
| | | | | DQB1*0601, | |
| | | | | DRB1*1303, | |
| | | | | DRB1*1502, | |
| | | | | DRB3*0101, | |
| | | | | DRB5*0102, | |
| | | | | DQB1*0301, | |
| | | | | DRB1*0401, | |
| | | | | DRB1*1101, | |
| | | | | DRB3*0202, | |
| | | | | DRB4*0103, | |
| | | | | DQB1*0202, | |
| | | | | DQB1*0602, | |
| | | | | DRB1*0701, | |
| | | | | DRB1*1501, | |
| | | | | DRB4*0103, | |
| | | | | DRB5*0101 | |
| | | | DRFYKTLRA_{(B)} | B*1402, | 324, 328, |
| | | | | Cw*0702, | 52623 |
| | | | | Cw*0802 | |
| | | | DRFYKTLRAEQ_{(B)} | A*2902, | 53222 |
| | | | | B*1402, | |
| | | | | Cw*0802 | |
| | | | RFYKTLRAEQAS_{(B)} | DRB1*0101, | 201042 |
| | | | | DRB1*0401, | |
| | | | | DRB1*0405, | |
| | | | | DRB1*0701, | |
| | | | | DRB1*1101, | |
| | | | | DRB1*1501, | |
| | | | | DRB5*0101 | |
| | | | FYKTLRAEQASQE_{(B)} | DRB1*0101, | 201044 |
| | | | | DRB1*0401, | |
| | | | | DRB1*0405, | |
| | | | | DRB1*1101, | |
| | | | | DRB1*1501, | |
| | | | | DRB5*0101 | |
| | | | FYKTLRAEQASQ_{(B)} | DRB1*0101, | 201045 |
| | | | | DRB1*0401, | |
| | | | | DRB1*1101, | |
| | | | | DRB5*0101 | |
| | | | YKTLRAEQA_{(B)} | DRB1*0101 | 201046 |
| | | | YKTLRAEQASQ_{(B)} | DRB1*1302 | 201060 |
| | 319-345 | VKNWMTETLLVQNANPDCK | DCKTILKAL_{(B)} | B*0801 | 353 |
| | (313-339) | TILKALGP | | | |
| | | | MTDTLLVQNANPDCKTIL_{(C)} | B*0801 | 53979 |
| | | | VKNWMTETLLVQNAN_{(B)} | DRB*0401 | (Simon et |
| | | | | | al., 2010) |
| | | | VKNWMTETLL_{(B)} | A*6801 | 55568 |
| | | | NANPDCKTILRAL_{(C)} | B*3910 | 56477 |
| | 398-407 | KCFNCGKEGH | GNFRNQRKIVKCFNCGKEGH_{(B)} | DRB1*0101, | 200513 |
| | (391-400) | | | DRB1*1501, | |
| | | | | DRB1*1501 | |
| | 439-447 | NFLGKIWPS | RQANFLGKIWPSHKGR_{(B)} | DR01*0401, | 201011 |
| | (432-440) | | | DRB1*0101, | |
| | | | | DRB1*0405, | |
| | | | | DRB1*1101, | |
| | | | | DRB1*1302, | |
| | | | | DRB1*1501, | |
| | | | | DRB5*0101 | |
| Pol | 100-109 | PKMIGGIGGF | KMIGGIGGFI_{(B)} | A*0201 | 53441 |
| | 150-174 | GCTLNFPISPIETVPVKLK | FPISPIETVP_{(B)} | A*0206, | 55524 |
| | | PGMDGP | | B*4801, | |
| | | | | B*5401 | |
| | | | FPISPIETV_{(B)} | B*5401 | 55527 |
| | | | PISPIETVPVKLKPGM₍_{C)} | B*3910 | 53990 |
| | | | SPIETVPVKL _{©} | B*8101 | 53931, |
| | | | | | 54623, |
| | | | | | 56487 |
| | 176-189 | VKQWPLTEEKIKAL | GMDGPKVKQWPLTEEKIK_{(C)} | B*4202 | 53991 |
| | 239-257 | TQDFWEVQLGIPHPAGLKK | FWEVQLGIPHPAGLKKKK_{(C)} | A*6801 | 53993 |
| | 259-272 | KSVTVLDVGDAYFS | TVLDVGDAY_{(B)} | A*0201, | 53604 |
| | | | | A*0301, | |
| | | | | B*3501 | |
| | | | KKKSVTVLDVGDAYFSV_{(C)} | Cw*0401 | 53994 |
| | 291-316 | NNETPGIRYQYNVLPQGWK | NNETPGIRY_{(C)} | B*1801 | 56489 |
| | | GSPAIFQ | | | |
| | | | LPQGWKGSPAI_{(C)} | B*3910 | 53999 |
| | | | LPQGWKGSPA_{(B)} | B*5401 | 55528 |
| | 375_399 | KHQKEPPFLWMGYELHPDK | DKKHQKEPPFLWMGYELH_{(C)} | B*1510 | 54004 |
| | | WTVQP I | | | |
| | 401-426 | LPEKDSWTVNDIQKLVGKL | TVQPIQLPEKDSWTVNDI_{(C)} | B*5301 | 54005 |
| | | NWASQIY | | | |
| | | | EKDSWTVNDIQKLVGKL_{(C)} | A*0205 | 54006 |
| | | | KLVGKLNWA_{(A,B,C,D)} | A2 Supertype | 56508 |
| | | | KLNWASQIY_{(B,C)} | A*3002 | 53329, |
| | | | | | 54007 |
| | 453-471 | EAELELAENREILKEPVHG | ELAENREILKEPVHGVYY_{(C)} | Cw*0202 | 54009 |
| | 750-758 | EIVASCDKC | PPIVAKEIVASCDKCQLK_{(C)} | B*8101 | 54029 |
| | | | EIVASCDKCQL_{(C)} | B*4201 | 56501 |
| | 788-815 | ILVAVHVASGYIEAEVIPA | GKIILVAVHVASGYI_{(B)} | DRB1*0101, | 201138 |
| | | ETGQETAYF | | DRB1*0401, | |
| | | | | DRB1*0405, | |
| | | | | DRB1*0701, | |
| | | | | DRB1*1101, | |
| | | | | DRB1*1302, | |
| | | | | DRB1*1501, | |
| | | | | DRB5*0101 | |
| | | | PAETGQETAYFILKLAGR_{(C)} | A*6802 | 54031 |
| | | | HVASGYIEA_{(B)} | B*5401 | 55529 |
| | | | AETGQETAYY_{(C)} | B*4403 | 56503 |
| | 817-826 | LKLAGRWPVK | ILKLAGRWPVK_{(C)} | A*0301 | 53941 |
| | 844-870 | ACWWAGIKQEFGIPYNPQS | IQQEFGIPYNPQ_{(C)} | B*1503 | 56505 |
| | | QGVVESMN | | | |
| | | | IPYNPQSQGVV_{(A,B,C,D)} | B7 Supertype | 56525 |
| | 876-915 | IIGQVRDQAEHLKTAVQMA | QVRDQAEHL_{(C)} | A*0205 | 54034 |
| | | VFIHNFKRKGGIGGYSAGE | | | |
| | | RI | | | |
| | | | QMAVFIHNFK_{(A,B,C,D)} | A3 Supertype | 56513 |
| | | | AVFIHNFKRK_{(B,CRF01}_AE) | A*1101 | 1192, |
| | | | | | 52101 |
| | | | FKRKGGIGGY_{(B,C)} | B*1503 | 53291, |
| | | | | | 53943, |
| | | | | | 54184, |
| | | | | | 56506, |
| | | | | | 55076 |
| | | | RKGGIGGYSAGERIVDII_{(B)} | A*0101, | 201257 |
| | | | | A*0201, | |
| | | | | B*4001, | |
| | | | | Cw*0304, | |
| | | | | DRB1*0801, | |
| | | | | DRB1*1301 | |
| | 934-944 | KIQNFRVYYRD | KIQNFRVYY_{(B,C)} | A*2501, | 1966, |
| | | | | A*3001, | 53284, |
| | | | | A*0205, | 54605, |
| | | | | A*3002, | 54927, |
| | | | | B*0702, | 54954 |
| | | | | B*1402, | |
| | | | | B*1801, | |
| | | | | B*4201, | |
| | | | | B*4202, | |
| | | | | B*4403, | |
| | | | | B*4426, | |
| | | | | B*4430, | |
| | | | | B*5301, | |
| | | | | Cw*0401, | |
| | | | | Cw*0802 | |
| | | | TKIQNFRVYY_{(B,C)} | B*1503 | 55073 |
| | | | KIQNFRVYYR_{(A,B,C,D)} | A*3303, A3 | 52721, |
| | | | | Supertype | 56514 |
| | 948-970 | PLWKGPAKLLWKGEGAVVI | LWKGEGAVVIQDNSDIKV_{(B)} | A*0101, | 201259 |
| | | QDNS | | A*0201, | |
| | | | | B*4001, | |
| | | | | Cw*0304, | |
| | | | | DRB1*0801, | |
| | | | | DRB1*1301 | |
| Vpr | 18-27 | WTLELLEELK | GPQREPYNEWTLELLEEL_{(C)} | Cw*0704 | 54044 |
| Tat | 43-57 | LGISYGRKKRRQRRR | KALGISYGRKKRRQR_{(B)} | DRB1*0404, | 201266 |
| | | | | DRB1*0701, | |
| | | | | DRB1*0101, | |
| | | | | DRB1*1302, | |
| | | | | DRB1*1101, | |
| | | | | DRB1*1104, | |
| | | | | DRB1*0301, | |
| | | | | DRB1*1501, | |
| | | | | DRB1*1301, | |
| | | | | DRB1*1501, | |
| | | | | DRB1*0701, | |
| | | | | DRB1*0901, | |
| | | | | DRB1*1101, | |
| | | | | DRB1*1301, | |
| | | | | DRB1*1301, | |
| | | | | DRB1*1501 | |
| Vpu | 48-57 | ERAEDSGNES | ERAEDSGNESEGDTEELSA_{(C)} | A*2301, | 56262 |
| | | | | A*2902, | |
| | | | | B*4101, | |
| | | | | B*4201, | |
| | | | | Cw*1701 | |
| Env | 33-58 | LWVTVYYGVPVWKEATTTL | KLWVTVYYGV_{(B)} | A*0201, | 628, 55290 |
| | (34-59) | FCASDAK | | B*3501 | |
| | | | LWVTVYYGV_{(B)} | A*0201 | 52703 |
| | | | LWVTVYYGVPVWKEATTTLFCA_{(B)} | B*3501, | 55287 |
| | | | | A*0201 | |
| | | | TVYYGVPVWK_{(A,B,C,D)} | A*0301 | 631, 633, |
| | | | | | 634, 1119, |
| | | | | | 1120, |
| | | | | | 52432, |
| | | | | | 55150 |
| | | | TVYYGVPVW_{(A,B,C,D)} | A*2902, | 53812 |
| | | | | A*2902, | |
| | | | | B*1503, | |
| | | | | B*1801, | |
| | | | | Cw*0202, | |
| | | | | Cw*1203, | |
| | | | | A*3001, | |
| | | | | A*6601, | |
| | | | | B*5703, | |
| | | | | B*5801, | |
| | | | | Cw*0401, | |
| | | | | Cw*1801 | |
| | | | TVYYGVPVWKEAKTTLF_{(C)} | A*4301, | 54053, |
| | | | | A*3201, | 54055, |
| | | | | B*5801 | 54056 |
| | | | VTVYYGVPVWK_{(A,B,C,D)} | A3 Supertype | 56515 |
| | | | TTLFCASDAK_{(A,B,C,D)} | A3 Supertype | 56519 |
| | 114-128 | SLKPCVKLTPLCVTL | KLTPLCVTL_{(A,B,C,D)} | A*0201, A2 | 671, 56509 |
| | (115-129) | | | Supertype | |
| | 707-715 | WLWYIKIFI | WLWYIKIFI(B) | A*0201, | 854, 55295 |
| | (678-686) | | | B*3501 | |
| Nef | 80-88 | PQVPLRPMT | TPQVPLRPMTY(B) | B7 Supertype | 52960 |
| | (72-80) | | | | |
| | | | FPVRPQVPLRPMTYK(B) | B*1503 | 54442 |
| | | | PVRPQVPLRPMTYKA(B) | A*0201, | 55317 |
| | | | | B*3501 | |
| | 129-140 | FPDWQNYTPGPG | FPDWQNYTP(B) | B*5401 | 55530 |
| | (121-132) | | | | |
| | 147-155 | FGWCFKLVP | GIRYPLTFGWCFKLVP(B) | A*6801 | 55537 |
| | (139-147) | | | | |

^{a} Start and end positions. Cross reference to the alignments positions are made with the HXB2 reference sequences and the HXB2 positions might be different from the reference HXB2 sequences due to insertions. and deletions in the protein alignments. HXB2 sequence positions differing from the protein alignment positions are shown within brackets.
^{b} Highly conserved clade B sequences. SEQ ID NOs for each peptide are identified in Table 5 and for the corresponding nonamers in Table 6.
^{c} Epitope sequences matching nine or more amino acids of the highly conserved HIV-1 clade B sequence are underlined. The clades that the epitopes are restricted to are shown in the brackets.

### REFERENCES

The disclosure of each reference cited is expressly incorporated herein.
Abram, M. E., Ferris, A. L., Shao, W., Alvord, W. G. and Hughes, S. H. (2010). Nature, position, and frequency of mutations made in a single cycle of HIV-1 replication, J Virol, 84, 9864-78.
Allen, T. M., Altfeld, M., Geer, S. C., Kalife, E. T., Moore, C., O'Sullivan K, M., Desouza, I., Feeney, M. E., Eldridge, R. L., Maier, E. L., Kaufmann, D. E., Lahaie, M. P., Reyor, L., Tanzi, G., Johnston, M. N., Brander, C., Draenert, R., Rockstroh, J. K., Jessen, H., Rosenberg, E. S., Mallal, S. A. and Walker, B. D. (2005). Selective escape from CD8+ T-cell responses represents a major driving force of human immunodeficiency virus type 1 (HIV-1) sequence diversity and reveals constraints on HIV-1 evolution, J Virol, 79, 13239-49.
Allen, T. M., Altfeld, M., Yu, X. G., O'Sullivan, K. M., Lichterfeld, M., Le Gall, S., John, M., Mothe, B. R., Lee, P. K., Kalife, E. T., Cohen, D. E., Freedberg, K. A., Strick, D. A., Johnston, M. N., Sette, A., Rosenberg, E. S., Mallal, S. A., Goulder, P. J., Brander, C. and Walker, B. D. (2004). Selection, transmission, and reversion of an antigen-processing cytotoxic T-lymphocyte escape mutation in human immunodeficiency virus type 1 infection, J Virol, 78, 7069-78.
Altschul, S. F., Gish, W., Miller, W., Myers, E. W. and Lipman, D. J. (1990). Basic local alignment search tool, J Mol Biol, 215, 403-10.
Brumme, Z. L., John, M., Carlson, J. M., Brumme, C. J., Chan, D., Brockman, M. A., Swenson, L. C., Tao, I., Szeto, S., Rosato, P., Sela, J., Kadie, C. M., Frahm, N., Brander, C., Haas, D. W., Riddler, S. A., Haubrich, R., Walker, B. D., Harrigan, P. R., Heckerman, D. and Mallal, S. (2009). HLA-associated immune escape pathways in HIV-1 subtype B Gag, Pol and Nef proteins, PLoS One, 4, e6687.
Brumme, Z. L. and Walker, B. D. (2009). Tracking the culprit: HIV-1 evolution and immune selection revealed by single-genome amplification, J Exp Med, 206, 1215-8.
Draenert, R., Le Gall, S., Pfafferott, K. J., Leslie, A. J., Chetty, P., Brander, C., Holmes, E. C., Chang, S. C., Feeney, M. E., Addo, M. M., Ruiz, L., Ramduth, D., Jeena, P., Altfeld, M., Thomas, S., Tang, Y., Verrill, C. L., Dixon, C., Prado, J. G., Kiepiela, P., Martinez-Picado, J., Walker, B. D. and Goulder, P. J. (2004). Immune selection for altered antigen processing leads to cytotoxic T lymphocyte escape in chronic HIV-1 infection, J Exp Med, 199, 905-15.
Eigen, M. (1993). Viral quasispecies, Sci Am, 269, 42-9.
Gotch, F. (1998). Cross-clade T cell recognition of HIV.1, Curr Opin Immunol, 10, 388-92.
Goulder, P. J. and Watkins, D. I. (2004). HIV and SIV CTL escape: implications for vaccine design, Nat Rev Immunol, 4, 630-40.
Jetzt, A. E., Yu, H., Klarmann, G. J., Ron, Y., Preston, B. D. and Dougherty, J. P. (2000). High rate of recombination throughout the human immunodeficiency virus type 1 genome, J Virol, 74, 1234-40.
Jones, R. B., Yue, F. Y., Gu, X. X., Hunter, D. V., Mujib, S., Gyenes, G., Mason, R. D., Mohamed, R., MacDonald, K. S., Kovacs, C. and Ostrowski, M. A. (2009). Human immunodeficiency virus type 1 escapes from interleukin-2-producing CD4+ T-cell responses without high-frequency fixation of mutations, J Virol, 83, 8722-32.
Kelleher, A. D., Long, C., Holmes, E. C., Allen, R. L., Wilson, J., Conlon, C., Workman, C., Shaunak, S., Olson, K., Goulder, P., Brander, C., Ogg, G., Sullivan, J. S., Dyer, W., Jones, I., McMichael, A. J., Rowland-Jones, S. and Phillips, R. E. (2001). Clustered mutations in HIV-1 gag are consistently required for escape from HLA-B27-restricted cytotoxic T lymphocyte responses, J Exp Med, 193, 375-86.
Khan, A. M., Miotto, O., Nascimento, E. J., Srinivasan, K. N., Heiny, A. T., Zhang, G. L., Marques, E. T., Tan, T. W., Brusic, V., Salmon, J. and August, J. T. (2008). Conservation and variability of dengue virus proteins: implications for vaccine design, PLoS Negl Trop Dis, 2, e272.
Korber, B. T., Letvin, N. L. and Haynes, B. F. (2009). T-cell vaccine strategies for human immunodeficiency virus, the virus with a thousand faces, J Virol, 83, 8300-14.
Leslie, A. J., Pfafferott, K. J., Chetty, P., Draenert, R., Addo, M. M., Feeney, M., Tang, Y., Holmes, E. C., Allen, T., Prado, J. G., Altfeld, M., Brander, C., Dixon, C., Ramduth, D., Jeena, P., Thomas, S. A., St John, A., Roach, T. A., Kupfer, B., Luzzi, G., Edwards, A., Taylor, G., Lyall, H., Tudor-Williams, G., Novelli, V., Martinez-Picado, J., Kiepiela, P., Walker, B. D. and Goulder, P. J. (2004). HIV evolution: CTL escape mutation and reversion after transmission, Nat Med, 10, 282-9.
Letourneau, S., Im, E. J., Mashishi, T., Brereton, C., Bridgeman, A., Yang, H., Dorrell, L., Dong, T., Korber, B., McMichael, A. J. and Hanke, T. (2007). Design and pre-clinical evaluation of a universal HIV-1 vaccine, PLoS One, 2, e984.
Liang, B., Luo, M., Ball, T. B., Yao, X., Van Domselaar, G., Cuff, W. R., Cheang, M., Jones, S. J. and Plummer, F. A. (2008). Systematic analysis of host immunological pressure on the envelope gene of human immunodeficiency virus type 1 by an immunobioinformatics approach, Curr HIV Res, 6, 370-9.
McMichael, A. J., Borrow, P., Tomaras, G. D., Goonetilleke, N. and Haynes, B. F. (2010). The immune response during acute HIV-1 infection: clues for vaccine development, Nat Rev Immunol, 10, 11-23.
Miotto, 0., Heiny, A., Tan, T. W., August, J. T. and Brusic, V. (2008). Identification of human-to-human transmissibility factors in PB2 proteins of influenza A by large-scale mutual information analysis, BMC Bioinformatics, 9 Suppl 1, S18.
Okazaki, T., Pendleton, C. D., Lemonnier, F. and Berzofsky, J. A. (2003). Epitope-enhanced conserved HIV-1 peptide protects HLA-A2-transgenic mice against virus expressing HIV-1 antigen, J Immunol, 171, 2548-55.
Pei, J., Kim, B. H. and Grishin, N. V. (2008). PROMALS3D: a tool for multiple protein sequence and structure alignments, Nucleic Acids Res, 36, 2295-300.
Perelson, A. S., Neumann, A. U., Markowitz, M., Leonard, J. M. and Ho, D. D. (1996). HIV-1 dynamics in vivo: virion clearance rate, infected cell life-span, and viral generation time, Science, 271, 1582-6.
Pereyra, F., Jia, X., McLaren, P. J., Telenti, A., de Bakker, P. I., Walker, B. D., Ripke, S., Brumme, C. J., Pulit, S. L., Carrington, M., Kadie, C. M., Carlson, J. M., Heckerman, D., Graham, R. R., Plenge, R. M., Deeks, S. G., Gianniny, L., Crawford, G., Sullivan, J., Gonzalez, E., Davies, L., Camargo, A., Moore, J. M., Beattie, N., Gupta, S., Crenshaw, A., Burtt, N. P., Guiducci, C., Gupta, N., Gao, X., Qi, Y., Yuki, Y., Piechocka-Trocha, A., Cutrell, E., Rosenberg, R., Moss, K. L., Lemay, P., O'Leary, J., Schaefer, T., Verma, P., Toth, I., Block, B., Baker, B., Rothchild, A., Lian, J., Proudfoot, J., Alvino, D. M., Vine, S., Addo, M. M., Allen, T. M., Altfeld, M., Henn, M. R., Le Gall, S., Streeck, H., Haas, D. W., Kuritzkes, D. R., Robbins, G. K., Shafer, R. W., Gulick, R. M., Shikuma, C. M., Haubrich, R., Riddler, S., Sax, P. E., Daar, E. S., Ribaudo, H. J., Agan, B., Agarwal, S., Ahern, R. L., Allen, B. L., Altidor, S., Altschuler, E. L., Ambardar, S., Anastos, K., Anderson, B., Anderson, V., Andrady, U., Antoniskis, D., Bangsberg, D., Barbaro, D., Barrie, W., Bartczak, J., Barton, S., Basden, P., Basgoz, N., Bazner, S., Bellos, N. C., Benson, A. M., Berger, J., Bernard, N. F., Bernard, A. M., Birch, C., Bodner, S. J., Bolan, R. K., Boudreaux, E. T., Bradley, M., Braun, J. F., Brndjar, J. E., Brown, S. J., Brown, K., Brown, S. T., Burack, J., Bush, L. M., Cafaro, V., Campbell, O., Campbell, J., Carlson, R. H., Carmichael, J. K., Casey, K. K., Cavacuiti, C., Celestin, G., Chambers, S. T., Chez, N., Chirch, L. M., Cimoch, P. J., Cohen, D., Cohn, L. E., Conway, B., Cooper, D. A., Cornelson, B., Cox, D. T., Cristofano, M. V., Cuchural, G., Jr., Czartoski, J. L., Dahman, J. M., Daly, J. S., Davis, B. T., Davis, K., Davod, S. M., DeJesus, E., Dietz, C. A., Dunham, E., Dunn, M. E., Ellerin, T. B., Eron, J. J., Fangman, J. J., Farel, C. E., Ferlazzo, H., Fidler, S., Fleenor-Ford, A., Frankel, R., Freedberg, K. A., French, N. K., Fuchs, J. D., Fuller, J. D., Gaberman, J., Gallant, J. E., Gandhi, R. T., Garcia, E., Garmon, D., Gathe, J. C., Jr., Gaultier, C. R., Gebre, W., Gilman, F. D., Gilson, I., Goepfert, P. A., Gottlieb, M. S., Goulston, C., Groger, R. K., Gurley, T. D., Haber, S., Hardwicke, R., Hardy, W. D., Harrigan, P. R., Hawkins, T. N., Heath, S., Hecht, F. M., Henry, W. K., Hladek, M., Hoffman, R. P., Horton, J. M., Hsu, R. K., Huhn, G. D., Hunt, P., Hupert, M. J., Illeman, M. L., Jaeger, H., Jellinger, R. M., John, M., Johnson, J. A., Johnson, K. L., Johnson, H., Johnson, K., Joly, J., Jordan, W. C., Kauffman, C. A., Khanlou, H., Killian, R. K., Kim, A. Y., Kim, D. D., Kinder, C. A., Kirchner, J. T., Kogelman, L., Kojic, E. M., Korthuis, P. T., Kurisu, W., Kwon, D. S., LaMar, M., Lampiris, H., Lanzafame, M., Lederman, M. M., Lee, D. M., Lee, J. M., Lee, M. J., Lee, E. T., Lemoine, J., Levy, J. A., Llibre, J. M., Liguori, M. A., Little, S. J., Liu, A. Y., Lopez, A. J., Loutfy, M. R., Loy, D., Mohammed, D. Y., Man, A., Mansour, M. K., Marconi, V. C., Markowitz, M., Marques, R., Martin, J. N., Martin, H. L., Jr., Mayer, K. H., McElrath, M. J., McGhee, T. A., McGovern, B. H., McGowan, K., McIntyre, D., McLeod, G. X., Menezes, P., Mesa, G., Metroka, C. E., Meyer-Olson, D., Miller, A. O., Montgomery, K., Mounzer, K. C., Nagami, E. H., Nagin, I., Nahass, R. G., Nelson, M. O., Nielsen, C., Norene, D. L., O'Connor, D. H., Ojikutu, B. O., Okulicz, J., Oladehin, O. O., Oldfield, E. C., 3rd, Olender, S. A., Ostrowski, M., Owen, W. F., Jr., Pae, E., Parsonnet, J., Pavlatos, A. M., Perlmutter, A. M., Pierce, M. N., Pincus, J. M., Pisani, L., Price, L. J., Proia, L., Prokesch, R. C., Pujet, H. C., Ramgopal, M., Rathod, A., Rausch, M., Ravishankar, J., Rhame, F. S., Richards, C. S., Richman, D. D., Rodes, B., Rodriguez, M., Rose, R. C., 3rd, Rosenberg, E. S., Rosenthal, D., Ross, P. E., Rubin, D. S., Rumbaugh, E., Saenz, L., Salvaggio, M. R., Sanchez, W. C., Sanjana, V. M., Santiago, S., Schmidt, W., Schuitemaker, H., Sestak, P. M., Shalit, P., Shay, W., Shirvani, V. N., Silebi, V. I., Sizemore, J. M., Jr., Skolnik, P. R., Sokol-Anderson, M., Sosman, J. M., Stabile, P., Stapleton, J. T., Starrett, S., Stein, F., Stellbrink, H. J., Sterman, F. L., Stone, V. E., Stone, D. R., Tambussi, G., Taplitz, R. A., Tedaldi, E. M., Theisen, W., Torres, R., Tosiello, L., Tremblay, C., Tribble, M. A., Trinh, P. D., Tsao, A., Ueda, P., Vaccaro, A., Valadas, E., Vanig, T. J., Vecino, I., Vega, V. M., Veikley, W., Wade, B. H., Walworth, C., Wanidworanun, C., Ward, D. J., Warner, D. A., Weber, R. D., Webster, D., Weis, S., Wheeler, D. A., White, D. J., Wilkins, E., Winston, A., Wlodaver, C. G., van't Wout, A., Wright, D. P., Yang, O. O., Yurdin, D. L., Zabukovic, B. W., Zachary, K. C., Zeeman, B. and Zhao, M. (2010). The major genetic determinants of HIV-1 control affect HLA class I peptide presentation, Science, 330, 1551-7.
R_Development_Core_Team (2008) R: a language and environment for statistical computing. R Foundation for Statistical Computing, Vienna, Austria.
Rammensee, H. G. (1995). Chemistry of peptides associated with MHC class I and class II molecules, Curr Opin Immunol, 7, 85-96.
Rychert, J., Saindon, S., Placek, S., Daskalakis, D. and Rosenberg, E. (2007). Sequence variation occurs in CD4 epitopes during early HIV infection, J Acquir Immune Defic Syndr, 46, 261-7.
Salazar-Gonzalez, J. F., Salazar, M. G., Keele, B. F., Learn, G. H., Giorgi, E. E., Li, H., Decker, J. M., Wang, S., Baalwa, J., Kraus, M. H., Parrish, N. F., Shaw, K. S., Guffey, M. B., Bar, K. J., Davis, K. L., Ochsenbauer-Jambor, C., Kappes, J. C., Saag, M. S., Cohen, M. S., Mulenga, J., Derdeyn, C. A., Allen, S., Hunter, E., Markowitz, M., Hraber, P., Perelson, A. S., Bhattacharya, T., Haynes, B. F., Korber, B. T., Hahn, B. H. and Shaw, G. M. (2009). Genetic identity, biological phenotype, and evolutionary pathways of transmitted/founder viruses in acute and early HIV-1 infection, J Exp Med, 206, 1273-89.
Shannon, C. E. (1948). A mathematical theory of communication, Bell System Technical Journal, 27, 379-423 and 623-656.
Simon, G. G., Hu, Y., Khan, A. M., Zhou, J., Salmon, J., Chikhlikar, P. R., Jung, K. O., Marques, E. T. and August, J. T. (2010). Dendritic cell mediated delivery of plasmid DNA encoding LAMP/HIV-1 Gag fusion immunogen enhances T cell epitope responses in HLA DR4 transgenic mice, PLoS ONE, 5, e8574.
Sloan-Lancaster, J. and Allen, P. M. (1996). Altered peptide ligand-induced partial T cell activation: molecular mechanisms and role in T cell biology, Annu Rev Immunol, 14, 1-27.
Thompson, J. D., Higgins, D. G. and Gibson, T. J. (1994). CLUSTAL W: improving the sensitivity of progressive multiple sequence alignment through sequence weighting, position-specific gap penalties and weight matrix choice, Nucleic Acids Res, 22, 4673-80.
Thompson, J. D., Thierry, J. C. and Poch, O. (2003). RASCAL: rapid scanning and correction of multiple sequence alignments, Bioinformatics, 19, 1155-61.
Troyer, R. M., McNevin, J., Liu, Y., Zhang, S. C., Krizan, R. W., Abraha, A., Tebit, D. M., Zhao, H., Avila, S., Lobritz, M. A., McElrath, M. J., Le Gall, S., Mullins, J. I. and Arts, E. J. (2009). Variable fitness impact of HIV-1 escape mutations to cytotoxic T lymphocyte (CTL) response, PLoS Pathog, 5, e1000365.
Wang, Y. E., Li, B., Carlson, J. M., Streeck, H., Gladden, A. D., Goodman, R., Schneidewind, A., Power, K. A., Toth, I., Frahm, N., Alter, G., Brander, C., Carrington, M., Walker, B. D., Altfeld, M., Heckerman, D. and Allen, T. M. (2009). Protective HLA class I alleles that restrict acute-phase CD8+ T-cell responses are associated with viral escape mutations located in highly conserved regions of human immunodeficiency virus type 1, J Virol, 83, 1845-55.
Wickham, H. (2009) ggplot2: elegant graphics for data analysis. Springer New York.
Wilson, C. C., McKinney, D., Anders, M., MaWhinney, S., Forster, J., Crimi, C., Southwood, S., Sette, A., Chesnut, R., Newman, M. J. and Livingston, B. D. (2003). Development of a DNA vaccine designed to induce cytotoxic T lymphocyte responses to multiple conserved epitopes in HIV-1, J Immunol, 171, 5611-23.
Yang, O. O. (2009). Candidate vaccine sequences to represent intra- and inter-clade HIV-1 variation, PLoS One, 4, e7388.
Yokomaku, Y., Miura, H., Tomiyama, H., Kawana-Tachikawa, A., Takiguchi, M., Kojima, A., Nagai, Y., Iwamoto, A., Matsuda, Z. and Ariyoshi, K. (2004). Impaired processing and presentation of cytotoxic-T-lymphocyte (CTL) epitopes are major escape mechanisms from CTL immune pressure in human immunodeficiency virus type 1 infection, J Virol, 78, 1324-32.

We claim:

1. A polypeptide comprising: one or more discontinuous segments of HIV-1 clade B proteins, said segments comprising from 9 to 40 contiguous amino acid residues, wherein said segments comprise at least one nonamer, wherein each nonamer is represented in the NCBI Entrez protein database of HIV-1 clade B proteins as of August 2008 at a frequency of greater than 80% and for which the maximum representation of individual variants from the amino acid sequence of said segments is less than 10% in said database.

2. The polypeptide of claim 1 comprising a segment of HIV-1 selected from the group consisting of: SEQ ID NO: 637-1140.

3. The polypeptide of claim 1 comprising a segment of HIV-1 selected from the group consisting of SEQ ID NO: 55-132.

4. The polypeptide of claim 1 which further comprises: (a) a LAMP-1 lumenal sequence comprising SEQ ID NO: 1273; and (b) a LAMP transmembrane and cytoplasmic tail comprising SEQ ID NO: 1274, wherein the lumenal sequence is amino-terminal to the one or more discontinuous segments which are amino-terminal to the LAMP transmembrane and cytoplasmic tail.

5. The polypeptide of claim 1 wherein the maximum representation of individual variants from the amino acid sequence of said segments is less than 5% in said database.

6. The polypeptide of claim 1 wherein the polypeptide comprises not more than one of said segments.

7. The polypeptide of claim 1 wherein the polypeptide comprises a plurality of said segments.

8. A polynucleotide encoding the polypeptide of claim 1 or 4.

9. The polynucleotide of claim 8 wherein codons encoding the polypeptide are optimized according to most frequent human codon usage.

10. The polynucleotide of claim 8 comprising SEQ ID NO: 1275 encoding the LAMP-1 lumenal sequence and SEQ ID NO: 1276 encoding the transmembrane and cytoplasmic tail of LAMP-1.

11. A nucleic acid vector which comprises the polynucleotide of claim 8.

12. The nucleic acid vector of claim 11 which is a DNA virus.

13. The nucleic acid vector of claim 11 which is a RNA virus.

14. The nucleic acid vector of claim 11 which is a plasmid.

15. A host cell which comprises a nucleic acid vector of claim 11.

16. The host cell of claim 15 which is an antigen presenting cell.

17. The host cell of claim 15 which is a dendritic cell.

18. A method of producing a polypeptide comprising, culturing a host cell according to claim 15 under conditions in which the host cell expresses the polypeptide.

19. The method of claim 18 further comprising, harvesting the peptide from the culture medium or host cells.

20. A method of producing a cellular vaccine comprising:
transfecting antigen presenting cells with a nucleic acid vector according to claim 11, whereby the antigen presenting cells express the polypeptide.

21. The method of claim 20 wherein the antigen presenting cells are dendritic cells.

22. A method of making a vaccine, comprising: mixing together the polypeptide of claim 1 and an immune adjuvant.

23. The method of claim 22 wherein the adjuvant is selected from the group consisting of alum, lecithin, squalene, Toll-like receptor (TLR) adaptor molecules, and combinations thereof.

24. A vaccine composition comprising the polypeptide of claim 1 or 4.

25. A method of immunizing a human or other animal subject, comprising:
administering to the human or other animal subject a polypeptide of claim 1 or a nucleic acid vector according to claim 11 or a host cell according to claim 15, in an amount effective to elicit HIV-specific T-cell activation.

26. The method of claim 25 further comprising administering to the subject a boost comprising the polypeptide of claim 1.

27. The method of claim 25 further comprising administering an immune adjuvant to the subject.

28. The method of claim 25 wherein the administration is oral, mucosal, nasal, intramuscular, intravenous, intradermal, intranasal, subcutaneous, or via electroporation.

29. A method of identifying species of a primate lentivirus, comprising:
hybridizing a polynucleotide according to claim 8 or its complement to genomic nucleic acid of the primate lentivirus or its complement, wherein hybridization of the genome or its complement to the polynucleotide or its complement identifies the lentivirus as HIV-1, as of clade B, as of biclade B and C, as of triclade A, B, and C, or as of pan-clade A, B, C and D.

30. The method of claim 29 wherein the polynucleotide is from 15-120 nucleotides in length.

31. A method of identifying a primate lentivirus, comprising:
contacting an antibody which specifically binds to a polypeptide of claim 1 to proteins from a cell infected by the primate lentivirus, wherein specific binding of the antibody to the proteins indicates presence of the primate lentivirus.

32. A method of identifying a primate lentivirus in a patient, comprising:
contacting a polypeptide of claim 1 with a blood sample from the patient, wherein specific binding of the polypeptide to an antibody in the blood sample or to T cells in the blood sample indicates presence of the primate lentivirus.
